(19) United States
(12) Patent Application Publication
(10) Pub. No.: US 2026/0209228 A1
(43) Pub. Date: Jul. 23, 2026

(54) BICYCLIC DERIVATIVES

(71) Applicant: Elanco Tiergesundheit AG, Basel (CH)

(72) Inventors: Chouaib TAHTAOUI, Rixheim (FR); Denise RAGEOT, Saint-Louis (FR); Francois PAUTRAT, Mulhouse (FR); Pierre DUCRAY, Village-Neuf (FR)

(21) Appl. No.: 19/331,388

(22) Filed: Sep. 17, 2025

Related U.S. Application Data
(63) Continuation of application No. 17/415,323, filed on Dec. 13, 2019, now abandoned.
(60) Provisional application No. 62/781,073, filed on Dec. 18, 2018.

(51) Int. Cl. C07D 471/04; A61P 33/10; C07D 311/68; C07D 405/12; C07D 493/04; C07D 495/04

(52) U.S. Cl. CPC C07D 471/04; A61P 33/10; C07D 311/68; C07D 405/12; C07D 493/04; C07D 495/04

(57) ABSTRACT

The present invention provides compounds of formula (I):

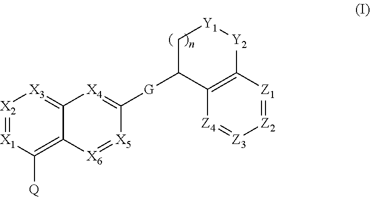

which are useful in the control of endoparasites, for example heartworms, in warm-blooded animals.

### CROSS-REFERENCE TO RELATED APPLICATIONS

This application is a Continuation application of U.S. application Ser. No. 17/415,323, filed Jun. 17, 2021, which is a National Stage entry of International Application No. PCT/US2019/066298, filed Dec. 13, 2019, which claims priority to U.S. Patent Application No. 62/781,073, filed Dec. 18, 2018.

### FIELD

The present invention relates to medicinal chemistry, pharmacology, and veterinary and human medicine. More particularly, the present invention relates to compounds of formula (I) and their use in the control of endoparasites, for example heartworms, in warm-blooded animals.

### BACKGROUND

Heartworm (Dirofilaria immitis) is a parasitic roundworm that is spread from host to host through the bites of mosquitoes. The lifecycle starts when a female mosquito takes a blood meal from an infected host. The mosquito ingests immature heartworms which then molt to the infective larvae stage and travel to the mosquitoes' mouth parts. The mosquito then feeds on a susceptible host, such as a dog or cat, depositing the infective larvae. The larvae then molt to the next larval stage in the new host and then migrate through the body, eventually ending up in the blood vessels. As the larvae migrate through the tissues they molt into juvenile adults. The juvenile adults eventually move into the blood vessels of the lungs where they mature into sexually active adults. The adult heartworms then breed and release immature heartworms completing the cycle. Heartworm infection may result in serious disease for the host.

Adult heartworm infections may be treated with arsenic-based compounds; the treatment is time consuming, cumbersome, and often only partly successful. Accordingly, treatment is focused on the control of heartworm infection. Heartworm control is currently performed exclusively by year round periodical administration of drugs. Typical treatments include macrocyclic lactones such as ivermectin, moxidectin, and milbemycin oxime. Unfortunately, developing resistance of Dirofilaria immitis to macrocyclic lactones has been observed. Accordingly, there is a need for new compounds which effectively control heartworm infections either by way of prophylaxis or by directly killing heartworms. Certain treatments of endoparasites are described in WO 2017/178416, WO 2018/087036, WO 2018/197401, WO 2019/025341, and WO 2019/002132.

### SUMMARY

The present invention provides compounds of formula (I) which effectively treat and/or control endoparasites (e.g., heartworm) in warm-blooded animals.

In one embodiment, the present invention provides compounds of formula (I):

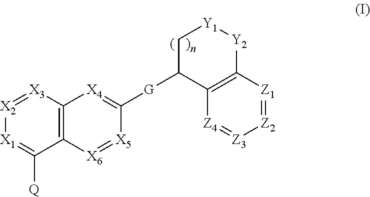

wherein
n is 0 or 1;
X₁ is selected from the group consisting of N and CR₁;
X₂ is selected from the group consisting of N and CR₂;
X₃ is selected from the group consisting of N and CR₃;
X₄ is selected from the group consisting of N and CR₄;
X₅ is selected from the group consisting of N and CR₅;
X₆ is selected from the group consisting of N and CR₆;
   wherein at least one of X₁, X₂, X₃, and X₅ is N or wherein none of X₁, X₂, X₃, X₄, X₅, and X₆ are N;
G is the group

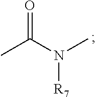

Y₁ is selected from the group consisting of CR₈R₉, O, S, and NR₁₀;
Y₂ is selected from the group consisting of CR₈R₉, O, S, and NR₁₀;
   wherein at least one of the groups Y₁ or Y₂ is CR₈R₉;
Z₁ is selected from the group consisting of N, O, S, and CR₁₁;
Z₂ is selected from the group consisting of nil, N, and CR₁₁;
Z₃ is selected from the group consisting of nil, N and CR₁₁;
Z₄ is selected from the group consisting of N, O, S, and CR₁₁;
   wherein no more than 2 of Z₁, Z₂, Z₃, and Z₄ are N and wherein only one of Z₁ and
   Z₄ is O or S, Z₂ is nil only when Z₁ is O or S, and Z₃ is nil only when Z₄ is O or S;
R₁ is selected from the group consisting of hydrogen, halogen, hydroxyl, —SH, —SC₁-C₄ alkyl, —S(O)(C₁-C₄ alkyl, —S(O)₂(C₁-C₄ alkyl, cyano, C₁-C₄ alkyl, C₁-C₄ halogenoalkyl, C₁-C₄-alkoxy, —B(OR₁₂)(OR₁₃) wherein R₁₂ is, each time taken, selected from the group consisting or hydrogen, C₁-C₄ alkyl, and C₃-C₆ cycloalkyl, R₁₃ is, each time taken, selected from the group consisting or hydrogen, C₁-C₄ alkyl, and C₃-C₆ cycloalkyl, or R₁₂ and R₁₃ together with the oxygen atoms to which they are attached form a 5- to 7-membered ring which is optionally substituted with 1 to 4 C₁-C₄ alkyl; —NH₂, —NH(C₁-C₄ alkyl), and —N(C₁-C₄ alkyl)₂;
R₂ is selected from the group consisting of hydrogen, halogen, hydroxyl, —SH, —SC₁-C₄ alkyl, —S(O)(C₁-C₄ alkyl, —S(O)₂(C₁-C₄ alkyl, cyano, C₁-C₄ alkyl, C₁-C₄ halogenoalkyl, C₁-C₄-alkoxy, —B(OR₁₂)(OR₁₃) wherein R₁₂ is, each time taken, selected from the group consisting or hydrogen, C₁-C₄ alkyl, and C₃-C₆ cycloalkyl, R₁₃ is, each time taken, selected from the group consisting or hydrogen, C₁-C₄ alkyl, and C₃-C₆ cycloalkyl, or R₁₂ and R₁₃ together with the oxygen atoms to which they are attached form a 5- to 7-membered ring which is optionally substituted with 1 to 4 C₁-C₄ alkyl; —NH₂, —NH(C₁-C₄ alkyl), and —N(C₁-C₄ alkyl)₂;
R₃ is selected from the group consisting of hydrogen, halogen, hydroxyl, —SH, —SC₁-C₄ alkyl, —S(O)(C₁-C₄ alkyl, —S(O)₂(C₁-C₄ alkyl, cyano, C₁-C₄ alkyl, C₁-C₄ halogenoalkyl, C₁-C₄-alkoxy, —B(OR₁₂)(OR₁₃) wherein R₁₂ is, each time taken, selected from the group consisting or hydrogen, C₁-C₄ alkyl, and C₃-C₆ cycloalkyl, R₁₃ is, each time taken, selected from the group consisting or hydrogen, C₁-C₄ alkyl, and C₃-C₆ cycloalkyl, or R₁₂ and R₁₃ together with the oxygen atoms to which they are attached form a 5- to 7-membered ring which is optionally substituted with 1 to 4 C₁-C₄ alkyl; —NH₂, —NH(C₁-C₄ alkyl), and —N(C₁-C₄ alkyl)₂;
R₄ is selected from the group consisting of halogen, cyano, —CHO, hydroxyl, C₁-C₄ alkyl, C₂-C₄ alkenyl, C₂-C₄ alkynyl, C₃-C₆ cycloalkyl, C₁-C₄ halogenoalkyl, C₁-C₄-alkoxy substituted-C₁-C₄ alkyl, benzyl optionally substituted with 1 to 5 halogen atoms, C₁-C₄ alkoxy, —NH₂, —NH(C₁-C₄ alkyl), —N(C₁-C₄ alkyl)₂, —NH(C₃-C₆ cycloalkyl), —N(C₁-C₄ alkyl)(C₃-C₆-cycloalkyl), —NH(4- to 7-membered heterocycloalkyl), —N(C₁-C₄ alkyl)(4- to 7-membered heterocycloalkyl), —N(C₁-C₄ alkyl)(C₃-C₆-cycloalkyl), —N(C₁-C₄ alkyl)(C₁-C₄ alkoxy), —C(O)NH(C₁-C₄ alkyl), —C(O)N(C₁-C₄ alkyl)₂, —NHSO₂(C₁-C₄ alkyl), —SC₁-C₄ alkyl, —S(O)C₁-C₄ alkyl, —SO₂C₁-C₄ alkyl, —B(OR₁₂)(OR₁₃) wherein R₁₂ is, each time taken, selected from the group consisting or hydrogen, C₁-C₄ alkyl, and C₃-C₆ cycloalkyl, R₁₃ is, each time taken, selected from the group consisting or hydrogen, C₁-C₄ alkyl, and C₃-C₆ cycloalkyl, or R₁₂ and R₁₃ together with the oxygen atoms to which they are attached form a 5- to 7-membered ring which is optionally substituted with 1 to 4 C₁-C₄ alkyl; 6- or 10 membered aryl; and a monocyclic heterocycle selected from the group of 4- to 7-membered heterocycloalkyl, 5-membered heteroaryl having at least one nitrogen atom via which the 5-membered heteroaryl ring is connected to the rest of the molecule; 6-membered heteroaryl having at least one nitrogen atom; each of the aryl, heterocycloalkyl, and heteroaryl ring in R₄ is optionally substituted with 1, 2 or 3 substituents independently selected from the group consisting of halogen, cyano, nitro, hydroxyl, oxo, C₁-C₄ alkyl, C₃-C₆ cycloalkyl, C₁-C₄ halogenoalkyl, C₁-C₄ alkoxy, —NH₂, —NH(C₁-C₄ alkyl), —N(C₁-C₄ alkyl)₂, —NH(C₃-C₆ cycloalkyl), —N(C₁-C₄ alkyl)(C₃-C₆-cycloalkyl), —NHSO₂(C₁-C₄ alkyl), —SC₁-C₄ alkyl, —S(O)C₁-C₄ alkyl, —SO₂C₁-C₄ alkyl, —S(O)C₁-C₄-halogenoalkyl and —SO₂C₁-C₄ halogenoalkyl; and wherein each C₁-C₄ alkyl, C₃-C₆ cycloalkyl and C₁-C₄ alkoxy in R₄ may be optionally substituted with 1, 2 or 3 substituents independently selected from the group consisting of halogen, hydroxyl, —NH₂, —NH(C₁-C₄ alkyl), —N(C₁-C₄ alkyl)₂, cyano, carboxyl, carbamoyl, C₁-C₄ alkoxycarbonyl, —C(O)NH(C₁-C₄ alkyl), —C(O)N(C₁-C₄ alkyl)₂, and C₁-C₄ alkoxy;
R₅ is selected from the group consisting of hydrogen, halogen, hydroxyl, —SH, —SC₁-C₄ alkyl, —S(O)(C₁-C₄ alkyl, —S(O)₂(C₁-C₄ alkyl, cyano, C₁-C₄ alkyl, C₁-C₄ halogenoalkyl, C₁-C₄-alkoxy, —B(OR₁₂)(OR₁₃) wherein R₁₂ is, each time taken, selected from the group consisting or hydrogen, C₁-C₄ alkyl, and C₃-C₆ cycloalkyl, R₁₃ is, each time taken, selected from the group consisting or hydrogen, C₁-C₄ alkyl, and C₃-C₆ cycloalkyl, or R₁₂ and R₁₃ together with the oxygen atoms to which they are attached form a 5- to 7-membered ring which is optionally substituted with 1 to 4 C₁-C₄ alkyl; —NH₂, —NH(C₁-C₄ alkyl), and —N(C₁-C₄ alkyl)₂;
R₆ is selected from the group consisting of hydrogen, halogen, hydroxyl, —SH, —SC₁-C₄ alkyl, —S(O)(C₁-C₄ alkyl, —S(O)₂(C₁-C₄ alkyl, cyano, C₁-C₄ alkyl, C₁-C₄ halogenoalkyl, C₁-C₄-alkoxy, —B(OR₁₂)(OR₁₃) wherein R₁₂ is, each time taken, selected from the group consisting or hydrogen, C₁-C₄ alkyl, and C₃-C₆ cycloalkyl, R₁₃ is, each time taken, selected from the group consisting or hydrogen, C₁-C₄ alkyl, and C₃-C₆ cycloalkyl, or R₁₂ and R₁₃ together with the oxygen atoms to which they are attached form a 5- to 7-membered ring which is optionally substituted with 1 to 4 C₁-C₄ alkyl; —NH₂, —NH(C₁-C₄ alkyl), and —N(C₁-C₄ alkyl)₂;
R₇ is selected from the group consisting of hydrogen, C₁-C₄ alkyl, and C₃-C₆ cycloalkyl optionally substituted with 1 to 5 halogen atoms, —C(H)O, C₂-C₄ alkenyl, C₂-C₄ alkynyl, C₁-C₄ halogenoalkyl, and C₁-C₄-alkoxy;
R₈ is, each time selected, independently selected from the group consisting of hydrogen, fluoro, and C₁-C₄ alkyl;
R₉ is, each time selected, independently selected from the group consisting of hydrogen, fluoro, and C₁-C₄ alkyl;
R₁₀ is selected from the group consisting of hydrogen and C₁-C₄ alkyl;
R₁₁ is, each time selected, independently selected from the group consisting of hydrogen, halogen, hydroxyl, cyano, C₁-C₄ alkyl, C₁-C₄ halogenoalkyl, C₁-C₄-alkoxy, C₃-C₆ cycloalkyl, —NH₂, —NH(C₁-C₄ alkyl), and —N(C₁-C₄ alkyl)₂; and
Q is selected from the group consisting of 6- or 10 membered aryl optionally substituted with 1, 2 or 3 substituents independently selected from the group consisting of halogen, cyano, nitro, hydroxyl, C₁-C₄ alkyl, C₁-C₄ halogenoalkyl, C₁-C₄ alkoxy, C₃-C₆ cycloalkyl, —NH₂, —NH(C₁-C₄ alkyl), —N(C₁-C₄ alkyl)₂, —NH(C₃-C₆ cycloalkyl), —N(C₁-C₄ alkyl)(C₃-C₆-cycloalkyl), —NHSO₂(C₁-C₄ alkyl), —SC₁-C₄ alkyl, —S(O)C₁-C₄ alkyl, —SO₂C₁-C₄ alkyl, —S(O)C₁-C₄-halogenoalkyl and —SO₂C₁-C₄ halogenoalkyl, wherein the 6- or 10 membered aryl is optionally fused with a 4- to 7-membered heterocycloalkyl having 1 or 2 heteroatoms selected from the group O, S, and N and wherein the carbons of the heterocycloalkyl are optionally substituted with 1, 2 or 3 substituents independently selected from the group halogen, cyano, nitro, hydroxyl, oxo, C₁-C₄ alkyl, C₃-C₆ cycloalkyl, C₁-C₄ halogenoalkyl, C₁-C₄ alkoxy, —NH₂, —NH(C₁-C₄ alkyl), and —N(C₁-C₄ alkyl)₂ and any N in the heterocycloalkyl is, valency permitting, substituted with a substituent selected from the group consisting of hydrogen, C₁-C₄ alkyl, and C₃-C₆ cycloalkyl; 5- to 10-membered heteroaryl having 1 or 2 heteroatoms selected from the group O, S, and N and wherein the carbons of the 5- to 10-membered heteroaryl are optionally substituted with 1, 2 or 3 substituents independently selected from the group consisting of halogen, cyano, nitro, hydroxyl, C₁-C₄ alkyl, C₃-C₆ cycloalkyl, C₁-C₄ halogenoalkyl, C₁-C₄ alkoxy, —NH₂, —NH(C₁-C₄ alkyl), and —N(C₁-C₄ alkyl)₂ and any N in the heteroaryl, valency permitting, is optionally substituted with a substituent selected from the group consisting of hydrogen, C₁-C₄ alkyl, and C₃-C₆ cycloalkyl; a 4- to 7-membered heterocycloalkyl having 1 or 2 heteroatoms selected from the group O, S, N, wherein the heterocycloalkyl is optionally benzo-fused, wherein the carbons of the 4- to 7-membered heterocycloalkyl or optionally benzo-fused 4- to 7-membered heterocycloalkyl are optionally substituted with 1, 2, 3, or 4 substituents independently selected from the group consisting of halogen, cyano, nitro, hydroxyl, oxo, C₁-C₄ alkyl, C₃-C₆ cycloalkyl, C₁-C₄ halogenoalkyl, C₁-C₄ alkoxy, —NH₂, —NH(C₁-C₄ alkyl), and —N(C₁-C₄ alkyl)₂ and any N in the heterocycloalkyl is optionally substituted with a substituent selected from the group consisting of hydrogen, C₁-C₄ alkyl, and C₃-C₆ cycloalkyl; 6- or 10 membered aryloxy optionally substituted with 1, 2 or 3 substituents independently selected from the group consisting of halogen, cyano, nitro, hydroxyl, C₁-C₄ alkyl, C₃-C₆ cycloalkyl, C₁-C₄ halogenoalkyl, C₁-C₄ alkoxy, —NH₂, —NH(C₁-C₄ alkyl), —N(C₁-C₄ alkyl)₂, —NH(C₃-C₆ cycloalkyl), —N(C₁-C₄ alkyl)(C₃-C₆-cycloalkyl), —NHSO₂(C₁-C₄ alkyl), —SC₁-C₄ alkyl, —S(O)C₁-C₄ alkyl, —SO₂C₁-C₄ alkyl, —S(O)C₁-C₄-halogenoalkyl and —SO₂C₁-C₄ halogenoalkyl; 6- or 10 membered arylthio-oxy optionally substituted with 1, 2 or 3 substituents independently selected from the group consisting of halogen, cyano, nitro, hydroxyl, C₁-C₄ alkyl, C₃-C₆ cycloalkyl, C₁-C₄ halogenoalkyl, C₁-C₄ alkoxy, —NH₂, —NH(C₁-C₄ alkyl), —N(C₁-C₄ alkyl)₂, —NH(C₃-C₆ cycloalkyl), —N(C₁-C₄ alkyl)(C₃-C₆-cycloalkyl), —NHSO₂(C₁-C₄ alkyl), —SC₁-C₄ alkyl, —S(O)C₁-C₄ alkyl, —SO₂C₁-C₄ alkyl, —S(O)C₁-C₄-halogenoalkyl and —SO₂C₁-C₄ halogenoalkyl; and 5- to 10-membered heteroaryloxy optionally substituted with 1, 2 or 3 substituents independently selected from the group consisting of halogen, cyano, nitro, hydroxyl, oxo, C₁-C₄ alkyl, C₃-C₆ cycloalkyl, C₁-C₄ halogenoalkyl, C₁-C₄ alkoxy, —NH₂, —NH(C₁-C₄ alkyl), —N(C₁-C₄ alkyl)₂, —NH(C₃-C₆ cycloalkyl), —N(C₁-C₄ alkyl)(C₃-C₆-cycloalkyl), —NHSO₂(C₁-C₄ alkyl), —SC₁-C₄ alkyl, —S(O)C₁-C₄ alkyl, —SO₂C₁-C₄ alkyl, —S(O)C₁-C₄-halogenoalkyl and —SO₂C₁-C₄ halogenoalkyl;
excluding the compounds: N-[(4S)-chroman-4-yl]-8-(3,5-dichlorophenyl)-4-(dimethylamino)-1,7-naphthyridine-3-carboxamide;
N-[(4S)-chroman-4-yl]-8-(2,3-dichlorophenyl)-4-(dimethylamino)-1,7-naphthyridine-3-carboxamide;
N-[(4S)-chroman-4-yl]-8-(3,5-dichlorophenyl)-4-(morpholin-4-yl)-1,7-naphthyridine-3-carboxamide;
N-[(4S)-chroman-4-yl]-8-(2,3-dichlorophenyl)-4-(morpholin-4-yl)-1,7-naphthyridine-3-carboxamide;
4-chloro-N-[(4S)-chroman-4-yl]-8-(2,3-dichlorophenyl)-1,6-naphthyridine-3-carboxamide;
8-bromo-N-[(4S)-chroman-4-yl]-4-oxo-1,4-dihydro-1,6-naphthyridine-3-carboxamide;
N-[(4S)-chroman-4-yl]-8-(3,5-dichlorophenyl)-4-oxo-1,4-dihydro-1,6-naphthyridine-3-carboxamide;
N-[(4S)-chroman-4-yl]-8-(2,3-dichlorophenyl)-4-oxo-1,4-dihydro-1,6-naphthyridine-3-carboxamide;
4-chloro-N-[(4S)-chroman-4-yl]-8-(3,5-dichlorophenyl)-1,6-naphthyridine-3-carboxamide;
4-chloro-N-[(4S)-chroman-4-yl]-8-(2,3-dichlorophenyl)-1,6-naphthyridine-3-carboxamide;
N-[(4S)-chroman-4-yl]-8-(3,5-dichlorophenyl)-4-(dimethylamino)-1,6-naphthyridine-3-carboxamide;
N-[(4S)-chroman-4-yl]-8-(2,3-dichlorophenyl)-4-(dimethylamino)-1,6-naphthyridine-3-carboxamide;
N-[(4S)-chroman-4-yl]-8-(2,3-dichlorophenyl)-4-(morpholin-4-yl)-1,6-naphthyridine-3-carboxamide;
N-[(4S)-chroman-4-yl]-8-(3,5-dichlorophenyl)-4-(morpholin-4-yl)-1,6-naphthyridine-3-carboxamide;
4-chloro-N-[(4S)-chroman-4-yl]-8-(3,5-dichlorophenyl)-1,5-naphthyridine-3-carboxamide;
4-chloro-N-[(4S)-chroman-4-yl]-8-(2,3-dichlorophenyl)-1,5-naphthyridine-3-carboxamide;
N-[(4S)-chroman-4-yl]-8-(3,5-dichlorophenyl)-4-(dimethylamino)-1,5-naphthyridine-3-carboxamide;
N-[(4S)-chroman-4-yl]-8-(3,5-dichlorophenyl)-4-(morpholin-4-yl)-1,5-naphthyridine-3-carboxamide;
N-[(4S)-chroman-4-yl]-8-(2,3-dichlorophenyl)-4-(dimethylamino)-1,5-naphthyridine-3-carboxamide;
N-[(4S)-chroman-4-yl]-8-(2,3-dichlorophenyl)-4-(morpholin-4-yl)-1,5-naphthyridine-3-carboxamide;
8-chloro-4-(3,5-dichlorophenyl)-N-[(4S)-chroman-4-yl]pyrido[3,2-d]pyrimidine-7-carboxamide;
4-(3,5-dichlorophenyl)-N-[(4S)-chroman-4-yl]-8-(dimethylamino)-pyrido[3,2-d]pyrimidine-7-carboxamide;
4-(3,5-dichlorophenyl)-N-[(4S)-chroman-4-yl]-8-(morpholin-4-yl)-pyrido[3,2-d]pyrimidine-7-carboxamide;
8-bromo-4-chloro-4-N-[(4S)-chroman-4-yl]cinnoline-3-carboxamide;
8-bromo-4-N-[(4S)-chroman-4-yl]-4-(dimethylamino)cinnoline-3-carboxamide;
8-bromo-4-N-[(4S)-chroman-4-yl]-4-methoxycinnoline-3-carboxamide;
8-(3,5-dichlorophenyl)-4-N-[(4S)-chroman-4-yl]-4-(dimethylamino)cinnoline-3-carboxamide;
8-(3-chlorophenyl)-4-N-[(4S)-chroman-4-yl]-4-(dimethylamino)cinnoline-3-carboxamide;
8-(2,3-dichlorophenyl)-4-N-[(4S)-chroman-4-yl]-4-(dimethylamino)cinnoline-3-carboxamide;
8-(3,4-difluorophenyl)-4-N-[(4S)-chroman-4-yl]-4-(dimethylamino)cinnoline-3-carboxamide;
8-(3,5-dichlorophenyl)-4-N-[(4S)-chroman-4-yl]-4-methoxycinnoline-3-carboxamide;
8-(3-chlorophenyl)-4-N-[(4S)-chroman-4-yl]-4-methoxycinnoline-3-carboxamide;
8-(2,3-dichlorophenyl)-4-N-[(4S)-chroman-4-yl]-4-methoxycinnoline-3-carboxamide;
8-(3,4-fluorophenyl)-4-N-[(4S)-chroman-4-yl]-4-methoxycinnoline-3-carboxamide;
8-chloro-4-(2,3-dichlorophenyl)-N-[(4S)-chroman-4-yl]pyrido[3,2-d]pyridazine-7-carboxamide;
4-(2,3-dichlorophenyl)-N-[(4S)-chroman-4-yl]-8-(dimethylamino)-pyrido[3,2-d]pyridazine-7-carboxamide;
4-(2,3-dichlorophenyl)-N-[(4S)-chroman-4-yl]-8-(morpholin-4-yl)-pyrido[3,2-d]pyridazine-7-carboxamide;
4-(3,5-dichlorophenyl)-N-[(4S)-chroman-4-yl]-8-(dimethylamino)-pyrido[3,2-d]pyridazine-7-carboxamide;
4-(3,5-dichlorophenyl)-N-[(4S)-chroman-4-yl]-8-(morpholin-4-yl)-pyrido[3,2-d]pyridazine-7-carboxamide;
N-[(4S)-chroman-4-yl]-8-(2,3,5-trifluorophenyl)-4-(morpholin-4-yl)-1,7-naphthyridine-3-carboxamide;
N-[(4S)-chroman-4-yl]-8-(2,3,5-trichlorophenyl)-4-(morpholin-4-yl)-1,7-naphthyridine-3-carboxamide;
N-[(4S)-chroman-4-yl]-8-(2,3-dichlorophenyl)-5-fluoro-4-(morpholin-4-yl)-1,7-naphthyridine-3-carboxamide;
N-[(4S)-chroman-4-yl]-8-(2,3,5-trifluorophenyl)-5-fluoro-4-(morpholin-4-yl)-1,7-naphthyridine-3-carboxamide;
N-[(4S)-chroman-4-yl]-8-(2,3,5-trifluorophenyl)-5-methoxy-4-(morpholin-4-yl)-1,7-naphthyridine-3-carboxamide;
N-[(4S)-chroman-4-yl]-8-(2,3-dichlorophenyl)-5-fluoro-4-(dimethylamino)-1,7-naphthyridine-3-carboxamide;
N-[(4S)-chroman-4-yl]-8-(2,3-dichlorophenyl)-5-methoxy-4-(dimethylamino)-1,7-naphthyridine-3-carboxamide;
N-[(4S)-chroman-4-yl]-8-(2,3,5-trifluorophenyl)-5-fluoro-4-(dimethylamino)-1,7-naphthyridine-3-carboxamide;
N-[(4S)-chroman-4-yl]-8-(2,3,5-trifluorophenyl)-5-methoxy-4-(dimethylamino)-1,7-naphthyridine-3-carboxamide;
N-[(4S)-chroman-4-yl]-8-(2,3-dichlorophenyl)-5-methoxy-4-(morpholin-4-yl)-1,7-naphthyridine-3-carboxamide;
N-[(4S)-chroman-4-yl]-8-(3,5-dichlorophenyl)-5-methoxy-4-(dimethylamino)-1,6-naphthyridine-3-carboxamide;
N-[(4S)-chroman-4-yl]-8-(2,3-dichlorophenyl)-7-methoxy-4-(morpholin-4-yl)-1,6-naphthyridine-3-carboxamide;
N-[(4S)-chroman-4-yl]-8-(2,3-dichlorophenyl)-7-methoxy-4-(dimethylamino)-1,6-naphthyridine-3-carboxamide;
N-[(4S)-chroman-4-yl]-8-(3,5-dichlorophenyl)-7-methoxy-4-(dimethylamino)-1,6-naphthyridine-3-carboxamide;
N-[(4S)-chroman-4-yl]-8-(3,5-dichlorophenyl)-7-methoxy-4-(morpholin-4-yl)-1,6-naphthyridine-3-carboxamide;
N-[(4S)-chroman-4-yl]-8-(3,5-dichlorophenyl)-5-methoxy-4-(morpholin-4-yl)-1,6-naphthyridine-3-carboxamide;
N-[(4S)-chroman-4-yl]-8-(2,3-dichlorophenyl)-5-methoxy-4-(morpholin-4-yl)-1,6-naphthyridine-3-carboxamide;
N-[(4S)-chroman-4-yl]-8-(2,3-dichlorophenyl)-5-methoxy-4-(dimethylamino)-1,6-naphthyridine-3-carboxamide;
7-chloro-N-[(4S)-chroman-4-yl]-8-(2,3,5-trifluorophenyl)-4-(dimethylamino)-1,5-naphthyridine-3-carboxamide;
7-chloro-N-[(4S)-chroman-4-yl]-8-(2,3,5-trifluorophenyl)-4-(morpholin-4-yl)-1,5-naphthyridine-3-carboxamide;
7-chloro-N-[(4S)-chroman-4-yl]-8-(3,5-dichlorophenyl)-4-(dimethylamino)-1,5-naphthyridine-3-carboxamide;
7-chloro-N-[(4S)-chroman-4-yl]-8-(2,3-dichlorophenyl)-4-(dimethylamino)-1,5-naphthyridine-3-carboxamide;
7-chloro-N-[(4S)-chroman-4-yl]-8-(2,3-dichlorophenyl)-4-(morpholin-4-yl)-1,5-naphthyridine-3-carboxamide;
7-methoxy-N-[(4S)-chroman-4-yl]-8-(3,5-dichlorophenyl)-4-(dimethylamino)-1,5-naphthyridine-3-carboxamide;
7-fluoro-N-[(4S)-chroman-4-yl]-8-(3,5-dichlorophenyl)-4-(dimethylamino)-1,5-naphthyridine-3-carboxamide;
7-methoxy-N-[(4S)-chroman-4-yl]-8-(2,3-dichlorophenyl)-4-(dimethylamino)-1,5-naphthyridine-3-carboxamide;
7-methoxy-N-[(4S)-chroman-4-yl]-8-(2,3-dichlorophenyl)-4-(morpholin-4-yl)-1,5-naphthyridine-3-carboxamide;
7-methoxy-N-[(4S)-chroman-4-yl]-8-(2,3,5-trifluorophenyl)-4-(dimethylamino)-1,5-naphthyridine-3-carboxamide;
7-methoxy-N-[(4S)-chroman-4-yl]-8-(2,3,5-trifluorophenyl)-4-(morpholin-4-yl)-1,5-naphthyridine-3-carboxamide;
7-fluoro-N-[(4S)-chroman-4-yl]-8-(2,3-dichlorophenyl)-4-(dimethylamino)-1,5-naphthyridine-3-carboxamide;
7-fluoro-N-[(4S)-chroman-4-yl]-8-(2,3-dichlorophenyl)-4-(morpholin-4-yl)-1,5-naphthyridine-3-carboxamide;
7-fluoro-N-[(4S)-chroman-4-yl]-8-(2,3,5-trifluorophenyl)-4-(dimethylamino)-1,5-naphthyridine-3-carboxamide; and
7-fluoro-N-[(4S)-chroman-4-yl]-8-(2,3,5-trifluorophenyl)-4-(morpholin-4-yl)-1,5-naphthyridine-3-carboxamide;
   or a salt thereof.

In one embodiment, the present invention also provides compositions, comprising: a compound of formula (I) or a salt thereof and an acceptable excipient, the composition optionally further comprising at least one additional active compound.

In one embodiment, the present invention also provides a method for treating parasites, comprising: administering to a subject in need thereof an effective amount of a compound of formula (I) or a salt thereof, the method optionally further comprising an effective amount of at least one additional active compound.

In one embodiment, the present invention also provides a method for controlling parasites, comprising: administering to a subject in need thereof an effective amount of a compound of formula (I) or a salt thereof, the method optionally further comprising an effective amount of at least one additional active compound.

In one embodiment, the present invention also provides a method for treating or controlling parasites, comprising: contacting a subject's environment with an effective amount of a compound of formula (I) or a salt thereof, the method optionally further comprising an effective amount of at least one additional active compound.

Thus, the invention provides for the use of the compounds of the invention as a medicament, including for the manufacture of a medicament. In one embodiment, the invention provides the manufacture of a medicament comprising a compound of formula (I) or a salt thereof for treating parasites. In one embodiment, the invention provides the manufacture of a medicament comprising a compound of formula (I) or a salt thereof for controlling parasites.

The present invention also provides processes from making compounds of the invention and intermediates thereof.

### DETAILED DESCRIPTION

The term “C₁-C₄ alkyl” refers to a straight or branched alkyl chain having from one to four carbon atoms and includes methyl, ethyl, propyl, isopropyl, butyl, and the like.

The term “C₁-C₄ halogenoalkyl” refers to a straight or branched alkyl chain having from one to four carbon atoms and 1 to 5 halogen and includes fluoromethyl, difluoromethyl, trifluoromethyl, 2,2,2-trifluoroethyl, 1,2,2-trifluoroethyl, 3,3,3-trifluoropropyl, and the like.

The term “C₂-C₄ alkenyl” refers to a straight or branched alkenyl chain having from two to four carbon atoms and one carbon-carbon double bond, and includes ethylene, propylene, iso-propylene, butylene, iso-butylene, sec-butylene, and the like.

The term “C₂-C₄ alkynyl” refers to a straight or branched alkynyl chain having from two to four carbon atoms and one carbon-carbon triple bond, and includes acetylene, propargyl, and the like.

The term “C₁-C₄ alkoxy” refers to a C₁-C₄ alkyl attached through an oxygen atom and includes methoxy, ethoxy, propoxy, isopropoxy, butoxy, and the like.

The term “C₃-C₆ cycloalkyl” refers to an alkyl ring of three to six carbon atoms, and includes cyclopropyl, cyclobutyl, cyclopentyl, and cyclohexyl.

The terms “halogen” and “halogeno” refers to a chloro, fluoro, bromo or iodo atom.

The term “C₆- or C₁₀-membered aryl” refers to phenyl or naphthyl.

The term “C₆- or C₁₀-membered aryloxy” refers to phenyl or naphthyl attached through an oxygen atom and includes phenoxy and naphtyloxy.

The term “C₆- or C₁₀-membered arylthio-oxy” refers to phenyl or naphthyl attached through an sulfur atom and includes phenthio-oxy and naphtylthio-oxy. Further it is understood that the term “C₆- or C₁₀-membered arylthio-oxy” also encompasses in which the sulfur is the —SO₂— and —S(O)—.

The term “4- to 7-membered heterocycloalkyl” refers to a 4 to 7 membered monocyclic saturated or partially (but not fully) unsaturated ring having one or two heteroatoms selected from the group consisting of nitrogen, oxygen, and sulfur and the ring optionally includes a carbonyl to form a lactam or lactone. It is understood that where sulfur is included that the sulfur may be either —S—, —SO—, or —SO₂—. For example, but not limiting, the term includes azetidinyl, pyrrolidinyl, piperidinyl, piperazinyl, morpholinyl, thiomorpholinyl, oxetanyl, dioxolanyl, tetrahydropyranyl, tetrahydrothiopyranyl, tetrahydrofuryl, hexahydropyrimidinyl, tetrahydropyrimidinyl, dihydroimidazolyl, and the like.

The term “5-membered heteroaryl” refers to a five membered, monocyclic, fully unsaturated, ring with one to four carbon atoms and one to four heteroatoms selected from the group consisting of nitrogen, oxygen, and sulfur. For example, but not limiting, the term includes furyl, thienyl, pyrrolyl, imidazolyl, isothiazolyl, isoxazolyl, oxadiazolyl, oxazolyl, thiazolyl, thiadiazolyl, triazolyl, tetrazolyl, and the like. It is understood that a 5-membered heteroaryl can be attached as a substituent through a ring carbon or a ring nitrogen atom where such an attachment mode is available, for example for a pyrrolyl, imidazolyl, pyrazolyl, triazolyl, and the like.

The term “6-membered heteroaryl” refers to a six membered, monocyclic, fully unsaturated ring with one to five carbon atoms and one or more, typically one to four, heteroatoms selected from the group consisting of nitrogen, oxygen, and sulfur. For example, but not limiting, the term includes pyrazinyl, pyrazolyl, pyridazinyl, pyridyl, pyrimidyl, and the like. It is understood that a 6-membered heteroaryl can be attached as a substituent through a ring carbon or a ring nitrogen atom where such an attachment mode is available.

The term “5- to 10-membered heteroaryl having 1 or 2 heteroatoms selected from the group O, S, and N” refers to a five to ten membered, monocyclic or polycyclic fully unsaturated, ring or ring system with one to nine carbon atoms and one or two heteroatoms selected from the group consisting of nitrogen, oxygen, and sulfur. For example, but not limiting, the term includes furyl, thienyl, pyrrolyl, imidazolyl, isothiazolyl, isoxazolyl, oxadiazolyl, oxazolyl, thiazolyl, pyrazinyl, pyrazolyl, pyridazinyl, pyridyl, pyrimidyl, azepinyl, diazepinyl, benzofuryl, benzothienyl, indolyl, isoindolyl, benzimidazolyl, benzisothiazolyl, benzisoxazolyl, benzoxazolyl, benzopyrazinyl, benzopyrazolyl, quinazolyl, thienopyridyl, quinolyl, isoquinolyl benzothiazolyl, and the like. It is understood that a 5- to 10-membered heteroaryl having 1 or 2 heteroatoms selected from the group O, S, and N can be attached as a substituent through a ring carbon or a ring nitrogen atom where such an attachment mode is available.

The term “5- to 10-membered heteroaryloxy” refers to a 5- to 10-membered heteroaryl having 1 or 2 heteroatoms selected from the group O, S, and N attached through an oxygen atom and includes imidazolyloxy, pyrazolyloxy, pyridyloxy, pyrimidyloxy, quinolyloxy, and the like

The term “oxo” refers to an oxygen atom doubly bonded to the carbon to which it is attached to form the carbonyl of a ketone or aldehyde. For example, a pryidone radical is contemplated as an oxo substituted 6-membered heteroaryl.

The term “carboxyl” refers to the group below:

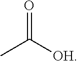

The term “carbamoyl” refers the group below:

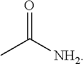

The term “C₁-C₄ alkoxy carbonyl” refers the group below:

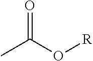

wherein R is a C₁-C₄ alkyl.

The term “nil” as used herein with reference to a group, substituent, moiety, or the like, indicates that that group, substituent, or moiety is not present. Wherein a group, substituent, or moiety is ordinarily bonded to two or more other groups, substituents, or moieties, the others are bonded together in lieu of the group, substituent, or moiety which is nil. For example, with a compound having the structure A-B—C; wherein B is nil, then A is directly bonded to C and the compound is A-C. As another example, with a compound having the structure A-B—C; wherein C is nil, then the compound is A-B.

The term “salt” refers to salts of veterinary or pharmaceutically acceptable organic acids and bases or inorganic acids and bases. Such salts are well known in the art and include those described in Journal of Pharmaceutical Science, 66, 2-19 (1977). An example is the hydrochloride salt.

The term “substituted,” including when used in “optionally substituted” refers to one or more hydrogen radicals of a group being replaced with non-hydrogen radicals (substituent(s)). It is understood that the substituents may be either the same or different at every substituted position. Combinations of groups and substituents envisioned by this invention are those that are stable or chemically feasible.

The term “stable” refers to compounds that are not substantially altered when subjected to conditions to allow for their production. In a non-limiting example, a stable compound or chemically feasible compound is one that is not substantially altered when kept at a temperature of 40° C. or less, in the absence of moisture or other chemically reactive conditions, for about a week.

It is understood that, where the terms defined herein mention a number of carbon atoms, that the mentioned number refers to the mentioned group and does not include any carbons that may be present in any optional substituent(s) thereon or any carbons that may be present as part of a fused ring, including a benzo-fused ring.

The skilled artisan will appreciate that certain of the compounds of the present invention exist as isomers. All stereoisomers of the compounds of the invention, including geometric isomers, enantiomers, and diastereomers, in any ratio, are contemplated to be within the scope of the present invention.

The skilled artisan will also appreciate that certain of the compounds of the present invention exist as tautomers. All tautomeric forms the compounds of the invention are contemplated to be within the scope of the present invention.

Compounds of the invention also include all isotopic variations, in which at least one atom of the predominant atom mass is replaced by an atom having the same atomic number, but an atomic mass different from the predominant atomic mass. Use of isotopic variations (e.g., deuterium, ²H) may afford greater metabolic stability. Additionally, certain isotopic variations of the compounds of the invention may incorporate a radioactive isotope (e.g., tritium, ³H, or ¹⁴C), which may be useful in drug and/or substrate tissue distribution studies. Substitution with positron emitting isotopes, such as ¹¹C, ¹⁸F, ¹⁵O and ¹³N, may be useful in Positron Emission Topography (PET) studies.

The terms “compounds of the invention” and “a compound of the invention” and “compounds of the present invention” and a like include the embodiment of formula (I) and the other more particular embodiments encompassed by formula (I) described herein and the exemplified compounds described herein and a salt of each of these embodiments.

It is understood that the compounds wherein either one of Z₁ and Z₄ is O or S, Z₂ is nil when Z₁ is O or S, and Z₃ is nil when Z₄ is O or S give rise to the compounds in which a thienyl or furyl ring is fused to the ring containing Y₁ and Y₂.

The compound of formula (I) with G as defined has the formula:

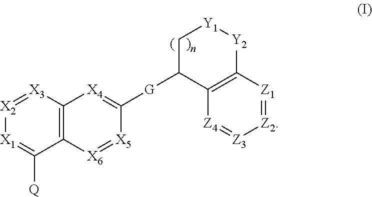

Further embodiments of compounds of the invention are provided below:
(a) One embodiment relates to a compound of formula (I).
(d) One embodiment relates to compounds of formula (I) wherein X₁ is N; X₂ is CR₂; X₃ is CR₃; X₄ is CR₄; X₅ is CR₅; and X₆ is N; or a salt thereof.
(e) One embodiment relates to compounds of formula (I) wherein X₁ is CR₁; X₂ is CR₂; X₃ is CR₃; X₄ is CR₄; X₅ is N; and X₆ is CR₆; or a salt thereof.
(f) One embodiment relates to compounds of formula (I) wherein X₁ is CR₁; X₂ is CR₂; X₃ is CR₃; X₄ is N; X₅ is N; and X₆ is N; or a salt thereof.
(g) One embodiment relates to embodiments (a), (d), (e) and (f) wherein Q is a 6- or 10 membered aryl optionally substituted with 1, 2 or 3 substituents independently selected from the group consisting of halogen, cyano, nitro, hydroxyl, C₁-C₄ alkyl, C₁-C₄ halogenoalkyl, C₁-C₄ alkoxy, C₃-C₆ cycloalkyl, —NH₂, —NH(C₁-C₄ alkyl), —N(C₁-C₄ alkyl)₂, —NH(C₃-C₆ cycloalkyl), —N(C₁-C₄ alkyl)(C₃-C₆-cycloalkyl), —NHSO₂(C₁-C₄ alkyl), —SC₁-C₄ alkyl, —S(O)C₁-C₄ alkyl, —SO₂C₁-C₄ alkyl, —S(O)C₁-C₄-halogenoalkyl and —SO₂C₁-C₄ halogenoalkyl; or a salt thereof.
(h) One embodiment relates to embodiments (a), (d), (e) and (f) wherein Q is 6-membered aryl optionally substituted with 1, 2 or 3 substituents independently selected from the group consisting of halogen, cyano, nitro, hydroxyl, C₁-C₄ alkyl, C₁-C₄ halogenoalkyl, C₁-C₄ alkoxy, C₃-C₆ cycloalkyl, —NH₂, —NH(C₁-C₄ alkyl), —N(C₁-C₄ alkyl)₂, —NH(C₃-C₆ cycloalkyl), —N(C₁-C₄ alkyl)(C₃-C₆-cycloalkyl), —NHSO₂(C₁-C₄ alkyl), —SC₁-C₄ alkyl, —S(O)C₁-C₄ alkyl, —SO₂C₁-C₄ alkyl, —S(O)C₁-C₄-halogenoalkyl and —SO₂C₁-C₄ halogenoalkyl, wherein the 6-membered aryl is fused with a 4- to 7-membered heterocycloalkyl having 1 or 2 heteroatoms selected from the group O, S, and N and wherein the carbons of the heterocycloalkyl are optionally substituted with 1, 2 or 3 substituents independently selected from the group consisting of halogen, cyano, nitro, hydroxyl, oxo, C₁-C₄ alkyl, C₃-C₆ cycloalkyl, C₁-C₄ halogenoalkyl, C₁-C₄ alkoxy, —NH₂, —NH(C₁-C₄ alkyl), and —N(C₁-C₄ alkyl)₂ and any N in the heterocycloalkyl is substituted with a substituent selected from the group consisting of hydrogen, C₁-C₄ alkyl, and C₃-C₆ cycloalkyl; or a salt thereof.
(i) One embodiment relates to embodiments (a), (d), (e) and (f) wherein Q is a 5- to 10-membered heteroaryl having 1 or 2 heteroatoms selected from the group O, S, and N and wherein the carbons of the heteroaryl are optionally substituted with 1, 2 or 3 substituents independently selected from the group consisting of halogen, cyano, nitro, —OH, C₁-C₄ alkyl, C₃-C₆ cycloalkyl, C₁-C₄ halogenoalkyl, C₁-C₄ alkoxy, —NH₂, —NH(C₁-C₄ alkyl), and —N(C₁-C₄ alkyl)₂ and any N in the heteroaryl is optionally substituted with a substituent selected from the group consisting of hydrogen, C₁-C₄ alkyl, and C₃-C₆ cycloalkyl; or a salt thereof.
(j) One embodiment relates to embodiments (a), (d), (e) and (f) wherein Q is a 4- to 7-membered heterocycloalkyl having 1 or 2 heteroatoms selected from the group O, S, N, wherein the heterocycloalkyl is optionally benzo-fused, wherein the carbons of the heterocycloalkyl or optionally benzo-fused heterocycloalkyl are optionally substituted with 1, 2, 3, or 4 substituents independently selected from the group consisting of halogen, cyano, nitro, hydroxyl, oxo, C₁-C₄ alkyl, C₃-C₆ cycloalkyl, C₁-C₄ halogenoalkyl, C₁-C₄ alkoxy, —NH₂, —NH(C₁-C₄ alkyl), and —N(C₁-C₄ alkyl)₂ and any N in the heterocycloalkyl is optionally substituted with a substituent selected from the group consisting of hydrogen, C₁-C₄ alkyl, and C₃-C₆ cycloalkyl; or a salt thereof.
(k) One embodiment relates to embodiments (a), (b), (c), (d), (e) and (f) wherein Q is a 6- or 10 membered aryloxy optionally substituted with 1, 2 or 3 substituents independently selected from the group consisting of halogen, cyano, nitro, hydroxyl, C₁-C₄ alkyl, C₃-C₆ cycloalkyl, C₁-C₄ halogenoalkyl, C₁-C₄ alkoxy, —NH₂, —NH(C₁-C₄ alkyl), —N(C₁-C₄ alkyl)₂, —NH(C₃-C₆ cycloalkyl), —N(C₁-C₄ alkyl)(C₃-C₆-cycloalkyl), —NHSO₂(C₁-C₄ alkyl), —SC₁-C₄ alkyl, —S(O)C₁-C₄ alkyl, —SO₂C₁-C₄ alkyl, —S(O)C₁-C₄-halogenoalkyl and —SO₂C₁-C₄ halogenoalkyl; or a salt thereof.
(l) One embodiment relates to embodiments (a), (d), (e) and (f) wherein Q is a and 5- to 10-membered heteroaryloxy optionally substituted with 1, 2 or 3 substituents independently selected from the group consisting of halogen, cyano, nitro, hydroxyl, oxo, C₁-C₄ alkyl, C₃-C₆ cycloalkyl, C₁-C₄ halogenoalkyl, C₁-C₄ alkoxy, —NH₂, —NH(C₁-C₄ alkyl), —N(C₁-C₄ alkyl)₂, —NH(C₃-C₆ cycloalkyl), —N(C₁-C₄ alkyl)(C₃-C₆-cycloalkyl), —NHSO₂(C₁-C₄ alkyl), —SC₁-C₄ alkyl, —S(O)C₁-C₄ alkyl, —SO₂C₁-C₄ alkyl, —S(O)C₁-C₄-halogenoalkyl and —SO₂C₁-C₄ halogenoalkyl; or a salt thereof.
(m) One embodiment relates to embodiments (a), (d), (e), (f), (g), (h), (i), (j), (k), and (1) wherein n is 1; or a salt thereof.
(n) One embodiment relates to embodiments (a), (d), (e), (f), (g), (h), (i), (j), (k), (1), and (m) wherein Y₁ is CR₈R₉ and Y₂ is O; or a salt thereof;
(o) One embodiment relates to embodiments (a), (d), (e), (f), (g), (h), (i), (j), (k), (1), (m), and (n) wherein Z₁ is CR₁₁, Z₂ is CR₁₁, Z₃ is CR₁₁, and Z₄ is CR₁₁; or a salt thereof.
(p) One embodiment relates to embodiments (a), (d), (e), (f), (g), (h), (i), (j), (k), (1), (m), and (n) wherein Z₁ is CR₁₁, Z₂ is CR₁₁, Z₃ is nil, and Z₄ is S; or a salt thereof.
(q) One embodiment relates to embodiments (a), (d), (e), (f), (g), (h), (i), (j), (k), (1), (m), (n), (o), and (p) wherein R₄ is selected from the group consisting of C₁-C₄ alkyl, C₃-C₆ cycloalkyl, —N(C₁-C₄ alkyl)₂, and 4- to 7-membered heterocycloalkyl; or a salt thereof.
(r) One embodiment relates to embodiments (a), (d), (e), (f), (g), (h), (i), (j), (k), (1), (m), (n), (o), and (p) wherein R₄ is —N(C₁-C₄ alkyl)₂; or a salt thereof.
(s) Another embodiment relates to a salt of each of the exemplified compounds.

The compounds of the invention can be prepared by a variety of procedures, some of which are described below. All substituents, unless otherwise indicated, are as previously defined.

The products of each step can be recovered by conventional methods including extraction, evaporation, precipitation, chromatography, filtration, trituration, crystallization, and the like. The procedures may require protection of certain groups, for example hydroxyl, thiol, amino, or carboxyl groups to minimize unwanted reactions. The selection, use, and removal of protecting groups are well known and appreciated as standard practice, for example T. W. Greene and P. G. M. Wuts in Protective Groups in Organic Chemistry (John Wiley and Sons, 1991).

As used herein: AcOH refers to acetic acid; aq. refers to aqueous, br refers to broad, CH₃CN refers to acetonitrile, CH₂Cl₂ refers to methylene chloride, d refers to doublet,

dd refers to doublet of doublet, DIPEA refers to N-diisopropylethylamine, DMA refers to N,N-dimethylacetamide, DMF refers to N,N-dimethylformamide, DMSO refers to dimethylsulfoxide, ee: refers to enantiomeric excess, eq. refers to equivalent, ES refers to electrospray ionization, EtOAc refers to ethyl acetate, EtOH refers to ethanol, HATU refers to 1-[bis(dimethylamino)methylene]-1H-1,2,3-triazolo[4,5-b]pyridinium 3-oxide hexafluorophosphate, HPLC refers to high performance liquid chromatography, iPrOH refers to isopropanol, J refers to coupling constant, KOAc refers to potassium acetate, K₂CO₃ refers to potassium carbonate, LCMS refers to liquid chromatography-mass spectrometry, m/z: refers to mass-to-charge ratio, M refers to molarity, m refers to multiplet, MeOH refers to methanol, min. refers to minutes, NaHCO₃ refers to sodium bicarbonate, Na₂CO₃ refers to sodium carbonate, NEt₃ refers to triethylamine, NMR refers to nuclear magnetic resonance, NMP refers to N-methylpyrrolidone, q refers to quartet, rt refers to room temperature, Rₜ refers to retention time, s refers to singlet, sat. refers to saturated, T refers to temperature, t refers to triplet, dt refers to doublet of triplets, td refers to triplet of doublets, THF refers to tetrahydrofuran, wt refers to weight, and δ refers to chemical shift.

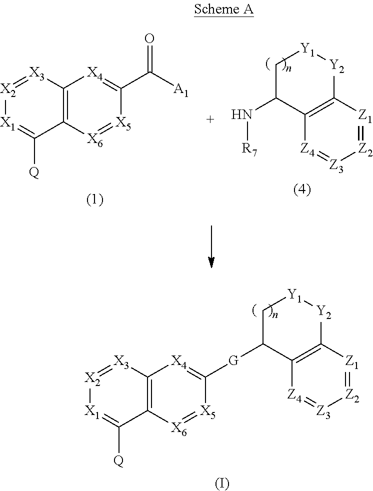

Scheme A depicts an amidation reaction of a compound of formula (1) and a compound of formula (2) to give a compound of formula (I). The depicted compound of formula (1) is one in which the group A₁ is a hydroxyl group, or an activating groups as is discussed below, and Q, X₁, X₂, X₃, X₄, X₅, and X₆ are as desired in the final compound of formula (I) or a group that gives rise to Q, X₁, X₂, X₃, X₄, X₅, and X₆ as desired in the final compound of formula (I). For example, a compound of formula (1) can be one in which the depicted group “Q” is a halogen which is further elaborated, in a subsequent step, not shown, to give a compound in which Q is as defined in formula (I). The preparation of such compounds of formula (1) is readily appreciated in the art. A compound of formula (2) is one in which R₇, n, Y₁, Y₂, Z₁, Z₂, Z₃, and Z₄ are as desired in the final product of formula (I) or a group that gives rise to R₇, Y₁, Y₂, Z₁, Z₂, Z₃, and Z₄ as desired in the final product of formula (I). The preparation of such compounds of formula (2) is readily appreciated in the art.

As mentioned above, Scheme A depicts the amidation of a compound of formula (1) using a compound of formula (2) to give a compound of formula (I). Typical groups A₁ are hydroxyl or a leaving group, such as chloro, bromo, or imidazolyl, an activating moiety, a mixed anhydride of another carboxylic acid, such as formic acid, acetic acid, or represents the other part of a symmetrical anhydride formed from two compounds of formula (1). For example, standard amide forming conditions can be used, such as those using coupling agents, including those used in peptide couplings, such as 2-(1H-7-azabenzotriazol-1-yl)-1,1,3,3-tetramethyl uronium hexafluorophosphate methanaminium (HATU), dicyclohexylcarbodiimide (DCC), and 1-(3-dimethylaminopropyl)-3-ethylcarbodiimide hydrochloride. If necessary or desired, an additive such as 4-(dimethylamino)pyridine, 1-hydroxybenzotriazole, and the like may be used to facilitate the reaction. Such reactions are generally carried out using a base, such as N-methylmorpholine or NEts, in a wide variety of suitable solvents such as CH₂Cl₂, DMF, NMP, DMA, THF, and the like. Such amide forming reactions are well understood and appreciated in the art.

It will be recognized by one of ordinary skill in the art that a compound of formula (I) can be elaborated in a variety of ways to give other compounds of formula (I). Such reactions include hydrolysis, oxidation, reduction, alkylation, arylation (including heteroaryl groups) amidations, sulfonations, and the like.

Also, in an optional step, not shown, the compounds of formula (I) can be converted to salts by methods well known and appreciated in the art.

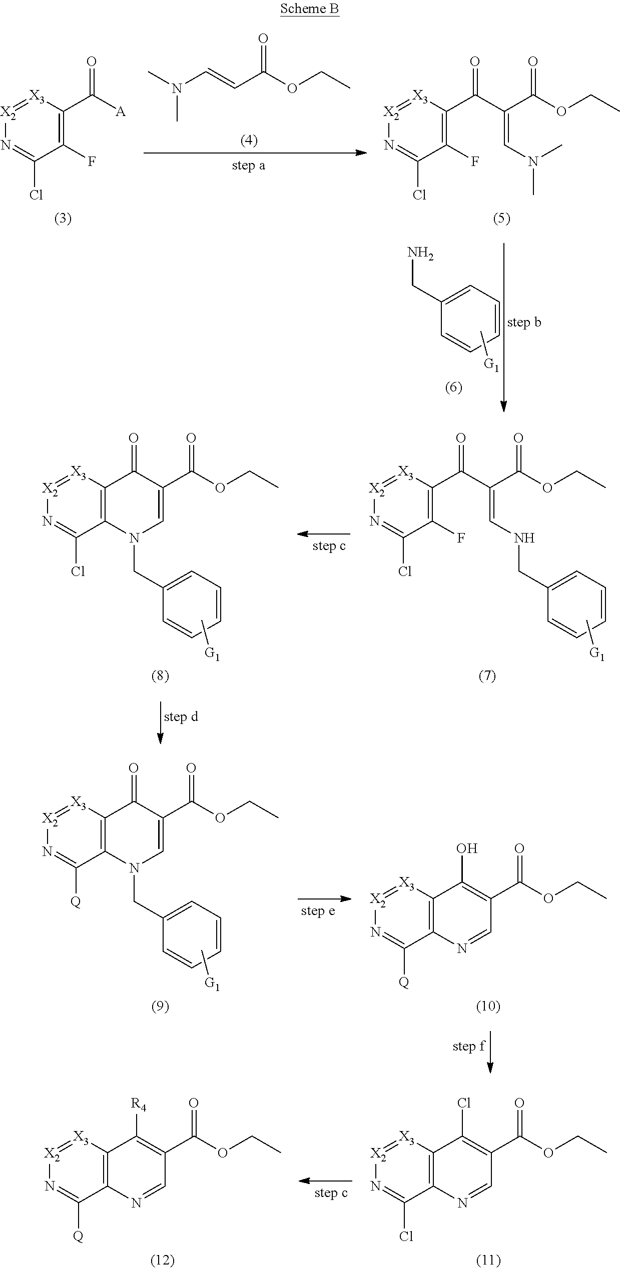

Scheme B depicts the preparation of compounds of formula (1) in which X₁ is N, X₄ is CR₄, X₅ is CR₅ wherein R₅ is hydrogen, and X₆ is N.

In Scheme B, step a, a compound of formula (3), is contacted with a compound of formula (4) to give a compound of formula (5). An compound of formula (3) is one in which X₂ and X₃ are as desired in the final compound of formula I or give rise to X₂ and X₃ as desired in the final compound of formula I. A compound of formula (4) can vary from the one depicted for example the depicted dimethyl amino group and can be other disubstituted amines, for example diethylamino or pyrolidin-1-yl and the ester can be other than the depicted ethyl, such as methyl or benzyl and other variations.

Such reactions are typically carried out in a solvent such as CH₂Cl₂, CH₃CN, THF or DMF, and the like and the reaction may be carried out using a suitable base, such as K₂CO₃, NaH, NEt₃ or DIPEA, and the like. The reaction is generally carried out using from 1 to 3 equivalents of a compound of formula (4). The reaction typically is carried out at temperatures of from 0° C. to 120° C. and requires about 0.5 hour to 1 day.

In Scheme B, step b, a compound of formula (5) is contacted with a compound of formula (6) to give a compound of formula (7). A compound of formula (6) is one in which G₁ is 1 to 3 substituents independently selected from the group halogen, nitro, C₁-C₄ alkyl, C₁-C₄-alkoxy. Such reactions are typically carried out in a solvent such as

Et₂O, EtOH, CH₃CN, THF or DMF, and the like. The reaction is generally carried out using from 1 to 3 equivalents of a compound of formula (6). The reaction typically is carried out at temperatures of from rt to 50° C. and require about 0.5 hour to 1 day.

In Scheme B, step c, a compound of formula (7) is cyclized to give a compound of formula (8). Such reactions are typically carried out in a solvent such as CH₃CN, THF or DMF and the like and the reaction may be carried out using a suitable base, such as K₂CO₃, NEt₃ or DIPEA, and the like. The reaction typically is carried out at temperatures of from rt to 100° C. and require about 1 hour to 1 day.

In Scheme B, step d, a compound of formula (8) is converted to a compound of formula (9). For example, a compound of formula (8) can undergo a variety of displacement reactions as well as amination, alkylation, alkoxylation, aryloxylation, arylthio-oxylation, heteroaryloxylation, and arylation, including heteroarylation, to give compound of formula (9). One particularly useful method uses boronic acid or boronic ester of the group Q. Such reactions are generally known as a Suzuki reaction and are well known in the art. While a Suzuki reaction is mentioned here in Scheme B it is understood that other carbon-carbon bond forming coupling reactions can be used with compounds of formula (8) to produce compounds of formula (9).

In Scheme B, step e, a compound of formula (9) is deprotected to give a compound of formula (10). Such deprotection reactions are well known in the art.

In Scheme B, step f, the R₄ hydroxyl of a compound of formula (10) is converted to a halogen, typically chloro, to give the depicted compound of formula (11). Such reactions are well known and utilize halogenation reagents such as thionyl chloride, thionyl bromide, phosphorous trichloride, phosphorous oxychloride, phosphorous tribromide, phosphorous oxybromide, phosphorous pentachloride, and phosphorous pentabromide. Such reactions are typically carried out in a solvent such as DMF, and the like. The reaction typically is carried out at temperatures of from rt to 50° C. and require about 0.5 hour to 1 day.

In Scheme B, step g, a compound of formula (11) in which R₄ is halogen, typically the depicted chloro, is elaborated to give a compound of formula (12) having other R₄ groups. Such reactions include, amination, alkoxylation, thioalkoxylation, carboxylation, alkylation, alkenylation, alkynylation, and arylation, and the like. Such reactions are well known in the art.

In Scheme B, in a step not depicted, the ester of a compound of formula (12) is hydrolyzed to give a compound of formula (1) in which A₁ is hydroxyl and X₁ is N, X₄ is CR₄, X₅ is CR₅ wherein R₅ is hydrogen, and X₆ is N.

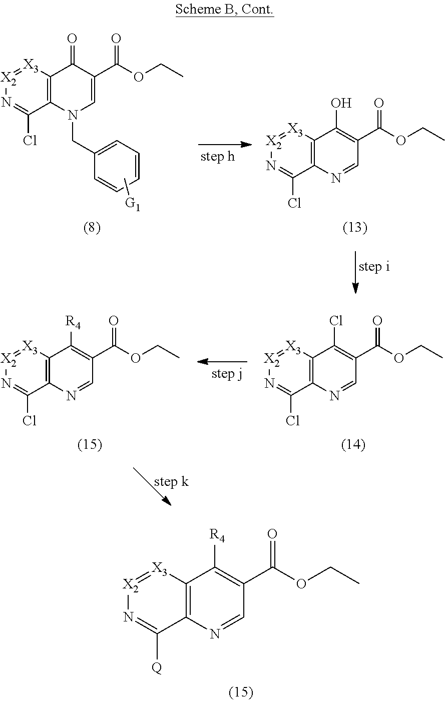

The order of some of the steps is not critical as depicted in Scheme B, continued above. In Scheme B, step h, a compound of formula (8) is deprotected as mentioned for step e above to give a compound of formula (13).

In Scheme B, step i, the R₄ hydroxyl of a compound of formula (13) is converted to a halogen, typically the depicted chloro, to give the depicted compound of formula (14) using the same methodology discussed above in step f.

In Scheme B, step j, a compound of formula (14) in which R₄ is halogen, typically the depicted chloro, is elaborated to give a compound of formula (15) having other R₄ groups as discussed above on step g.

In Scheme B, step k, a compound of formula (15) is converted to a compound of formula (1) using the methodology of step d above.

In Scheme B, continued, again in a step not depicted, the ester of a compound of formula (12) is hydrolyzed to give a compound of formula (1) in which A₁ is hydroxyl and X₁ is N, X₄ is CR₄, X₅ is CR₅ wherein R₅ is hydrogen, and X₆ is N.

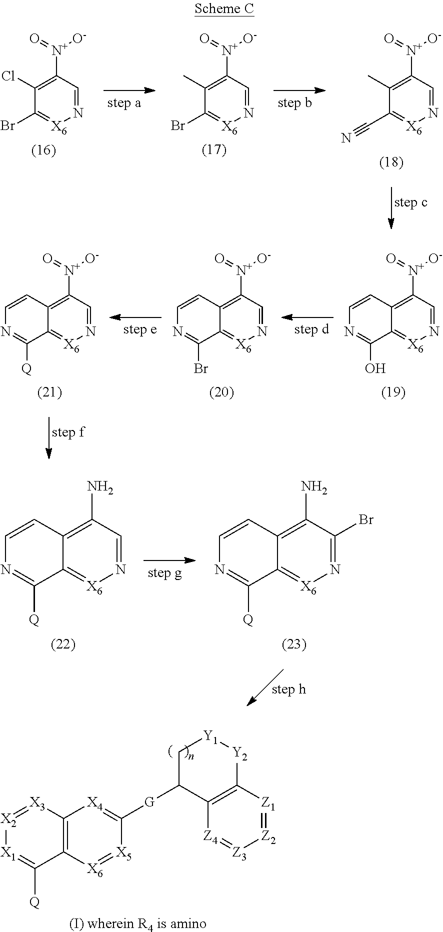

Scheme C depicts the preparation of compounds of formula (I) in which X₁ is N, X₂ is CR₂ wherein R₂ is hydrogen, X₃ is CR₃ wherein R₃ is hydrogen, X₄ is CR₄ wherein R₄ is amino or substituted amino, and X₅ is N.

In Scheme C, step a, a compound of formula (16) is converted to a compound of formula (17). Such conversions are readily accomplished by the use of organometallic reagents or the action of malonate diesters followed by hydrolysis and decarboxylation.

In Scheme C, step b, a compound of formula (17) gives a compound of formula (18). Such reaction are carried out using an alkali metal salt of cyanide, such as sodium cyanide or potassium cyanide and the like, and are typically carried out in a solvent such as 1,4-dioxane, MeOH, Toluene, CH₃CN, THF, DMF and the like. The reaction is generally carried out using from 1 to 2 equivalents of a compound of alkali metal cyanide. The reaction typically is carried out at temperatures of from rt to 150° C. and requires about 2 hour to 1 days.

In Scheme C, step c, a compound of formula (18) is homologated and cyclized to give a compound of formula (19). The homologation can be carried out using N,N-dimethylformamide dimethyl acetal or equivalents thereof to give a compound such as 4-[(E)-2-(dimethylamino) vinyl]-5-nitro-pyridine-3-carbonitrile (not shown). Such reactions are typically carried out in a solvent such as CH₂Cl₂, CH₃CN, MeOH, THF, DMF and the like. The reaction is generally carried out using from 1 to 4 equivalents of N,N-dimethylformamide dimethyl acetal. The reaction typically is carried out at temperatures of from rt to 60° C. and require about 1 hour to 1 days. The aminovinyl compound can then be cyclized under acidic conditions. Such cyclizations are typically carried out in a solvent such as acetic acid, and the like and the reaction may be carried out using a suitable acid, such as hydrobromic acid, sulfuric acid and the like. The reaction typically is carried out at temperatures of from rt to 150° C. and require about 0.5 hour to 6 hours.

In Scheme C, step d, a compound of formula (19) is halogenated using the methodology of Scheme B, step f, to give a compound of formula (20).

In Scheme C, step e, a compound of formula (20) is converted to a compound of formula (21) using the methodology of Scheme B, step d.

In Scheme C, step f, a compound of formula (21) is reduced to give a compound of formula (22). Such reduction of nitro groups to amines is well known in the art. For example, hydrogenation over catalysts or the action of reducing agents such as iron.

In Scheme C, step g, a compound of formula (22) is halogenated to give a compound of formula (23). Such halogenation reactions are well known using reagents such as N-bromosuccinimide, bromine, and the like.

In Scheme C, step h, a compound of formula (23) is converted to a compound of formula (I) in which R₄ is amino. Such carboxylative aminohomologations or amino cabonylations reactions are well known in the art. Such reactions are carried out using carbon dioxide and carbon monoxide, an amino of formula (2) and a variety of catalysts, such a palladium, molybdenum, and iron catalysts. The reactions are typically carried out in a solvent such as 1,4-dioxane, CH₃CN, THF, DMF, and the like. The reaction typically is carried out at temperatures of from rt to 150° C. and require about 2 hours to 1 day.

It is understood that a compound of formula (23) and a compound of formula (I) in which R₄ is amino can be elaborated into other amino containing compounds. For example, the amino group of a compound of formula (23) or a compound of formula (I) in which R₄ is amino can be alkylated or cyclized to give a heterocycloalkyl or can be sulfonated.

As above, in a step not shown, a compound of formula (I) can be converted to a salt thereof.

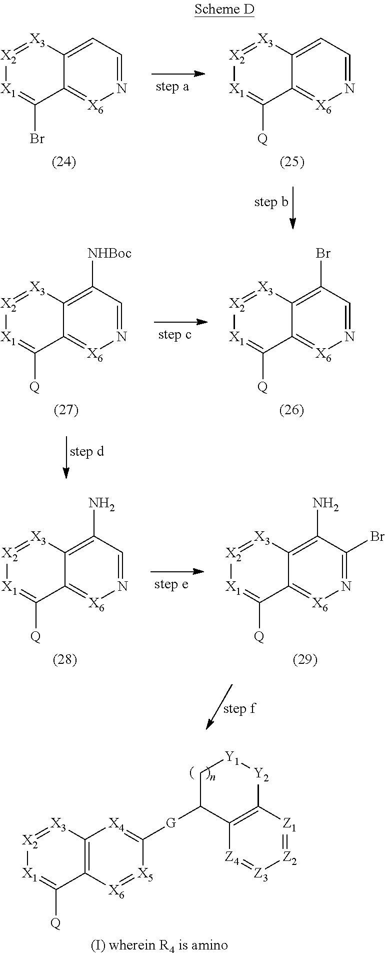

Scheme D depicts the preparation of compounds of formula (I) in which X₄ is CR₄ wherein R₄ is amino and X₅ is N.

In Scheme D, step a, a compound of formula (24) is converted to a compound of formula (25) using the methodology of Scheme B, step d.

In Scheme D, step b, a compound of formula (25) is converted to a compound of formula (26) using the methodology of Scheme C, step g, above.

In Scheme D, step c, a compound of formula (26) is aminocarboxylated to give a compound of formula (27). Such reactions are well known in the art are carried out using catalysts, such as palladium catalysts and tert-butyl carbamate and the like. The reaction is typically carried out using a solvent, such as 1,4-dioxane, THF, CH₂Cl₂, CH₃CN, MeOH, DMF, toluene and the like. The reaction is carried out using a base, such as Cs₂CO₃, NEt₃, K₂CO₃, KOAc, NaHCO₃, and the like. The reaction is carried out at temperatures of from 40° C. to 120° C. and typically require from 1 hour to 2 days.

In Scheme D, step d, depicts the deprotection of a compound of formula (27) to give a compound of formula (28). Such deprotections using acids are well known in the art.

In Scheme D, step e, compound of formula (28) is brominated using the methodology of Scheme C, step g, to give a compound of formula (29).

In Scheme D, step (f), a compound of formula (29) is converted to a compound of formula (I) in which R₄ is amino using the methodology of Scheme C, step h.

It is understood that a compound of formula (29) and a compound of formula (I) in which R₄ is amino can be elaborated into other amino containing compounds of formula (I).

As above, in an optional step not shown, a compound of formula (I) can be converted to a salt thereof.

The following examples are intended to be illustrative and non-limiting, and represent specific embodiments of the present invention.

Analyses methods A and B were performed using an Agilent 1200 Infinity Series Liquid Chromatography (LC) system, consisting of a 1260 HiP degasser (G4225A), 1260 Binary Pump (G1312B), 1290 auto-sampler (G4226A), 1290 thermo-stated column compartment (G1316C) and a 1260 Diode Array Detector (G4212B) coupled to an Agilent 6150 single quadrupole mass spectrometry (MS) detector. The injection volume was set to 1 μL by default. The UV (DAD) acquisition was performed at 40 Hz, with a scan range of 190-400 nm (by 5 nm step). A 1:1 flow split was used before the MS detector. The MS was operated with an electro-spray ionization source (ESI) in both positive & negative ion mode. The nebulizer pressure was set to 50 psi, the drying gas temperature and flow to 350° C. and 12 L/min respectively. The capillary voltages used were 4000V in positive mode and 3500V in negative mode. The MS acquisition range was set to 100-800 m/z with a step size of 0.2 m/z in both polarity modes. Fragmentor voltage was set to 70 (ESI+) or 120 (ESI−), Gain to 0.40 (ESI+) or 1.00 (ESI−) and the ion count threshold to 4000 (ESI+) or 1000 (ESI−). The overall MS scan cycle time was 0.15 s/cycle. Data acquisition was performed with Agilent Chemstation software.

Method A: Analyses were carried out on a Phenomenex Gemini-NX C18 column of 50 mm length, 2.1 mm internal diameter and 3 m particle size. The mobile phase used was: A1=Water with 0.1% formic acid/B1=CH₃CN with 0.1% formic acid. The run was performed at a temperature of 50° C. and a flow rate of 1.2 mL/min, with a gradient elution from 5% to 95% (B1) over 1.5 min followed by a 0.5 min hold at 95% (B1).

Method B: Analyses were carried out on a Waters XBridge C18 column of 50 mm length, 2.1 mm internal diameter and 3.5 m particle size. The mobile phase used was: A2=Water with 10 mM ammonium bicarbonate, adjusted at pH 9 with ammonium hydroxide/B2=CH₃CN. The run was performed at a temperature of 50° C. and a flow rate of 1.2 mL/min, with a gradient elution from 5% to 95% (B2) over 1.5 min followed by a 0.5 min hold at 95% (B2).

Analyses methods C and D were performed using a Waters Acquity UPLC Liquid Chromatography (LC) system, coupled to an Waters SQ Detector 2 single quadrupole mass spectrometry (MS) detector. The UV (DAD) acquisition was performed with a scan range of 200-400 nm (by 1.2 nm resolution). The MS was operated with an electro-spray ionization source (ESI) in both positive & negative ion mode. Capillary Voltage 3.50 (kV), Cone Voltage 35 (V), and Desolvation Temperature of 550° C. Desolvation gas flow 1000 L/Hr, Cone gas flow 50 L/Hr. The MS acquisition range was set to 100-1500 m/z. MS scan cycle time was 0.5 s. Data acquisition was performed with Waters Masslynx software.

Method C: Analyses were carried out on an Acquity UPLC BEH C18 column of 50 mm length, 2.1 mm internal diameter and 1.7 m particle size. The mobile phase used was: A1=Water with 0.1% formic acid/B1=CH₃CN with 0.1% formic acid. The injection volume was 0.1 μL. The run was performed at a temperature of 40° C. and a flow rate of 0.6 mL/min, with a gradient elution. Method info (Time (min) and B %): 0-5; 0.3-5; 2.5-95; 3.7-95; 4-5; 4.6-5.

Method D: Analyses were carried out on an Acquity UPLC BEH C18 column of 50 mm length, 2.1 mm internal diameter and 1.7 m particle size. The mobile phase used was: A1=Water with 10 mM ammonium acetate/B1=CH₃CN with 0.1% formic acid. The injection volume was 0.1 μL. The run was performed at a temperature of 45° C. and a flow rate of 0.5 mL/min, with a gradient elution. Method info (Time (min) and A %): 0-98; 0.3-98; 3.2-2; 4.4-2; 4.7-98.

### Example 1.1

### N-[(4S)-chroman-4-yl]-8-(3,5-dichlorophenyl)-4-(dimethylamino)-1,7-naphthyridine-3-carboxamide

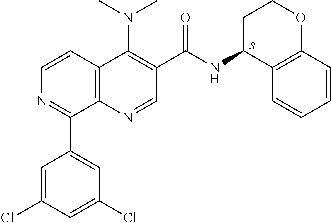

A mixture of 2-chloro-3-fluoro-pyridine-4-carboxylic acid (10.1 g, 56.3 mmol) and SOCl₂ (40 mL, 547 mmol) was heated at 80° C. for 2 hours. The reaction was allowed to cool to rt, and concentrated in vacuo. It was used directly in the next step: toluene (145 mL) and NEt₃ (9.8 mL, 70 mmol) were added followed by ethyl 3-(dimethylamino)-prop-2-enoate (10.2 g, 69.6 mmol). The reaction was heated at 80° C. and stirred for 45 min. The mixture was allowed to cool to rt, and filtered through Celite® (washing with EtOAc). The filtrate was concentrated in vacuo, and the residue was partitioned between EtOAc and aq. 2M HCl (150 mL of each). The layers were separated, and the aq. layer was extracted with EtOAc (150 mL). The combined organic layers were dried over anhydrous MgSO₄, filtered, and concentrated in vacuo to give ethyl 2-(2-chloro-3-fluoro-pyridine-4-carbonyl)-3-(dimethylamino)-prop-2-enoate. LCMS (method B): Rₜ=0.86 min, m/z=301.00 [M+H]⁺.

To a solution of ethyl 2-(2-chloro-3-fluoro-pyridine-4-carbonyl)-3-(dimethylamino)-prop-2-enoate (188 mg, 0.59 mmol) in Et₂O (2.4 mL) and EtOH (0.6 mL) was added 4-methoxybenzylamine (94 μL, 0.71 mmol). The reaction was stirred at rt for 15 min, forming a precipitate. The reaction mixture was concentrated in vacuo. The residue was triturated with cyclohexane to give ethyl 2-(2-chloro-3-fluoro-pyridine-4-carbonyl)-3-[(4-methoxyphenyl) methyl-amino]-prop-2-enoate. LCMS (method B): Rₜ=1.21 min, m/z=393 [M+H]⁺.

To a solution of ethyl 2-(2-chloro-3-fluoro-pyridine-4-carbonyl)-3-[(4-methoxyphenyl) methyl-amino]-prop-2-enoate (214 mg, 518 μmol) in DMF (2.6 mL) was added K₂CO₃ (230 mg, 1.66 mmol) at rt. The reaction mixture was heated at 40° C. and left to stir for 2 hours. After cooling down to rt, the reaction mixture was poured into ice water (20 mL), forming a fine precipitate. The precipitate was dissolved in EtOAc (20 mL), and the layers were separated. The aq. layer was extracted with EtOAc (2×10 mL), and the combined organic layers were washed with water (20 mL), dried over anhydrous MgSO₄, filtered, and concentrated in vacuo to give ethyl 8-chloro-1-[(4-methoxyphenyl) methyl]-4-oxo-1,7-naphthyridine-3-carboxylate. LCMS (method B): Rₜ=1.01 min, m/z=373 [M+H]⁺.

(3,5-Dichlorophenyl) boronic acid (110 mg, 0.56 mmol) was mixed with 1,1′-bis(diphenylphosphino) ferrocene-Pd(II)·CH₂Cl₂ complex and Na₂CO₃ (100 mg, 0.93 mmol). The vial was sealed, then evacuated and back-filled with N₂. Then, ethyl 8-chloro-1-[(4-methoxyphenyl)methyl]-4-oxo-1,7-naphthyridine-3-carboxylate (186 mg, 0.47 mmol) in 1,4-dioxane (2.4 mL, 28 mmol) was added, followed by water (0.8 mL, 40 mmol), and the reaction was heated at 100° C. in the microwave for 1 hour. The reaction mixture was filtered through Celite® (washing with EtOAc). The filtrate was washed with water (20 mL), dried over anhydrous MgSO₄, filtered, and concentrated in vacuo, then purified by column chromatography to give ethyl 8-(3,5-dichlorophenyl)-1-[(4-methoxyphenyl)methyl]-4-oxo-1,7-naphthyridine-3-carboxylate. LCMS (method B): Rₜ=1.30 min, m/z=483 [M+H]⁺.

To a solution of ethyl 8-(3,5-dichlorophenyl)-1-[(4-methoxyphenyl)methyl]-4-oxo-1,7-naphthyridine-3-carboxylate (877 mg, 1.72 mmol) in CH₂Cl₂ (9 mL) was added anisole (1 mL, 1.74 mmol), followed by TFA (2.5 mL, 33 mmol). The resulting reaction mixture was left to stir at rt for 1 hour, before being concentrated in vacuo. A mixture of sat. aq. NaHCO₃ and EtOAc (25 mL of each) was added to the crude product and the resulting suspension was stirred vigorously for 15 min. The precipitate was isolated by filtration (washing with water, then EtOAc), and dried in a vacuum oven to give ethyl 8-(3,5-dichlorophenyl)-4-hydroxy-1,7-naphthyridine-3-carboxylate. LCMS (method B): Rₜ=0.9 min, m/z=363 [M+H]⁺.

To a stirring suspension of ethyl 8-(3,5-dichlorophenyl)-4-hydroxy-1,7-naphthyridine-3-carboxylate (61 mg, 0.13 mmol) in CH₂Cl₂ (2 mL) was added oxalyl chloride (17 μL, 192 μmol) followed by DMF (1 μL, 13 μmol) and the resulting mixture was left to stir at rt for 45 min. The reaction was quenched by the addition of a sat. NaHCO₃ solution (5 mL), and the mixture was partitioned between water and CH₂Cl₂ (10 mL of each). The layers were separated. The combined organic layers were dried over anhydrous MgSO₄, filtered, and concentrated in vacuo to give ethyl 4-chloro-8-(3,5-dichlorophenyl)-1,7-naphthyridine-3-carboxylate. LCMS (method B): Rₜ=1.6 min, m/z=381 [M+H]⁺.

To ethyl 4-chloro-8-(3,5-dichlorophenyl)-1,7-naphthyridine-3-carboxylate (59 mg, 0.12 mmol) was added dimethylamine-HCl (17 mg, 0.2 mmol) in 1,4-dioxane (0.5 mL). The vial was sealed, DIPEA (73 μL, 0.41 mmol) was added and the reaction mixture was heated in the microwave at 100° C. for 30 min. The mixture was diluted with EtOAc (10 mL), washed with an aq. sat. NaHCO₃ solution (10 mL), and brine (10 mL), dried over anhydrous MgSO₄, filtered, and concentrated in vacuo to give ethyl 8-(3,5-dichlorophenyl)-4-(dimethylamino)-1,7-naphthyridine-3-carboxylate. LCMS (method B): Rₜ=1.5 min. m/z=390 [M+H]⁺.

To a stirring solution of ethyl 8-(3,5-dichlorophenyl)-4-(dimethylamino)-1,7-naphthyridine-3-carboxylate (556 mg, 1.35 mmol) in THF (14 mL) was added a solution of lithium hydroxide (99 mg, 4.05 mmol) in water (4.5 mL) and MeOH (4.5 mL). The reaction mixture was heated at 40° C. for 2 hours and left to stir at rt overnight. Then, the mixture was concentrated in vacuo, and the residue was taken up in water (25 mL). The aq. layer was washed with EtOAc (25 mL), then adjusted to pH 4 by the addition of aq. 2 M HCl, forming a suspension. The precipitate was isolated by filtration, and dried in the vacuum oven overnight to give 8-(3,5-dichlorophenyl)-4-(dimethylamino)-1,7-naphthyridine-3-carboxylic acid as a solid. LCMS (method B): Rₜ=0.78 min, m/z=362 [M+H]⁺.

To a solution of 8-(3,5-dichlorophenyl)-4-(dimethylamino)-1,7-naphthyridine-3-carboxylic acid (212 mg, 0.55 mmol) in DMF (5 mL) was added DIPEA (441 μL, 2.50 mmol). HATU (282 mg, 0.72 mmol) was added, and the reaction was left to stir for 10 min. (4S)-Chroman-4-amine-HCl (156 mg, 0.81 mmol) was added, and the reaction was left to stir at rt for 3 hour, before the mixture was concentrated in vacuo to give a residue. The residue was partitioned between EtOAc and sat. aq. NaHCO₃ (20 mL of each). The layers were separated, and the organic layer was washed with aq. 1 M HCl (20 mL), brine (20 mL), dried over anhydrous MgSO₄, filtered, and concentrated in vacuo to give a residue which was purified by column chromatography to give the title compound.

LCMS (method B): Rₜ=1.41 min, m/z=493 [M+H]⁺. ¹H-NMR (400 MHz, DMSO-d₆) δ [ppm]: 9.18 (d, J=8.4 Hz, 1H), 8.7 (s, 1H), 8.65 (d, J=5.6 Hz, 1H), 8.09 (d, J=2 Hz, 2H), 8.07 (d, J=6 Hz, 1H), 7.73 (t, J=2 Hz, 1H), 7.38 (d, J=7.6 Hz, 1 Hz), 7.18 (td, J=8.4, 1.6 Hz, 1H), 6.93 (td, J=7.6, 1.2 Hz, 1H), 6.8 (dd, J=8.4, 0.8 Hz, 1H), 5.24 (q, J=6 Hz, 1H), 4.26 (m, 2H), 3.11 (s, 6H), 2.14 (m, 2H).

The following compounds were prepared analogously by the methodology of Example 1.1:

| Ex. | Name | Structure |
|---|---|---|
| 1.2 | N-[(4S)-chroman-4-yl]-4- (dimethylamino)-8-[(1R or S)-tetralin-1-yl]-1,7- naphthyridine-3-carbox- amide | 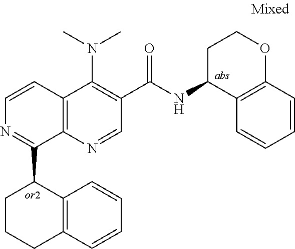 [SMILES: CN(C)c1c(C(=O)N[C@H]2CCOc3ccccc32)cnc2c([C@@H]3CCCc4ccccc43)nccc12] |
| 1.3 | N-[(4S)-chroman-4-yl]-4- (dimethylamino)-8-[(1R or S)-tetralin-1-yl]-1,7- naphthyridine-3-carbox- amide | 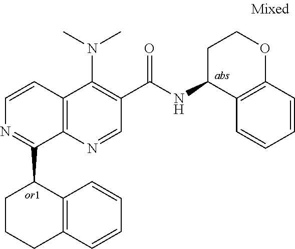 [SMILES: CN(C)c1c(C(=O)N[C@H]2CCOc3ccccc32)cnc2c([C@@H]3CCCc4ccccc43)nccc12] |
| 1.4 | N-[(4S)-chroman-4-yl]-8- (1,2-dihydronaphthalen- 1-yl)-4-(dimethylamino)- 1,7-naphthyridine-3- carboxamide | 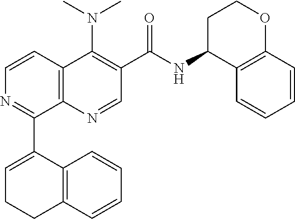 [SMILES: CN(C)c1c(C(=O)N[C@H]2CCOc3ccccc32)cnc2c(C3=CCCc4ccccc43)nccc12] |
| 1.5 | trans-8-(2,3,3a,4,5,6,7,7a- octahydroindol-1-yl)-N- [(4S)-chroman-4-yl]-4- (dimethylamino)-1,7- naphthyridine-3-carbox- amide | 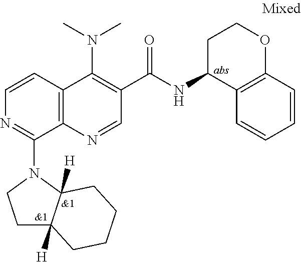 [SMILES: CN(C)c1c(C(=O)N[C@H]2CCOc3ccccc32)cnc2c(N3CC[C@H]4CCCC[C@H]43)nccc12] |
| 1.6 | cis-8-(2,3,3a,4,5,6,7,7a- octahydroindol-1-yl)-N- [(4S)-chroman-4-yl]-4- (dimethylamino)-1,7- naphthyridine-3-carbox- amide | 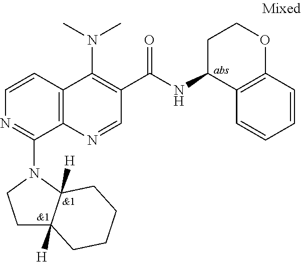 [SMILES: CN(C)c1c(C(=O)N[C@H]2CCOc3ccccc32)cnc2c(N3CC[C@H]4CCCC[C@H]43)nccc12] |
| 1.7 | 8-(5-chloroindolin-1-yl)- N-[(4S)-chroman-4-yl]- 4-(dimethylamino)-1,7- naphthyridine-3-carbox- amide | 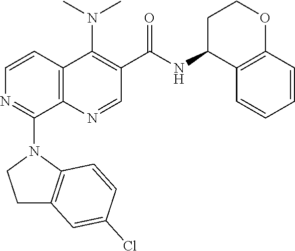 [SMILES: CN(C)c1c(C(=O)N[C@H]2CCOc3ccccc32)cnc2c(N3CCc4cc(Cl)ccc43)nccc12] |
| 1.8 | N-(5-chloroindol-1-yl)- N-[(4S)-chroman-4-yl]- 4-(dimethylamino)-1,7- naphthyridine-3-carbox- amide | 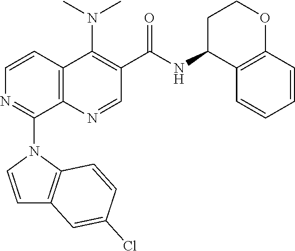 [SMILES: CN(C)c1c(C(=O)N[C@H]2CCOc3ccccc32)cnc2c(-n3ccc4cc(Cl)ccc43)nccc12] |
| 1.9 | N-[(4S)-chroman-4-yl]-8- (3,5-dichlorophenyl)-4- morpholino-1,7-naph- thyridine-3-carboxamide | 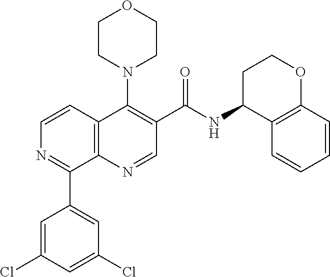 [SMILES: O=C(N[C@H]1CCOc2ccccc21)c1cnc2c(-c3cc(Cl)cc(Cl)c3)nccc2c1N1CCOCC1] |
| 1.10 | N-[(4S)-chroman-4-yl]- 4-morpholino-8-phenyl- sulfanyl-1,7-naphthyri- dine-3-carboxamide | 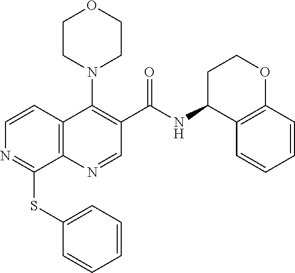 [SMILES: O=C(N[C@H]1CCOc2ccccc21)c1cnc2c(Sc3ccccc3)nccc2c1N1CCOCC1] |
| 1.11 | 8-(benzenesulfonyl)-N- [(4S)-chroman-4-yl]-4- morpholino-1,7-naphthy- ridine-3-carboxamide | 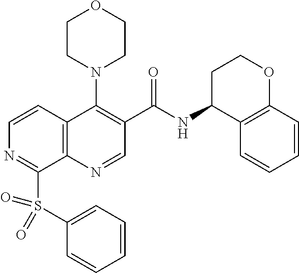 [SMILES: O=C(N[C@H]1CCOc2ccccc21)c1cnc2c(S(=O)(=O)c3ccccc3)nccc2c1N1CCOCC1] |
| 1.12 | 4-amino-N-[(4S)-chroman- 4-yl]-8-(3,5-dichlorophen- yl)-1,7-naphthyridine-3- carboxamide | 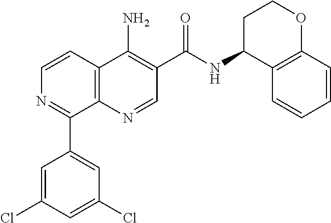 [SMILES: Nc1c(C(=O)N[C@H]2CCOc3ccccc32)cnc2c(-c3cc(Cl)cc(Cl)c3)nccc12] |
| 1.13 | N-[(4S)-chroman-4-yl]- 8-(3,5-dichlorophenyl)-4- isopropoxy-1,7-naphthyri- dine-3-carboxamide | 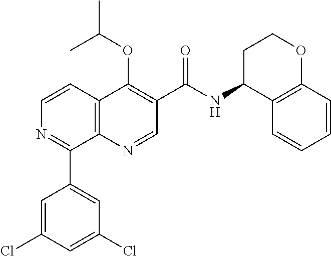 [SMILES: CC(C)Oc1c(C(=O)N[C@H]2CCOc3ccccc32)cnc2c(-c3cc(Cl)cc(Cl)c3)nccc12] |
| 1.14 | N-[(4S)-chroman-4-yl]-8- (3,5-dichlorophenyl)-4-(4- methylpiperazin-1-yl)-1,7- naphthyridine-3-carbox- amide | 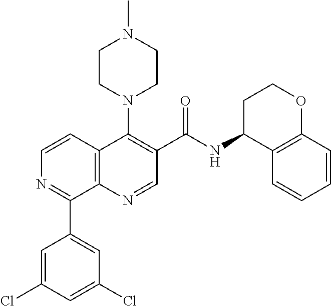 [SMILES: CN1CCN(c2c(C(=O)N[C@H]3CCOc4ccccc43)cnc3c(-c4cc(Cl)cc(Cl)c4)nccc23)CC1] |
| 1.15 | N-[(4S)-chroman-4-yl]-8- (2,6-difluorophenyl)-4-(4- methylpiperazin-1-yl)-1,7- naphthyridine-3-carbox- amide | 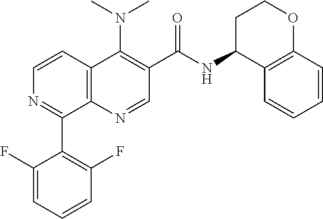 [SMILES: CN(C)c1c(C(=O)N[C@H]2CCOc3ccccc32)cnc2c(-c3c(F)cccc3F)nccc12] |
| 1.16 | N-[(4S)-chroman-4-yl]-8- (3,5-dichlorophenyl)-4- pyrrolidin-1-yl-1,7-naph- thyridine-3-carboxamide | 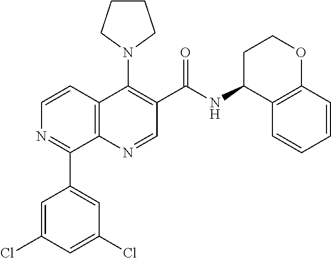 [SMILES: O=C(N[C@H]1CCOc2ccccc21)c1cnc2c(-c3cc(Cl)cc(Cl)c3)nccc2c1N1CCCC1] |
| 1.17 | 8-(3,5-dichlorophenyl)-4- (dimethylamino)-N-[(4S)- 7-fluorochroman-4-yl]- 1,7-naphthyridine-3- carboxamide | 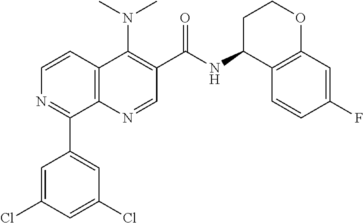 [SMILES: CN(C)c1c(C(=O)N[C@H]2CCOc3cc(F)ccc32)cnc2c(-c3cc(Cl)cc(Cl)c3)nccc12] |
| 1.18 | N-[(4S)-chroman-4-yl]-4- (dimethylamino)-8-(1H- indol-4-yl)-1,7-naphthyri- dine-3-carboxamide | 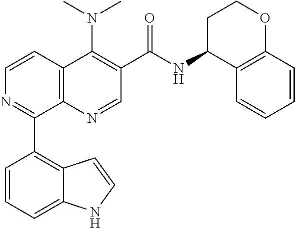 [SMILES: CN(C)c1c(C(=O)N[C@H]2CCOc3ccccc32)cnc2c(-c3cccc4[nH]ccc34)nccc12] |
| 1.19 | N-[(4S)-chroman-4-yl]-8- (3,5-dichlorophenyl)-4- phenyl-1,7-naphthyridine- 3-carboxamide | 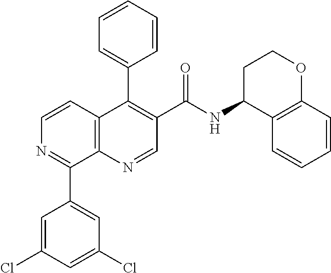 [SMILES: O=C(N[C@H]1CCOc2ccccc21)c1cnc2c(-c3cc(Cl)cc(Cl)c3)nccc2c1-c1ccccc1] |
| 1.20 | N-[(4S)-chroman-4-yl]- 4-cyclopropyl-8-(3,5- dichlorophenyl)-1,7-naph- thyridine-3-carboxamide | 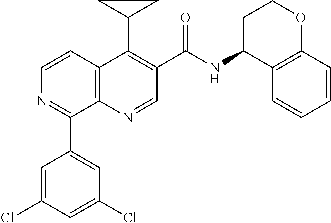 [SMILES: O=C(N[C@H]1CCOc2ccccc21)c1cnc2c(-c3cc(Cl)cc(Cl)c3)nccc2c1C1CC1] |
| 1.21 | 8-(3,5-dichlorophenyl)-4- (dimethylamino)-N-[(1S)- indan-1-yl]-1,7-naphthyri- dine-3-carboxamide | 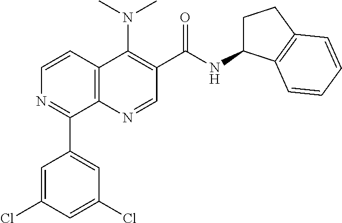 [SMILES: CN(C)c1c(C(=O)N[C@H]2CCc3ccccc32)cnc2c(-c3cc(Cl)cc(Cl)c3)nccc12] |
| 1.22 | N-[(4S)-6-cyanochroman- 4-yl]-8-(3,5-dichlorophen- yl)-4-(dimethylamino)- 1,7-naphthyridine-3- carboxamide | 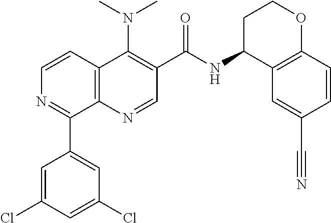 [SMILES: CN(C)c1c(C(=O)N[C@H]2CCOc3ccc(C#N)cc32)cnc2c(-c3cc(Cl)cc(Cl)c3)nccc12] |
| 1.23a | N-((4R or S)-7-chloro- chroman-4-yl)-8-(3,5- dichlorophenyl)-4- (dimethylamino)-1,7- naphthyridine-3-carbox- amide | 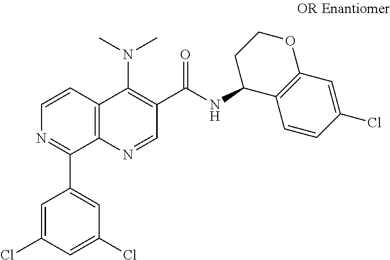 [SMILES: CN(C)c1c(C(=O)N[C@H]2CCOc3cc(Cl)ccc32)cnc2c(-c3cc(Cl)cc(Cl)c3)nccc12] |
| 1.23b | N-((4R or S)-7-chloro- chroman-4-yl)-8-(3,5- dichlorophenyl)-4- (dimethylamino)-1,7- naphthyridine-3-carbox- amide | 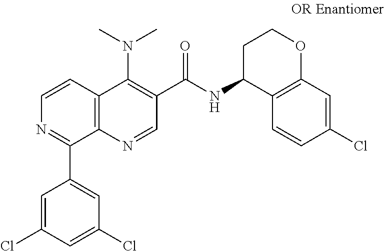 [SMILES: CN(C)c1c(C(=O)N[C@H]2CCOc3cc(Cl)ccc32)cnc2c(-c3cc(Cl)cc(Cl)c3)nccc12] |
| 1.24 | N-[(4S)-chroman-4-yl]- 8-(2,6-difluoro-3-meth- oxyphenyl)-4-(dimethyl- amino)-1,7-naphthyri- dine-3-carboxamide | 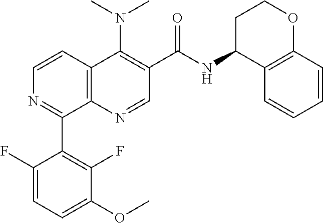 [SMILES: COc1ccc(F)c(-c2nccc3c(N(C)C)c(C(=O)N[C@H]4CCOc5ccccc54)cnc23)c1F] |
| 1.25a | 8-((4R or S)-3,5-dichloro- phenyl)-4-(dimethyl- amino)-N-(7-methoxy- chroman-4-yl)-1,7-naph- thyridine-3-carboxamide | 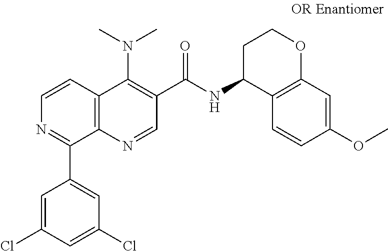 [SMILES: COc1ccc2c(c1)OCC[C@@H]2NC(=O)c1cnc2c(-c3cc(Cl)cc(Cl)c3)nccc2c1N(C)C] |
| 1.25b | 8-((4R or S)-3,5-dichloro- phenyl)-4-(dimethyl- amino)-N-(7-methoxy- chroman-4-yl)-1,7-naph- thyridine-3-carboxamide | 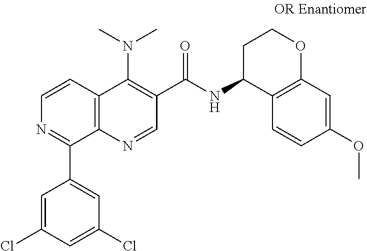 [SMILES: COc1ccc2c(c1)OCC[C@@H]2NC(=O)c1cnc2c(-c3cc(Cl)cc(Cl)c3)nccc2c1N(C)C] |
| 1.26a | 8-(3,5-dichlorophenyl)-4- (dimethylamino)-N-((4R or S)-4-methylchroman-4- yl)-1,7-naphthyridine-3- carboxamide | 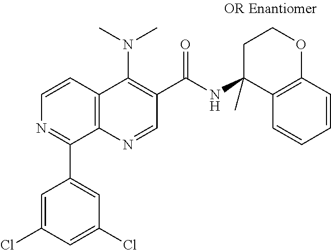 [SMILES: CN(C)c1c(C(=O)N[C@@]2(C)CCOc3ccccc32)cnc2c(-c3cc(Cl)cc(Cl)c3)nccc12] |
| 1.26b | 8-(3,5-dichlorophenyl)-4- (dimethylamino)-N-((4R or S)-4-methylchroman-4- yl)-1,7-naphthyridine-3- carboxamide | 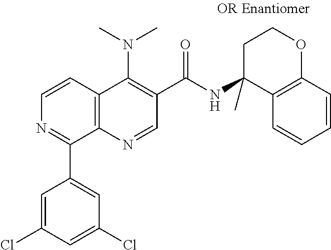 [SMILES: CN(C)c1c(C(=O)N[C@@]2(C)CCOc3ccccc32)cnc2c(-c3cc(Cl)cc(Cl)c3)nccc12] |
| 1.27a | 8-(3,5-dichlorophenyl)-4- (dimethylamino)-N-((8R or S)-5,6,7,8-tetrahydro- quinolin-5-yl)-1,7-naph- thyridine-3-carboxamide | 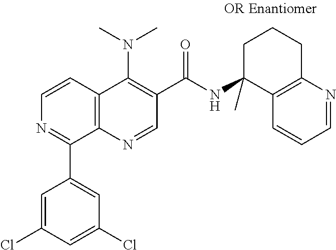 [SMILES: CN(C)c1c(C(=O)N[C@@]2(C)CCCc3ncccc32)cnc2c(-c3cc(Cl)cc(Cl)c3)nccc12] |
| 1.27b | 8-(3,5-dichlorophenyl)-4- (dimethylamino)-N-((8R or S)-5,6,7,8-tetrahydro- quinolin-5-yl)-1,7-naph- thyridine-3-carboxamide | 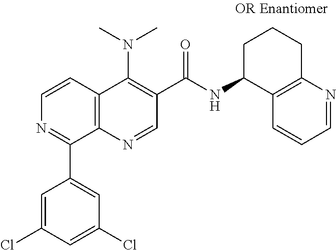 [SMILES: CN(C)c1c(C(=O)N[C@H]2CCCc3ncccc32)cnc2c(-c3cc(Cl)cc(Cl)c3)nccc12] |
| 1.28 | N-[(4S)-chroman-4-yl]-4- (dimethylamino)-8-(1- methylindolin-4-yl)-1,7- naphthyridine-3-carbox- amide | 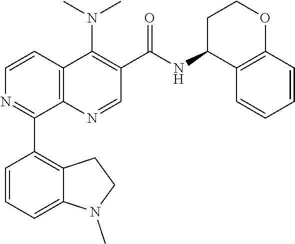 [SMILES: CN(C)c1c(C(=O)N[C@H]2CCOc3ccccc32)cnc2c(-c3cccc4c3CCN4C)nccc12] |
| 1.29 | N-[(4S)-chroman-4-yl]-8- (3,5-dichloro-2-pyridyl)- 4-(dimethylamino)-1,7- naphthyridine-3-carbox- amide | 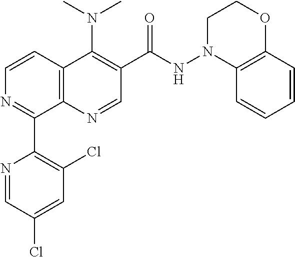 [SMILES: CN(C)c1c(C(=O)NN2CCOc3ccccc32)cnc2c(-c3ncc(Cl)cc3Cl)nccc12] |
| 1.30 | 4-chloro-N-[(4S)-chroman- 4-yl]-8-(3,5-dichlorophen- yl)-1,7-naphthyridine-3- carboxamide | 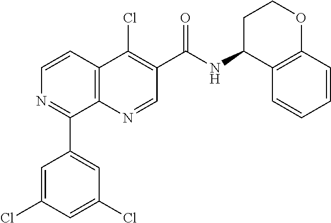 [SMILES: O=C(N[C@H]1CCOc2ccccc21)c1cnc2c(-c3cc(Cl)cc(Cl)c3)nccc2c1Cl] |
| 1.31 | N-[(4S)-chroman-4-yl]- 8-(3,5-dichlorophenyl)-4- [methoxy(methyl)amino]- 1,7-naphthyridine-3- carboxamide | 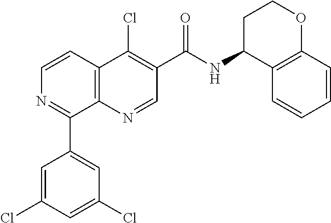 [SMILES: O=C(N[C@H]1CCOc2ccccc21)c1cnc2c(-c3cc(Cl)cc(Cl)c3)nccc2c1Cl] |
| 1.32 | 8-(3,5-dichlorophenyl)-4- (dimethylamino)-N-[(1S)- tetralin-1-yl]-1,7-naph- thyridine-3-carboxamide | 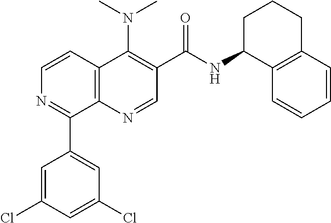 [SMILES: CN(C)c1c(C(=O)N[C@H]2CCCc3ccccc32)cnc2c(-c3cc(Cl)cc(Cl)c3)nccc12] |
| 1.33 | N-[(4S)-chroman-4-yl]-8- (3,5-dichlorophenyl)-4- methoxy-1,7-naphthyri- dine-3-carboxamide | 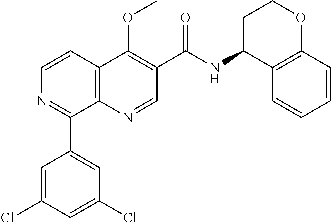 [SMILES: COc1c(C(=O)N[C@H]2CCOc3ccccc32)cnc2c(-c3cc(Cl)cc(Cl)c3)nccc12] |
| 1.34 | 8-(3,5-dichlorophenyl)-4- (dimethylamino)-N-[(1R)- indan-1-yl]-1,7-naphthyri- dine-3-carboxamide | 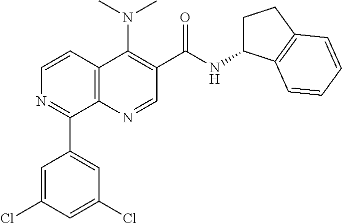 [SMILES: CN(C)c1c(C(=O)N[C@@H]2CCc3ccccc32)cnc2c(-c3cc(Cl)cc(Cl)c3)nccc12] |
| 1.35 | 8-(3,5-dichlorophenyl)-4- (dimethylamino)-N-[(1R)- tetralin-1-yl]-1,7-naph- thyridine-3-carboxamide | 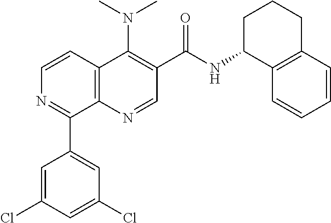 [SMILES: CN(C)c1c(C(=O)N[C@@H]2CCCc3ccccc32)cnc2c(-c3cc(Cl)cc(Cl)c3)nccc12] |
| 1.36a | 8-(3,5-dichlorophenyl)-N- ((8R or S)-6,7-dihydro- 5H-cyclopenta[b]pyridin- 5-yl)-4-(dimethylamino)- 1,7-naphthyridine-3- carboxamide | 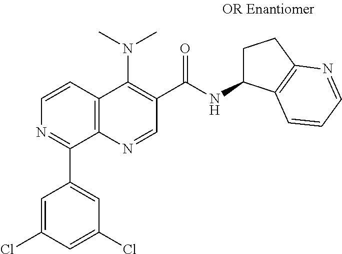 [SMILES: CN(C)c1c(C(=O)N[C@H]2CCc3ncccc32)cnc2c(-c3cc(Cl)cc(Cl)c3)nccc12] |
| 1.36b | 8-(3,5-dichlorophenyl)-N- ((8R or S)-6,7-dihydro- 5H-cyclopenta[b]pyridin- 5-yl)-4-(dimethylamino)- 1,7-naphthyridine-3- carboxamide | 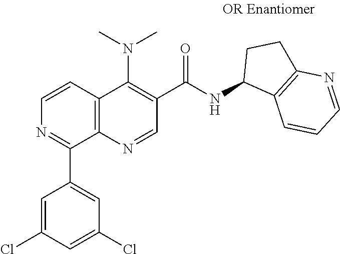 [SMILES: CN(C)c1c(C(=O)N[C@H]2CCc3ncccc32)cnc2c(-c3cc(Cl)cc(Cl)c3)nccc12] |
| 1.37 | N-[(4S)-chroman-4-yl]-8- (3,5-dichlorophenyl)-4- (4-oxoimidazolidin-1-yl)- 1,7-naphthyridine-3- carboxamide | 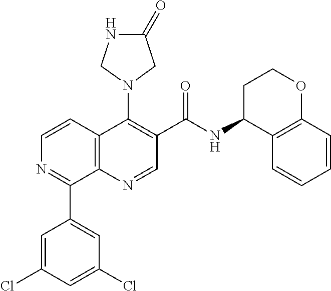 [SMILES: O=C1CN(c2c(C(=O)N[C@H]3CCOc4ccccc43)cnc3c(-c4cc(Cl)cc(Cl)c4)nccc23)CN1] |
| 1.38 | N-[(4S)-chroman-4-yl]- 4-(cyanomethyl)-8-(3,5- dichlorophenyl)-1,7-naph- thyridine-3-carboxamide | 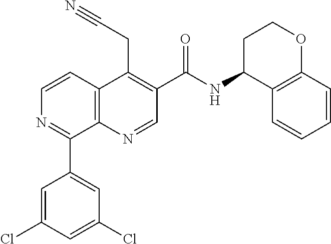 [SMILES: N#CCc1c(C(=O)N[C@H]2CCOc3ccccc32)cnc2c(-c3cc(Cl)cc(Cl)c3)nccc12] |
| 1.39 | 8-(3,5-dichlorophenyl)-4- (dimethylamino)-N-[rac- (3R,4S)-3-methylchro- man-4-yl]-1,7-naphthyri- dine-3-carboxamide | 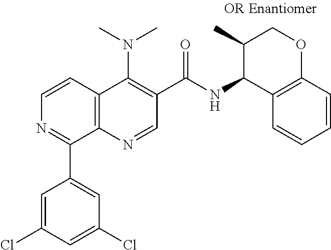 [SMILES: C[C@H]1COc2ccccc2[C@H]1NC(=O)c1cnc2c(-c3cc(Cl)cc(Cl)c3)nccc2c1N(C)C] |
| 1.40 | N-[(4S)-chroman-4-yl]- 4-cyano-8-(3,5-dichloro- phenyl)-1,7-naphthyri- dine-3-carboxamide | 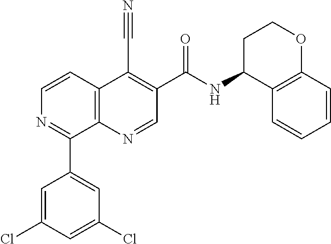 [SMILES: N#Cc1c(C(=O)N[C@H]2CCOc3ccccc32)cnc2c(-c3cc(Cl)cc(Cl)c3)nccc12] |
| 1.41 | N-[(4S)-chroman-4-yl]-8- (2,6-difluorophenoxy)-4- (dimethylamino)-1,7- naphthyridine-3-carbox- amide | 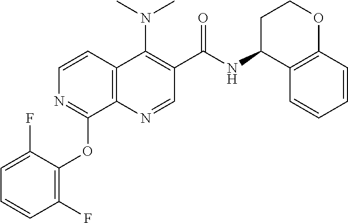 [SMILES: CN(C)c1c(C(=O)N[C@H]2CCOc3ccccc32)cnc2c(Oc3c(F)cccc3F)nccc12] |
| 1.42 | N-[(4S)-chroman-4-yl]-8- (3,5-dichlorophenyl)-4- isopropyl-1,7-naphthyri- dine-3-carboxamide | 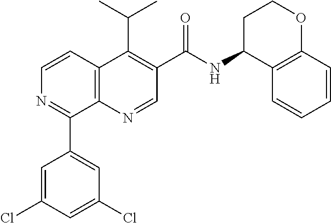 [SMILES: CC(C)c1c(C(=O)N[C@H]2CCOc3ccccc32)cnc2c(-c3cc(Cl)cc(Cl)c3)nccc12] |
| 1.43 | N-[(4S)-chroman-4-yl]-4- (dimethylamino)-8-[3- (dimethylamino)-2,6- difluorophenyl]-1,7-naph- thyridine-3-carboxamide | 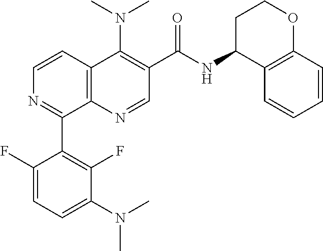 [SMILES: CN(C)c1ccc(F)c(-c2nccc3c(N(C)C)c(C(=O)N[C@H]4CCOc5ccccc54)cnc23)c1F] |
| 1.44 | N-chroman-5-yl-8-(3,5- dichlorophenyl)-4- (dimethylamino)-1,7- naphthyridine-3-carbox- amide | 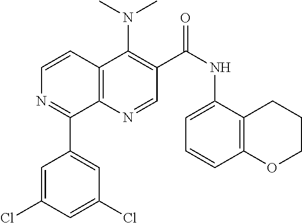 [SMILES: CN(C)c1c(C(=O)Nc2cccc3c2CCCO3)cnc2c(-c3cc(Cl)cc(Cl)c3)nccc12] |
| 1.45 | 8-(3,5-dichlorophenyl)-4- (dimethylamino)-N-[(4S)- 7-methylchroman-4-yl]- 1,7-naphthyridine-3- carboxamide | 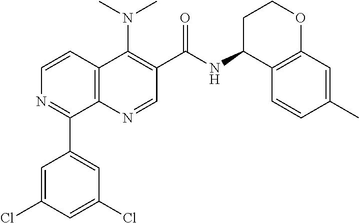 [SMILES: Cc1ccc2c(c1)OCC[C@@H]2NC(=O)c1cnc2c(-c3cc(Cl)cc(Cl)c3)nccc2c1N(C)C] |
| 1.46 | 8-(3,5-dichlorophenyl)- N-[(7S)-6,7-dihydro-5H- thieno[3,2-b]pyran-7-yl]- 4-(dimethylamino)-1,7- naphthyridine-3-carbox- amide | 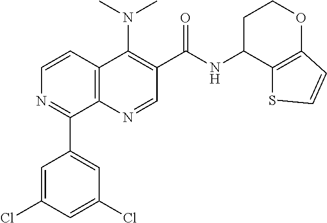 [SMILES: CN(C)c1c(C(=O)NC2CCOc3ccsc32)cnc2c(-c3cc(Cl)cc(Cl)c3)nccc12] |
| 1.47 | 8-(3,5-dichlorophenyl)-N- [(7R)-6,7-dihydro-5H- thieno[3,2-b]pyran-7-yl]- 4-(dimethylamino)-1,7- naphthyridine-3-carbox- amide | 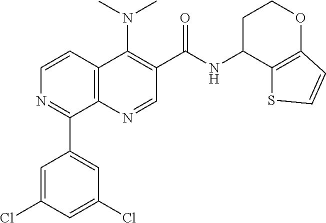 [SMILES: CN(C)c1c(C(=O)NC2CCOc3ccsc32)cnc2c(-c3cc(Cl)cc(Cl)c3)nccc12] |
| 1.48 | N-[(4S)-chroman-4-yl]-8- (3,5-difluorophenyl)-4- (dimethylamino)-1,7- naphthyridine-3-carbox- amide | 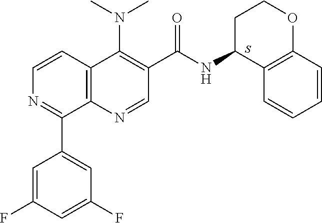 |
| 1.49 | N-[(4S)-chroman-4-yl]- 8-(2-fluorophenyl)-4- (dimethylamino)-1,7- naphthyridine-3-carbox- amide | 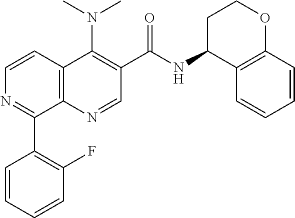 [SMILES: CN(C)c1c(C(=O)N[C@H]2CCOc3ccccc32)cnc2c(-c3ccccc3F)nccc12] |
| 1.50 | N-[(4S)-chroman-4-yl]- 4-(dimethylamino)-8- (2,3,5-trifluorophenyl)- 1,7-naphthyridine-3- carboxamide | 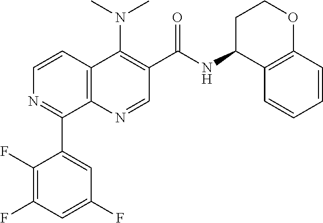 [SMILES: CN(C)c1c(C(=O)N[C@H]2CCOc3ccccc32)cnc2c(-c3cc(F)cc(F)c3F)nccc12] |
| 1.51 | N-[(4S)-chroman-4-yl]- 4-(dimethylamino)-8- (3,4,5-trifluorophenyl)- 1,7-naphthyridine-3- carboxamide | 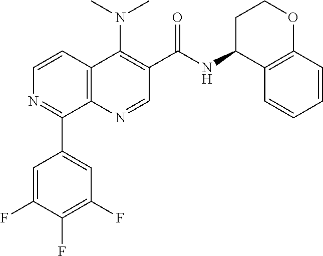 [SMILES: CN(C)c1c(C(=O)N[C@H]2CCOc3ccccc32)cnc2c(-c3cc(F)c(F)c(F)c3)nccc12] |
| 1.52 | 8-[3,5-bis(trifluorometh- yl)phenyl]-N-[(4S)-chro- man-4-yl]-4-(dimethyl- amino)-1,7-naphthyridine- 3-carboxamide | 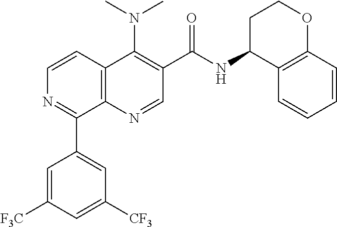 [SMILES: CN(C)c1c(C(=O)N[C@H]2CCOc3ccccc32)cnc2c(-c3cc(C(F)(F)F)cc(C(F)(F)F)c3)nccc12] |
| 1.53 | 8-(3,5-dichlorophenyl)- N-[(4S)-3,4-dihydro-2H- pyrano[3,2-c]pyridin-4- yl]-4-(dimethylamino)- 1,7-naphthyridine-3- carboxamide | 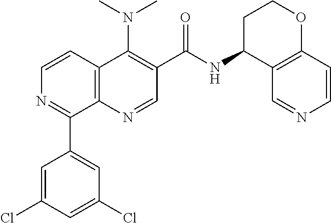 [SMILES: CN(C)c1c(C(=O)N[C@H]2CCOc3ccncc32)cnc2c(-c3cc(Cl)cc(Cl)c3)nccc12] |
| 1.54 | N-[(4S)-chroman-4-yl]- 8-(2,4-dichlorophenyl)- 4-(dimethylamino)-1,7- naphthyridine-3-carbox- amide | 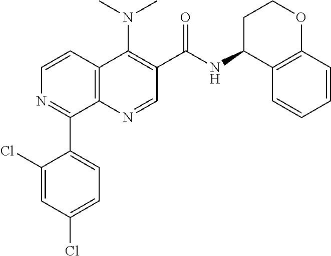 [SMILES: CN(C)c1c(C(=O)N[C@H]2CCOc3ccccc32)cnc2c(-c3ccc(Cl)cc3Cl)nccc12] |
| 1.55 | N-[(4S)-chroman-4-yl]- 8-(2,3-dichlorophenyl)- 4-(dimethylamino)-1,7- naphthyridine-3-carbox- amide | 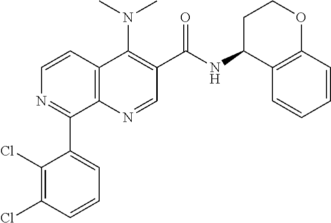 [SMILES: CN(C)c1c(C(=O)N[C@H]2CCOc3ccccc32)cnc2c(-c3cccc(Cl)c3Cl)nccc12] |

Examples 1.2 and 1.3 were separated by SFC: The separation was performed on Chiralpak® AD-H with column dimensions of 250 mm×30 mm (5 m), a flow rate of 90 g/min, and a CO₂-based mobile phase with 35% iPrOH containing 0.2% N,N-dimethylethylamine as additive.

### Example 2.1

### N-[(4S)-chroman-4-yl]-8-(3,5-dichlorophenyl)-4-(dimethylamino) isoquinoline-3-carboxamide

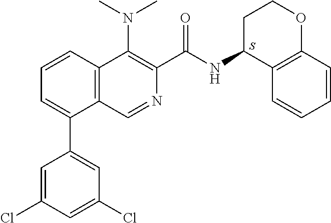

A mixture of 8-bromoisoquinoline (0.5 g, 2.43 mmol), (3,5-dichlorophenyl) boronic acid (0.46 g, 2.41 mmol), K₂CO₃ (2.8 g, 20.4 mmol) and tetrakis(triphenylphosphine) palladium(0) (0.15 g, 0.12 mmol) was placed under N₂-atmosphere and treated with 1,2-dimethoxyethane (8.69 g, 96.4 mmol) and water (10 mL). The reaction vessel was evacuated and back-filled with N₂ three times. The reaction mixture was then heated at 90° C. for 2 hours. The mixture was allowed to cool to rt, before being diluted with water (20 mL) and extracted with CH₂Cl₂ (2×20 mL). The combined organic layers were dried over anhydrous MgSO₄, filtered, concentrated in vacuo to give a residue which was purified by column chromatography to give 8-(3,5-dichlorophenyl) isoquinoline. LCMS (method B): Rₜ=1.36 min, m/z=273.8 [M+H]⁺. A mixture of 8-(3,5-dichlorophenyl)-isoquinoline (0.56 g, 1.95 mmol) and N-bromosuccinimide (0.54 g, 2.94 mmol) was placed under N₂-atmosphere and treated with acetic acid (9 mL, 157.1 mmol). The resulting mixture was warmed to 60° C. and was allowed to stir for 2 hours. The reaction mixture was allowed to cool down to rt, before being poured onto water (60 mL) and extracted with CH₂Cl₂ (3×30 mL). The combined organic layers were dried over anhydrous MgSO₄, filtered, concentrated in vacuo to give a residue which was purified by column chromatography to afford 4-bromo-8-(3,5-dichlorophenyl)-isoquinoline. LCMS (method B): Rₜ=1.57 min, m/z=351.8 [M+H]⁺. A mixture of 4-bromo-8-(3,5-dichlorophenyl) isoquinoline (0.46 g, 1.246 mmol), Cs₂CO₃ (0.82 g, 2.53 mmol), tert-butyl carbamate (0.44 g, 3.8 mmol) and [(4,5-bis(diphenylphosphino)-9,9-dimethylxanthene)-2-(2′-amino-1,1′-biphenyl)]palladium(II) methanesulfonate (Xantphos Pd G3, 70 mg, 74 μmol) was placed under N₂-atmosphere and treated with 1,4-dioxane (6.5 mL). The reaction vessel was evacuated and back-filled with N₂ three times. The resulting mixture was heated at 100° C. for 5 hours. Then, the mixture was allowed to cool down to rt, before being diluted with sat. aq. NaHCO₃ (20 mL) and extracted with CH₂Cl₂ (3×25 mL). The combined organic layers were dried over anhydrous MgSO₄, filtered and concentrated in vacuo to give a residue which was purified by column chromatography to give tert-butyl N-[8-(3,5-dichlorophenyl)-4-isoquinolyl]carbamate. LCMS (method B): Rₜ=1.42 min, m/z=389 [M+H]⁺. A suspension of tert-butyl N-[8-(3, 5-dichlorophenyl)-4-isoquinolyl]carbamate (0.48 g, 0.93 mmol) in CH₂Cl₂ (5 mL) was placed under N₂-atmosphere and was treated with TFA (2 mL, 26.45 mmol). The resulting solution was allowed to stir at rt for 2 hours. The reaction mixture was then concentrated in vacuo. The residues were diluted with sat. aq. NaHCO₃ (20 mL) and extracted with EtOAc (3×20 mL). The combined organic layers were washed with brine (20 mL), dried over anhydrous MgSO₄, filtered, and concentrated in vacuo to give 8-(3,5-dichlorophenyl)isoquinolin-4-amine. LCMS (method B): Rₜ=1.17 min, m/z=289 [M+H]⁺.

A solution of 8-(3,5-dichlorophenyl)isoquinolin-4-amine (0.2 g, 0.65 mmol) in DMF (5 mL) was placed under N₂-atmosphere, cooled to 0° C. in an ice bath and treated with N-bromosuccinimide (0.12 g, 0.65 mmol). The resulting mixture was stirred for 30 min. at 0° C. Then, the mixture was poured onto water (30 mL) and was extracted with EtOAc (3×25 mL). The combined organic layers were washed with brine (30 mL), dried over anhydrous MgSO₄, filtered, and concentrated in vacuo. The residue was purified by column chromatography to give 3-bromo-8-(3,5-dichlorophenyl) isoquinolin-4-amine. LCMS (method B): Rₜ=1.4 min, m/z=366.8 [M+H]⁺.

A suspension of 3-bromo-8-(3,5-dichlorophenyl)isoquinolin-4-amine (0.15 g, 0.4 mmol) in formic acid (1.2 mL, 31 mmol) was placed under N₂-atmosphere, before being treated with a formaldehyde solution (37 wt. % in water, 1.8 mL, 24.2 mmol). The resulting suspension was heated at 100° C. for 3 hours. The reaction mixture then was allowed to cool down to rt, before being carefully poured onto sat. aq. NaHCO₃ (25 mL) and extracted with CH₂Cl₂ (3×15 mL). The combined organic layers were evaporated to give a residue which was purified by column chromatography to give 3-bromo-8-(3,5-dichlorophenyl)-N,N-dimethyl-isoquinolin-4-amine. LCMS (method B): Rₜ=1.72 min, m/z=394.8 [M+H]⁺.

A suspension of (4S)-chroman-4-amine·HCl (67 mg, 0.36 mmol) and 3-bromo-8-(3,5-dichlorophenyl)-N,N-dimethyl-isoquinolin-4-amine (0.12 g, 0.29 mmol) in 1,4-dioxane (10 mL) was treated with NEt₃ (0.08 mL, 0.57 mmol) and [(4,5-bis(diphenylphosphino)-9,9-dimethylxanthene)-2-(2′-amino-1,1′-biphenyl)]palladium(II) methanesulfonate (Xantphos Pd G3, 17 mg, 170 μmol), before being allowed to stir at 70° C. under an CO-atmosphere (275 kPa) overnight. The reaction mixture was diluted with water (20 mL) and extracted with EtOAc (3×20 mL). The combined organic layers were washed with brine (30 mL), dried over anhydrous MgSO₄, filtered, and concentrated in vacuo to give a residue. The residue was purified by column chromatography to give the title compound. LCMS (method B): Rₜ=1.60 min, m/z=492.0 [M+H]⁺. ¹H-NMR (400 MHz, DMSO-d6) δ[ppm]: 8.90 (d, J=8.4 Hz, 1H), 8.80 (s, 1H), 8.36 (d, J=8.6 Hz, 1H), 7.90 (dd, J=8.6, 7.2 Hz, 1H), 7.77 (t, J=2 Hz, 1H), 7.68 (d, J=7 Hz, 1H), 7.62 (d, J=2 Hz, 2H), 7.31 (d, J=7.6 Hz, 1H), 7.16 (m, 1H), 6.91 (m, 1H), 6.87 (dd, J=8.2, 0.8 Hz, 1H), 5.31 (m, 1H), 4.27 (m, 2H), 2.976 (s, 6H), 2.17 (m, 1H), 2.07 (m, 1H).

### Example 3.1

### N-[(4S)-chroman-4-yl]-8-(3,5-dichlorophenyl)-4-(dimethylamino)-2,7-naphthyridine-3-carboxamide

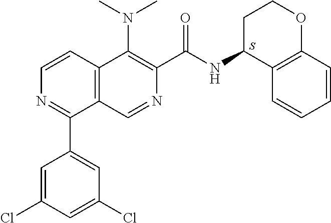

A suspension of NaH (60% in mineral oil, 1.76 g, 44 mmol) in THF (26 mL) was placed under N₂-atmosphere, cooled to 0° C. in an ice bath and treated slowly with diethyl malonate (6.4 mL, 42 mmol). The resulting mixture was allowed to stir at 0° C. for 15 min., before it was treated portionwise with 3-bromo-4-chloro-5-nitro-pyridine (5.01 g, 21.1 mmol). The ice bath was removed and the resulting reaction mixture was allowed to stir for 30 min. at rt. Then, the mixture was poured onto ice cooled aq. NH₄Cl (80 mL) and extracted with EtOAc (3×60 mL). The combined organic layers were washed with brine (60 mL), dried over anhydrous MgSO₄, filtered, and concentrated in vacuo to give diethyl 2-(3-bromo-5-nitro-4-pyridyl)propanedioate (9.53 g, 21.1 mmol) which was then treated with HCl (5 M) in water (60 mL). The resulting mixture was heated at reflux overnight. The reaction mixture was allowed to cool to rt, before being cooled over ice and basified by the dropwise addition of a 50% NaOH solution (15 mL). The resulting aq. layer was extracted with EtOAc (3×60 mL). The combined organic layers were washed with brine (80 mL) and concentrated in vacuo to give a residue which was purified by column chromatography to give 3-bromo-4-methyl-5-nitro-pyridine. LCMS (method B): Rₜ=0.93 min, no significant mass ion observed.

A mixture of 3-bromo-4-methyl-5-nitro-pyridine (4.39 g, 20.2 mmol), Cyanation Kit J (J. Org. Chem. 2018, 83, 4922-4931) (5.03 g, 20.4 mmol) and KOAc (1 g, 10.16 mmol) was placed under N₂-atmosphere, mixed with 1,4-dioxane (20 mL) and de-gassed water (20 mL). The reaction vessel was evacuated and back-filled with N₂ three times. The mixture was then heated at 90° C. for 3 hours. The reaction mixture was allowed to cool down to rt before being poured onto water (150 mL) and extracted with EtOAc (3×70 mL). The combined organic layers were washed with brine (80 mL), dried over anhydrous MgSO₄, filtered, and concentrated in vacuo. The resulting residue was purified by column chromatography to give 4-methyl-5-nitro-pyridine-3-carbonitrile. LCMS (method B): Rₜ=0.94 min, m/z=no significant mass ion observed.

To a solution of 4-methyl-5-nitro-pyridine-3-carbonitrile (49 mg, 0.27 mmol) in CH₂Cl₂ (0.7 mL) under N₂-atmosphere was added DMF-DMA (75 μL, 0.56 mmol) and the resulting mixture was stirred for 2 hours at 40° C. The reaction mixture was allowed to cool down to rt and was then concentrated in vacuo to give 4-[(E)-2-(dimethylamino) vinyl]-5-nitro-pyridine-3-carbonitrile (63 mg, 0.27 mmol) which was treated with acetic acid (0.75 mL 13 mmol) and HBr (5.02 mol/L) in acetic acid (1.10 mL, 5.52 mmol) under N₂-atmosphere, and the mixture was then heated at 60° C. for 45 min. After this time, the mixture was slowly added to a stirring aq. solution of NaHCO₃ (40 mL) and then extracted with EtOAc (3×15 mL). The combined organic layers were evaporated to give a residue which was purified by column chromatography to give 1-bromo-5-nitro-2,7-naphthyridine. LCMS (method B): Rₜ=0.51 min, m/z=192.0 [M+H]⁺.

A mixture of 5-nitro-2H-2,7-naphthyridin-1-one (0.23 g, 1.18 mmol) and POBr₃ (1.71 g, 5.96 mmol) in a microwave vial was placed under N₂-atmosphere and diluted with CH₃CN (4 mL). The resulting mixture was irradiated under microwave radiation at 130° C. for 1 hour. The reaction mixture was then poured onto water (20 mL) and extracted with CH₂Cl₂ (3×20 mL). The combined organic layers were dried over anhydrous MgSO₄, filtered and concentrated in vacuo. The resulting residue was purified by column chromatography to give 1-bromo-5-nitro-2,7-naphthyridine. LCMS (method B): Rₜ=0.82 min, m/z=no significant mass ion observed.

A sealed microwave vial containing a mixture of 1-bromo-5-nitro-2,7-naphthyridine (48 mg, 0.18 mmol), (3,5-dichlorophenyl) boronic acid (46 mg, 0.24 mmol), chloro(crotyl)(tri-t-butylphosphine) Pd (II)) (5.5 mg, 13 μmol) and KF (35 mg, 0.6 mmol) was evacuated and back filled with N₂. The mixture was mixed with THF (0.60 mL) and was then irradiated under microwave radiation at 100° C. for 90 min. The mixture was filtered through Celite® (washed through with CH₂Cl₂), diluted with water (30 mL) and extracted with CH₂Cl₂ (4×20 mL). The combined organic layers were dried over anhydrous MgSO₄, filtered, and concentrated in vacuo. The residue was purified by column chromatography to give 1-(3,5-dichlorophenyl)-5-nitro-2,7-naphthyridine. LCMS (method B): Rₜ=1.29 min, m/z=319.8 [M+H]⁺.

A mixture of 1-(3,5-dichlorophenyl)-5-nitro-2,7-naphthyridine (0.24 g, 0.46 mmol), NH₄Cl (84 mg, 1.565 mmol, and iron (85 mg, 1.49 mmol) was placed under N₂-atmosphere and mixed with THF (2.5 mL), EtOH (2.5 mL) and water (1.2 mL). The reaction mixture was heated at 75° C. and was stirred for 20 min. Then, the mixture was allowed to cool to rt, before being filtered through Celite®. The filtrate was concentrated in vacuo to give a residue which was purified by column chromatography and afforded 8-(3,5-dichlorophenyl)-2,7-naphthyridin-4-amine. LCMS (method B): Rₜ=1.01 min, m/z=290.0 [M+H]⁺.

Under N₂-atmosphere in a salt/ice bath, a solution of 8-(3,5-dichlorophenyl)-2,7-naphthyridin-4-amine (0.1 g, 0.32 mmol) in DMF (2 mL) was treated with N-bromosuccinimide (57 mg, 0.32 mmol) and was then allowed to stir for 75 min. After this time, the mixture was poured onto water (25 mL) and extracted with CH₂Cl₂ (3×20 mL). The combined organic layers were dried over anhydrous MgSO₄, filtered, and concentrated in vacuo. The resulting residue was purified by column chromatography to give 3-bromo-8-(3,5-dichlorophenyl)-2,7-naphthyridin-4-amine. LCMS (method B): Rₜ=1.25 min, m/z=367.8 [M+H]⁺.

3-Bromo-8-(3,5-dichlorophenyl)-2,7-naphthyridin-4-amine (85 mg, 0.22 mmol) was treated with formic acid (0.6 mL, 15.9 mmol) and formaldehyde (37 wt. % solution in water, 1 mL, 13.4 mmol) under N₂-atmosphere. The resulting suspension was heated at 120° C. for 5 hours. Then, the mixture was allowed to cool down to rt, before being poured onto sat. aq. NaHCO₃ (20 mL) and extracted with CH₂Cl₂ (4×10 mL). The combined organic layers were dried over anhydrous MgSO₄, filtered, and concentrated in vacuo to give a residue which was purified by column chromatography and afforded 3-bromo-8-(3,5-dichlorophenyl)-N,N-dimethyl-2,7-naphthyridin-4-amine. LCMS (method B): Rₜ=1.56 min, m/z=396 [M+H]⁺.

A mixture of (4S)-chroman-4-amine-HCl (37 mg, 0.2 mmol), 3-bromo-8-(3,5-dichlorophenyl)-N,N-dimethyl-2,7-naphthyridin-4-amine (67 mg, 0.16 mmol) and NEt₃ (50 μL, 0.36 mmol) in 1,4-dioxane (6 mL) was treated with [(4,5-bis(diphenylphosphino)-9,9-dimethylxanthene)-2-(2′-amino-1,1′-biphenyl)]palladium(II) methanesulfonate (Xantphos Pd G3, 8 mg, 8 μmol). The reaction mixture was then allowed to stir at 100° C. under an atmosphere of CO-atmosphere (380 kPa) overnight. The reaction mixture was allowed to cool to rt. The reaction mixture was concentrated in vacuo. The residues were subjected to column chromatography. LCMS (method B): Rₜ=1.47 min, m/z=493 [M+H]⁺. ¹H-NMR (400 MHz, DMSO-d6) δ[ppm]: 9.03 (d, J=8.4 Hz, 1H), 9.02 (s, 1H), 8.85 (d, J=5.9 Hz, 1H), 8.15 (d, J=5.6 Hz, 1H), 7.86 (t, J=2 Hz, 1H), 7.80 (d, J=1.8 Hz, 2H), 7.31 (d, J=7.2 Hz, 1H), 7.17 (td, J=7.2, 1.2 Hz, 1H), 6.92 (td, J=7.4, 1 Hz, 1H), 6.79 (d, J=7.4 Hz, 1H), 5.30 (m, 1H), 4.273 (t, J=5.3 Hz, 2H), 2.98 (s, 6H), 2.18 (m, 1H), 2.07 (m, 1H).

### Example 4.1

### N-[(4S)-chroman-4-yl]-1-(3,5-dichlorophenyl)-5-(dimethylamino)isoquinoline-6-carboxamide

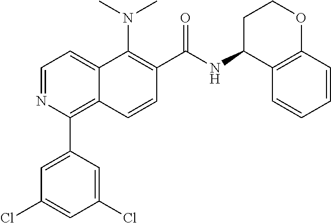 [SMILES: CN(C)c1c(C(=O)N[C@H]2CCOc3ccccc32)ccc2c(-c3cc(Cl)cc(Cl)c3)nccc12]

1-Chloroisoquinoline (1.08 g, 5.94 mmol) in a microwave vial was treated with sulfuric acid (17.8 mol/L) in water (15 mL) and nitric acid (0.1 mol/L) in water (0.65 mL) before being allowed to stir for 20 min. at rt. The reaction mixture was slowly added to a stirring sat. aq. NaHCO₃ solution. The aq. phase was extracted with CH₂Cl₂ (3×30 mL). The combined organic layers were filtered and concentrated in vacuo to give 1-chloro-5-nitro-isoquinoline. LCMS (method B): Rₜ=1.01 min, m/z=208.8 [M+H]⁺.

A sealed microwave vial containing a mixture of 1-chloro-5-nitro-isoquinoline (0.7 g, 3.02 mmol), (3,5-dichlorophenyl)boronic acid (0.53 g, 2.72 mmol), Na₂CO₃ (0.64 g, 6.05 mmol) and 1,1′-bis(diphenylphosphino) ferrocene-Pd(II)·CH₂Cl₂ complex (0.12 g, 0.15 mmol) was evacuated and back filled with N₂. The mixture was treated with 1,4-dioxane (13.5 mL) and de-gassed water (5 mL) before being irradiated under microwave radiation to 80° C. for 30 min. The reaction mixture was treated with CH₂Cl₂ (40 mL) and water (40 mL) and the two layers were separated. The aq. phase was extracted with further CH₂Cl₂ (3×15 mL) and the combined organic layers were filtered and concentrated in vacuo. The residues were subjected to column chromatography (eluting with 0-40% tert-butylmethyl ether in cyclohexane) to give 1-(3,5-dichlorophenyl)-5-nitro-isoquinoline. LCMS (method B): Rₜ=1.40 min., m/z=319.0 [M+H]⁺.

A mixture of 1-(3,5-dichlorophenyl)-5-nitro-isoquinoline (0.68 g, 2.02 mmol), NH₄Cl (0.33 g, 6.17 mmol) and iron (0.361 g, 6.33 mmol) in a flask was placed under N₂-atmosphere and dissolved with THF (10 mL), EtOH (10 mL) and water (5 mL). The resulting suspension was warmed to 75° C. and was allowed to stir for 20 min. The reaction mixture was allowed to cool to rt before being filtered through Celite© and concentrated in vacuo. The residues were treated with 5% MeOH in CH₂Cl₂ and the formed precipitate was filtered and dried under vacuum to give 1-(3,5-dichlorophenyl)isoquinolin-5-amine. LCMS (method B): Rₜ=1.24 min., m/z=289.0 [M+H]⁺.

A suspension of 1-(3,5-dichlorophenyl)isoquinolin-5-amine (0.45 g, 1.43 mmol) in DMF (6 mL) in a flask was placed under N₂-atmosphere, cooled over an ice/brine bath and treated with N-bromosuccinimide (0.26 g, 1.44 mmol). The resulting dark brown solution was allowed to stir at −10° C. for 1 hour. Water and CH₂Cl₂ were added (10 mL each). The two phases were extracted with further CH₂Cl₂ (3×10 mL). The combined organic phases were filtered and concentrated in vacuo. The residues were subjected to column chromatography (0-50% EtOAc in cyclohexane) to give 6-bromo-1-(3,5-dichlorophenyl)isoquinolin-5-amine. LCMS (method B): Rₜ=1.44 min., m/z=366.8 [M+H]⁺.

A solution of 6-bromo-1-(3,5-dichlorophenyl)isoquinolin-5-amine (0.39 g, 0.96 mmol) in formic acid (2.3 mL, 61 mmol) in a flask was placed under N₂-atmosphere and treated with formaldehyde solution (37 wt. % in water) (4.7 g, 58 mmol, 4.3 mL). The resulting mixture was warmed to 100° C. and was allowed to stir for 2.5 hours. The reaction mixture was allowed to cool to rt before being quenched with sat. aq. NaHCO₃ (30 mL) and extracted with CH₂Cl₂ (3×0 mL). The combined organic layers were filtered and concentrated in vacuo. The residues were subjected to column chromatography (0-20% tert-butylmethyl ether in cyclohexane) to give 6-bromo-1-(3,5-dichlorophenyl)-N,N-dimethyl-isoquinolin-5-amine. LCMS (method B): Rₜ=1.73 min., m/z=395.0 [M+H]⁺.

A solution of 6-bromo-1-(3,5-dichlorophenyl)-N,N-dimethyl-isoquinolin-5-amine (0.36 g, 0.81 mmol), (4S)-chroman-4-amine-HCl (0.18 g, 0.99 mmol) and NEt₃ (0.23 mL 1.65 mmol) in 1,4-dioxane (11 mL) in a flask was treated with [(4,5-bis(diphenylphosphino)-9,9-dimethylxanthene)-2-(2′-amino-1,1′-biphenyl)]palladium(II) methanesulfonate (Xantphos Pd G3, (0.048 g, 0.048 mmol) before being allowed to stir under CO-atmosphere (379 kPa) at 100° C. overnight. The reaction mixture was diluted with water (20 mL) and extracted with EtOAc (3×20 mL). The combined organic layers were washed with brine (30 mL), dried over anhydrous MgSO₄, filtered, and concentrated in vacuo. The resulting residue was purified by column chromatography to give the title compound. LCMS (method B): Rₜ=1.50 min., m/z=492.0 [M+H]⁺. ¹H-NMR (400 MHz, DMSO-d₆) δ [ppm]: 9.10 (d, J=8.20 Hz, 1H), 8.63 (d, J=5.9 Hz, 1H), 8.12 (d, J=5.9 Hz, 1H), 7.81 (t, J=2.0 Hz, 1H), 7.69-7.67 (m, 3H), 7.54 (d, J=8.6 Hz, 1H), 7.34 (d, J=7.2 Hz, 1H), 7.17 (td, J=8.4, 1.2 Hz, 1H), 6.93 (td, J=7.4, 1 Hz, 1H), 6.81-6.79 (m, 1H), 5.29-5.25 (m, 1H), 4.30-4.23 (m, 2H), 2.97 (s, 6H), 2.24-2.18 (m, 2H).

### Example 5.1

### (4S)-chroman-4-yl]-5-(3,5-dichlorophenyl)-1-(dimethylamino)naphthalene-2-carboxamide

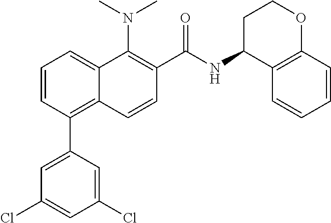 [SMILES: CN(C)c1c(C(=O)N[C@H]2CCOc3ccccc32)ccc2c(-c3cc(Cl)cc(Cl)c3)cccc12]

A mixture of 1-bromo-5-nitro-naphthalene (1.04 g, 4.13 mmol), (3,5-dichlorophenyl)-boronic acid (0.7 g, 3.60 mmol), Na₂CO₃ (0.862 g, 8.10 mmol) and 1,1′-bis(diphenylphosphino) ferrocene-Pd(II)—CH₂Cl₂ complex (0.156 g, 0.20 mmol) was de-gassed and re-filled with N₂ three times. The mixture was treated with 1,4-dioxane (20 mL) and de-gassed water (6 mL), warmed to 80° C. and stirred for 45 min. The reaction mixture was allowed to cool to rt before being diluted with water (40 mL) and extracted with CH₂Cl₂ (3×30 mL). The combined organic layers were dried over anhydrous Na₂SO₄, filtered and concentrated in vacuo. The residues were purified by column chromatography (0-25% tert-butylmethyl ether in cyclohexane) to give 1-(3,5-dichlorophenyl)-5-nitro-naphthalene. LCMS (method A): Rₜ=1.61 min, no significant mass ion observed.

A mixture of 1-(3,5-dichlorophenyl)-5-nitro-naphthalene (0.928 g, 2.77 mmol), NH₄Cl (0.468 g, 8.72 mmol) and iron (0.47 g, 8.28 mmol) was placed under N₂-atmosphere before adding THF (14 mL), EtOH (14 mL) and water (7 mL). The resulting mixture was warmed to 75° C. and was allowed to stir for 45 min, forming a black mixture. The reaction mixture was allowed to cool to rt before being filtered through Celite® (washed through with CH₂Cl₂). The filtrate was concentrated in vacuo, treated with sat. aq. NaHCO₃ (50 mL) and extracted with CH₂Cl₂ (3×25 mL). The combined organic layers were filtered through Celite® and concentrated in vacuo to give 5-(3,5-dichlorophenyl)naphthalen-1-amine. LCMS (method B): Rₜ=1.49 min., m/z=288.0 [M+H]⁺.

A solution of 5-(3,5-dichlorophenyl)naphthalen-1-amine (0.88 g, 2.60 mmol) in DMF (10 mL) was placed N₂-atmosphere, cooled over an ice/salt bath to approximately −5° C. and treated with N-bromosuccinimide (0.47 g, 2.58 mmol). The reaction mixture was allowed to stir for 30 min. The reaction mixture was treated with sat. aq. NaHCO₃-solution (50 mL). The mixture was extracted with CH₂Cl₂ (3×30 mL) and the combined organic phases were concentrated in vacuo. The residues were purified by column chromatography (0-30% EtOAc in cyclohexane) to give 2-bromo-5-(3,5-dichlorophenyl)naphthalen-1-amine. LCMS (method B): Rₜ=1.64 min, m/z=365.8 [M+H]⁺.

A suspension of 2-bromo-5-(3,5-dichlorophenyl)naphthalen-1-amine (0.73 g, 1.79 mmol) in formic acid (6 mL, 160 mmol) in a 100 mL round bottomed flask was placed under N2-atmosphere and treated with a formaldehyde solution (37 wt. % in water) (8 mL, 110 mmol). The resulting suspension was warmed to 100° C. and was allowed to stir for 1 hour. The reaction mixture was allowed to cool to rt before adding sat. aq. NaHCO₃ (60 mL). The mixture was then extracted with CH₂Cl₂ (3×20 mL) and the combined organic phases were dried over anhydrous Na₂SO₄, filtered and concentrated in vacuo. The residues were purified by column chromatography (0-5% tert-butyl methyl ether in cyclohexane) to give 2-bromo-5-(3,5-dichlorophenyl)-N,N-dimethyl-naphthalen-1-amine. LCMS (method B): Rₜ=1.93 min, m/z=393.8 [M+H]⁺.

A solution of 2-bromo-5-(3,5-dichlorophenyl)-N,N-dimethyl-naphthalen-1-amine (532 mg, 1.279 mmol) in 1,4-dioxane (10 mL) was treated with MeOH (10 mL), NEt₃ (0.54 mL, 3.9 mmol) and 1,1′-bis(diphenylphosphino) ferrocene —Pd(II)—CH₂C₁₂ complex (0.103 g, 0.134 mmol) before being stirred at 100° C. under CO-atmosphere (0.036 g, 1.28 mmol, 50 psi) for 16 hours. The reaction mixture was allowed to cool to r. The reaction mixture was filtered and concentrated in vacuo. The residue was purified by column chromatography (0-10% tert-butylmethyl ether in cyclohexane) to give methyl 5-(3,5-dichlorophenyl)-1-(dimethylamino)naphthalene-2-carboxylate. LCMS (method B): Rₜ=1.75 min, m/z=374.0 [M+H]⁺.

A solution of methyl 5-(3,5-dichlorophenyl)-1-(dimethylamino)naphthalene-2-carboxylate (0.42 g, 1.01 mmol) in THF (10 mL) was treated with 1,4-dioxane (15 mL), water (5 mL) and lithium hydroxide (0.26 g, 10.23 mmol). The reaction mixture was warmed to 85° C. and was allowed to stir for 2 hours. The reaction mixture was cooled to rt before being treated with 2 M HCl (until pH 8). The aq. phase was extracted with CH₂Cl₂ (3×25 mL). The combined organic layers were filtered, concentrated in vacuo and dried to give 5-(3,5-dichlorophenyl)-1-(dimethylamino)naphthalene-2-carboxylic acid. LCMS (method B): Rₜ=1.10 min, m/z=358.0 [M−H]⁻.

A mixture of 5-(3,5-dichlorophenyl)-1-(dimethylamino)naphthalene-2-carboxylic acid (0.225 g, 0.5 mmol), (4S)-chroman-4-amine-HCl (0.11 g, 0.58 mmol) and PyBOP (0.414 g, 0.78 mmol) in a 50 mL round bottomed flask was placed under N₂-atmosphere and treated with THF (5 mL) and NEt₃ (1.8 mmol, 0.25 mL). The resulting reaction mixture was stirred for 1 hour at rt. The reaction mixture was poured onto sat. aq. NaHCO₃ (20 mL) and extracted with CH₂Cl₂ (3×15 mL). The combined organic layers were dried over anhydrous Na₂SO₄, filtered and concentrated in vacuo. The residues were purified by column chromatography (0-15% EtOAc in cyclohexane) to give the title compound. LCMS (method B): Rₜ=1.63 min, m/z=491.0 [M+H]⁺. ¹H-NMR (400 MHz, DMSO-d6) δ[ppm]9.03 (d, J=8.4 Hz, 1H), 8.33 (d, J=8 Hz, 1H), 7.77 (t, J=2 Hz, 1H), 7.64 (d, J=7.2 Hz, 1H), 7.62 (d, J=6.8 Hz, 1H), 7.51 (d, J=7.2 Hz, 1H), 7.49 (m, 2H), 7.45 (s, 2H), 7.33 (d, J=7.2 Hz, 1H), 7.16 (t, J=8.4 Hz, 1H), 6.92 (t, J=8.4 Hz, 1H), 6.79 (d, J=8 Hz, 1H), 5.28 (q, J=8 Hz, 1H), 4.27 (t, J=4.8 Hz, 2H), 2.96 (s, 6H), 2.10 (m, 2H).

### Example 6.1

### N-[(4S)-Chroman-4-yl]-4-(3,5-dichlorophenyl)-8-(dimethylamino)quinoline-7-carboxamide

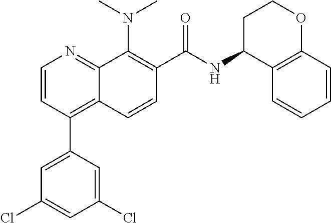 [SMILES: CN(C)c1c(C(=O)N[C@H]2CCOc3ccccc32)ccc2c(-c3cc(Cl)cc(Cl)c3)ccnc12]

A solution of 4-bromoquinoline (0.81 g, 3.89 mmol) in sulfuric acid (17.82 mol/L) in water (10 mL) was treated with nitric acid (70 w/w %) in water (0.30 mL) and was allowed to stir for 1.5 hour at rt. The reaction mixture was diluted with water (200 mL) and was treated slowly with solid K₂CO₃ until effervescence ceased. The aq. phase was extracted with CH₂Cl₂ (3×30 mL). The combined organic layers were dried over anhydrous Na₂SO₄, filtered and concentrated in vacuo. The residue was purified by column chromatography (100% CH₂Cl₂) to give 4-bromo-8-nitro-quinoline. LCMS (method B): Rₜ=1.03 min, m/z=253.0 [M+H]⁺.

A mixture of 4-bromo-8-nitro-quinoline (0.83 g, 3.12 mmol), (3,5-dichlorophenyl) boronic acid (0.61 g, 3.13 mmol), Na₂CO₃ (0.66 g, 6.24 mmol) and 1,1′-bis (diphenyl phosphino) ferrocene-Pd(II)·CH₂Cl₂ complex (0.12 g, 0.16 mmol) was placed under N₂-atmosphere and treated with 1,4-dioxane (15 mL) and de-gassed water (5 mL). The reaction vessel was evacuated and back-filled with N₂ three times before the reaction mixture was warmed to 80° C. and allowed to stir for 30 min. The reaction mixture was allowed to cool to rt before being diluted with water (40 mL) and extracted with CH₂Cl₂ (3×25 mL). The combined organic layers were filtered and concentrated in vacuo. The residues were purified by column chromatography (0-25% EtOAc in cyclohexane) to give 4-(3,5-dichlorophenyl)-8-nitro-quinoline. LCMS (method B): Rₜ=1.37 min, m/z=319.0 [M+H]⁺.

A solution of 4-(3,5-dichlorophenyl)-8-nitro-quinoline (0.99 g, 2.8 mmol) in THF (14 mL) was placed under N₂-atmosphere and treated with EtOH (14 mL), NH₄Cl (0.45 g, 8.4 mmol), iron (0.488 g, 8.56 mmol) and water (7 mL). The resulting reaction mixture was warmed to 80° C. and was allowed to stir for 1 hour. The reaction mixture was cooled to rt before being filtered through Celite® (washing through with CH₂Cl₂). The filtrate was washed with water (30 mL) and the aq. phase was extracted with CH₂Cl₂ (3×20 mL). The combined organic phases were passed through Celite® and concentrated in vacuo. The residue was purified by column chromatography (0-50% EtOAc in cyclohexane) to give 4-(3,5-dichlorophenyl)quinolin-8-amine. LCMS (method B): Rₜ=1.43 min, m/z=289.0 [M+H]⁺.

A solution of 4-(3,5-dichlorophenyl)quinolin-8-amine (0.86 g, 2.52 mmol) in DMF (10 mL) was placed under N₂-atmosphere and cooled over ice before being treated with N-bromosuccinimide (0.45 g, 2.52 mmol). The reaction mixture was stirred for 15 min at 0° C. The reaction mixture was diluted with sat. aq. NaHCO₃ (40 mL) and extracted with CH₂Cl₂ (3×25 mL). The combined organic layers were filtered through Celite® and concentrated in vacuo. The residues were purified by column chromatography (0-20% EtOAc in cyclohexane) to give 7-bromo-4-(3,5-dichlorophenyl) quinolin-8-amine. LCMS (method B): Rₜ=1.64 min, m/z=366.8 [M+H]⁺.

A mixture of 7-bromo-4-(3,5-dichlorophenyl) quinolin-8-amine (0.74 g, 1.9 mmol) and Na₂CO₃ (0.8 g, 5.78 mmol) was placed under N₂-atmosphere and treated with DMF (6.5 mL) and iodomethane (0.40 mL 6.4 mmol). The resulting mixture was warmed to 100° C. and was allowed to stir for 45 min. The reaction mixture was allowed to cool to rt before being diluted with sat. aq. NaHCO₃ (30 mL) and extracted with tert-butylmethyl ether (3×20 mL). The combined organic layers were dried over anhydrous MgSO₄, filtered, and concentrated in vacuo. The residues were purified by column chromatography (0-20% tert-butylmethyl ether in cyclohexane) to give 7-bromo-4-(3,5-dichlorophenyl)-N,N-dimethyl-quinolin-8-amine. LCMS (method B): Rₜ=1.74 min, m/z=395.08 [M+H]⁺.

A solution of 7-bromo-4-(3,5-dichlorophenyl)-N,N-dimethyl-quinolin-8-amine (0.73 g, 1.47 mmol) in 1,4-dioxane (10 mL) was treated with MeOH (10 mL), NEt₃ (0.62 mL, 4.45 mmol) and 1,1′-bis (diphenyl phosphino) ferrocene-Pd(II)—CH₂Cl₂ complex (0.12 g, 0.15 mmol) before being stirred at 100° C. under CO-atmosphere (0.041 g, 1.48 mmol, 56 psi) overnight. The reaction mixture was filtered and concentrated in vacuo. The residue was purified by column chromatography (0-40% EtOAc in cyclohexane) to give methyl 4-(3,5-dichlorophenyl)-8-(dimethylamino)quinoline-7-carboxylate. LCMS (method B): Rₜ=1.57 min, m/z=375.0 [M+H]⁺.

A solution of methyl 4-(3,5-dichlorophenyl)-8-(dimethylamino)quinoline-7-carboxylate (0.46 g, 1.11 mmol) in 1,4-dioxane (15 mL) was treated with water (6 mL) and lithium hydroxide (0.57 g, 22.57 mmol) before being allowed to stir for 1 hour at 100° C. The reaction mixture was concentrated to approximately half volume before being treated with 2 M HCl (to pH 3) and extracted with CH₂Cl₂ (3×15 mL). The combined organic phases were filtered through Celite® and concentrated in vacuo to give 4-(3,5-dichlorophenyl)-8-(dimethylamino)quinoline-7-carboxylic acid. LCMS (method B): Rₜ=1.11 min, m/z=358.8 [M−H]⁻.

A mixture of 4-(3,5-dichlorophenyl)-8-(dimethylamino)quinoline-7-carboxylic acid (0.16 g, 0.36 mmol), (4S)-chroman-4-amine-HCl (0.083 g, 0.434 mmol) and PyBOP (0.29 g, 0.55 mmol) was placed under N₂-atmosphere and treated with THF (0.5 mL) and NEt₃ (0.11 g, 0.15 mL). The resulting reaction mixture was allowed to stir for 30 min at rt. The reaction mixture was poured onto sat. aq. NaHCO₃ (25 mL) and extracted with CH₂Cl₂ (3×15 mL). The combined organic phases were dried over anhydrous Na₂SO₄, filtered through Celite® and concentrated in vacuo. The residue was purified by column chromatography (0-20% EtOAc in cyclohexane) to give the title compound. LCMS (method B): Rₜ=1.66 min, m/z=492.2 [M+H]⁺. ¹H NMR (400 MHz, DMSO-d6) δ[ppm]: 10.37 (d, J=8 Hz, 1H), 9.01 (d, J=4.4 Hz, 1H), 7.94 (d, J=9.2 Hz, 1H), 7.82 (d, J=2 Hz, 1H), 7.61 (m, 3H), 7.55 (d, J=4.4 Hz, 1H), 7.38 (d, J=6.8 Hz, 1H), 7.18 (td, J=8, 1.2 Hz, 1H), 76.99 (td, J=7.6, 0.8 Hz, 1H), 6.81 (dd, J=8, 0.8 Hz, 1H), 5.28 (m, 1H), 4.30 (m, 2H), 3.03 (s, 6H), 2.15 (m, 2H).

### Example 7.1

### N-[(4S)-Chroman-4-yl]-4-(3,5-dichlorophenyl)-8-(dimethylamino)isoquinoline-7-carboxamide

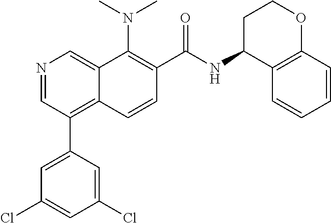 [SMILES: CN(C)c1c(C(=O)N[C@H]2CCOc3ccccc32)ccc2c(-c3cc(Cl)cc(Cl)c3)cncc12]

To a degassed solution of 4-bromo-8-nitro-isoquinoline (864 mg, 3.24 mmol), (3,5-dichlorophenyl) boronic acid (650 mg, 3.34 mmol) and Na₂CO₃ (729 mg, 6.85 mmol) in 1,4-dioxane (15 mL) and water (5 mL) under N₂-atmosphere at rt was added 1,1′-bis(diphenylphosphino) ferrocene-Pd(II)—CH₂Cl₂ complex (255 mg, 0.33 mmol). The mixture was degassed again and put under N₂-atmosphere, then heated to 80° C. for 3 hours. The reaction was cooled to rt, diluted with EtOAc (25 mL) and filtered through a plug of Celite®. The Celite® was washed through with EtOAc (50 mL). The combined organic filtrates were then washed with sat. NaHCO₃ (50 mL) and brine (2×50 mL), then dried over anhydrous Na₂SO₄, filtered and concentrated in vacuo. The residues were purified by column chromatography (10-100% CH₂Cl₂ in cyclohexane to 0-5% MeOH in CH₂Cl₂) to give 4-(3,5-dichlorophenyl)-8-nitro-isoquinoline. LCMS (method B) Rₜ=1.35 min, m/z=319 [M+H]⁺.

A mixture of 4-(3,5-dichlorophenyl)-8-nitro-isoquinoline (881 mg, 2.62 mmol), iron (458 mg, 8.04 mmol) and NH₄Cl (428 mg, 7.97 mmol) under N₂-atmosphere at rt was taken up in THF (13 mL), EtOH (13 mL) and water (6.5 mL) and heated to 75° C. for 1.5 hours. The reaction mixture was cooled to rt, and filtered through a pug of Celite®. The Celite® was then washed through with MeOH (100 mL). The combined filtrates were concentrated in vacuo, then dissolved in a mixture of CH₂Cl₂ (100 mL) and sat. aq. NaHCO₃ (100 mL). The aq. layer was separated and extracted with CH₂Cl₂ (3×50 mL). The combined organic layer was dried over anhydrous Na₂SO₄, filtered and concentrated in vacuo to give 4-(3,5-dichlorophenyl)isoquinolin-8-amine. LCMS (method B) Rₜ=1.24 min, m/z=289 [M+H]⁺.

A solution of 4-(3,5-dichlorophenyl)isoquinolin-8-amine (0.59 g, 1.96 mmol) in DMF (25 mL) under N₂-atmosphere was cooled to −10° C. with an ice/NaCl bath. N-bromosuccinimide (372 mg, 2.03 mmol) was added and the mixture was stirred at rt for 2 hours. The reaction was poured into water (150 mL) and extracted with CH₂Cl₂ (4×100 mL) and 10% MeOH in CH₂Cl₂ (100 mL). The combined organic layers were washed with brine (100 mL), dried over anhydrous Na₂SO₄, filtered and concentrated in vacuo. The residue was taken up in EtOH (30 mL) and heated to 100° C. The mixture was allowed to cool to rt, then filtered to recover the solid. The solid was then dried in vacuo at 60° C. for 1 hour to give 7-bromo-4-(3,5-dichlorophenyl)isoquinolin-8-amine. LCMS (method B) Rₜ=1.42 min, m/z=367 [M+H]⁺.

To a solution of 7-bromo-4-(3,5-dichlorophenyl)isoquinolin-8-amine (132 mg, 0.34 mmol) in formic acid (2.6 mL, 69 mmol) at rt under N₂-atmosphere was added formaldehyde solution (37 wt. % in water) (5 mL, 67 mmol, 37 mass %). The resulting mixture was the heated to 100° C. for 24 hours. Ice (50 g) was added to the reaction, which was then basified with sat. aq. NaHCO₃ (75 mL) and extracted with CH₂Cl₂ (3×50 mL). The combined organic layers were concentrated in vacuo. The oil was purified by column chromatography (5-5% EtOAc in cyclohexane) to give 7-bromo-4-(3,5-dichlorophenyl)-N,N-dimethyl-isoquinolin-8-amine. LCMS (method B) Rₜ=1.67 min, m/z=395 [M+H]⁺.

To a solution of 7-bromo-4-(3,5-dichlorophenyl)-N,N-dimethyl-isoquinolin-8-amine (0.126 g, 0.304 mmol), NEt₃ (0.13 mL, 0.93 mmol) in MeOH (5 mL) and 1,4-dioxane (5 mL) was added 1,1′-bis(diphenylphosphino) ferrocene-Pd(II)—CH₂C₁₂ complex) (45.8 mg, 0.0595 mmol). The reaction was put under CO-atmosphere (0.304 mmol, 42 psi) and heated to 100° C. for 22 hours. The reaction mixture was filtered through a plug of Celite®. The Celite® was washed through with EtOAc (25 mL). The combined organic filtrates were concentrated in vacuo then purified by column chromatography (10-25% EtOAc in cyclohexane) to give methyl 4-(3,5-dichlorophenyl)-8-(dimethylamino)isoquinoline-7-carboxylate. LCMS (method B) Rₜ=1.48 min, m/z=375 [M+H]⁺.

A solution of methyl 4-(3,5-dichlorophenyl)-8-(dimethylamino)isoquinoline-7-carboxylate (71.5 mg, 0.181 mmol) and lithium hydroxide (96.8 mg, 3.84 mmol) in 1,4-dioxane (5 mL) and water (1 mL) under N₂-atmosphere was heated to 100° C. for 2.5 hours. The reaction was cooled to rt and then concentrated in vacuo. The solid was then taken up in water (5 mL) and acidified with 2M HCl (aq., 3.5 mL) to pH=1 and extracted with CH₂Cl₂ (3×15 mL). The combined organic layers were dried over anhydrous Na₂SO₄, filtered and concentrated in vacuo to give 4-(3,5-dichlorophenyl)-8-(dimethylamino)isoquinoline-7-carboxylic acid. LCMS (method B) Rₜ=0.82 min, m/z=361 [M+H]⁺.

A stirring suspension of 4-(3,5-dichlorophenyl)-8-(dimethylamino)isoquinoline-7-carboxylic acid (78.7 mg, 0.218 mmol) in DMF (2.5 mL) was added NEt₃ (0.13 mL, 0.92 mmol), followed by (4S)-chroman-4-amine·HCl (50 mg, 0.27 mmol) and PyBOP (181 mg, 0.34 mmol). The reaction was left to stir at rt under N₂-atmosphere for 2 hours. The reaction was diluted with brine (25 mL) and extracted with CH₂₂ (3×15 mL). The combined organic layers were concentrated in vacuo. The residue was purified by prep-HPLC (Phenomenex Gemini 5 Micron 30*100 mm C-18) (CH₃CN & water adjusted to pH 9 with conc. ammonium hydroxide solution, 30% to 100% CH₃CN over 9 min at 60 ml/min to give the title compound. LCMS (method B) Rₜ=1.41 min m/z=492 [M+H]⁺.

Experimental details for compounds in the tables:

| Ex. | HPLC | NMR |
|---|---|---|
| 1.2 | Rₜ = 1.32; m/z | ¹H-NMR (400 MHz, CDCl₃) δ [ppm]: 8.99 (s, 1 H), 8.51 (d, |
| | (ES+) = 479.2 | J = 6 Hz, 1 H), 7.72 (d, J = 6 Hz, 1 H), 7.22 (t, J = 8 Hz, 2 H), |
| | [M + H]⁺/ | 7.14 (d, J = 7.6 Hz, 1 H), 7.06 (m, 2 H), 6.93 (q, J = 7.6 Hz, 2 |
| | method B | H), 6.88 (d, J = 8 Hz, 1 H), 6.55 (d, J = 7.6 Hz, 1 H), 5.86 (t, J = |
| | | 7.2 Hz, 1 H), 5.39 (q, J = 7.2 Hz, 1 H), 4.36 (m, 1 H), 4.21 (m, |
| | | 1 H), 3.14 (s, 6 H), 3.05 (m, 1 H), 2.9 (m, 1 H), 2.41 (m, 1 H), |
| | | 2.23 (m, 2 H), 2.02 (m, 2 H) |
| 1.3 | Rₜ = 1.32; m/z | ¹H-NMR (400 MHz, CDCl₃) δ [ppm]: 8.99 (s, 1 H), 8.51 (d, |
| | (ES+) = 479.2 | J = 6 Hz, 1 H), 7.72 (d, J = 6 Hz, 1 H), 7.22 (t, J = 8 Hz, 2 H), |
| | [M + H]⁺/ | 7.14 (d, J = 7.6 Hz, 1 H), 7.06 (m, 2 H), 6.93 (q, J = 7.6 Hz, 2 |
| | method B | H), 6.88 (d, J = 8 Hz, 1 H), 6.55 (d, J = 7.6 Hz, 1 H), 5.86 (t, J = |
| | | 7.2 Hz, 1 H), 5.39 (q, J = 7.2 Hz, 1 H), 4.36 (m, 1 H), 4.21 (m, |
| | | 1 H), 3.14 (s, 6 H), 3.05 (m, 1 H), 2.9 (m, 1 H), 2.41 (m, 1 H), |
| | | 2.23 (m, 2 H), 2.02 (m, 2 H) |
| 1.4 | Rₜ = 1.17; m/z | ¹H-NMR (400 MHz, CDCl₃) δ [ppm]: 8.81 (s, 1 H), 8.62 (d, |
| | (ES+) = 477.2 | J = 5 Hz, 1 H), 7.88 (d, J = 6 Hz, 1 H), 7.27 (m, 1 H), 7.20 (m, |
| | [M + H]⁺/ | 1 H), 7.09 (t, J = 7.6 Hz, 1 H), 6.92 (m, 1 H), 6.85 (d, J = 8.4 |
| | method B | Hz, 1 H), 3.674 (d, J = 7.2 Hz, 1 H), 6.41 (d, J = 7.6 Hz, 1 H), |
| | | 6.30 (t, J = 4.4 Hz, 1 H), 5.35 (q, J = 7.2 Hz, 1 H), 4.33 (m, 1 |
| | | H), 4.17 (m, 1 H), 3.17 (s, 6 H), 3.00 (d, J = 8 Hz, 1 H), 2.6 (m, |
| | | 2 H), 2.20 (m, 2 H) |
| 1.5 | Rₜ = 1.43; m/z | ¹H-NMR (400 MHz, DMSO-d₆) δ [ppm]: 9.11 (dd, J = 3.6, 4.8 |
| | (ES+) = 472.2 | Hz, 1 H), 8.45 (s, 1 H), 7.97 (d, J = 5.6 Hz, 1 H), 7.34 (m, 1 |
| | [M + H]⁺/ | Hz), 7.17 (m, 2 H), 6.94 (m, 1 H), 6.80 (dd, J = 2.0, 8 Hz, 1 H), |
| | method B | 5.25 (m, 1 H), 4.26 (m, 3 H), 3.51 (q, J = 8.8 Hz, 1 H), 3.28 |
| | | (m, 1 H), 3.00 (s, 3 H), 2.98 (s, 3 h), 2.73 (d, J = 11.2 Hz, 1 H), |
| | | 1.68-2.51 (m, 7 H), 1.37 (m, 4 H), 1.02 (m, 1 H) |
| 1.6 | Rₜ = 1.45; m/z | ¹H-NMR (400 MHz, DMSO-d₆) δ [ppm]: 9.11 (t, J = 8 Hz, 1 |
| | (ES+)= 472.2 | H), 8.45 (d, J = 4 Hz, 1 H), 7.91 (d, J = 5 Hz, 1 H), 7.35 (t, J = |
| | [M + H]⁺/ | 9.2 Hz, 1 Hz), 7.17 (t, J = 7.6 Hz, 1 H), 6.96 (m, 2 H), 6.80 (d, |
| | method B | J = 8.4 Hz, 1 H), 5.23 (m, 1 H), 4.97 (br s , 1 H), 4.26 (m, 2 H), |
| | | 4.08 (m, 1 H), 3.78 (m, 1 H), 3.17 (d, J = 5.2 Hz, 1 H), 2.97 (s, |
| | | 3 h), 2.96 (s, 3 h), 2.3 (m, 1 H), 2.19 (m, 1 H), 2.03 (m, 2H), |
| | | 1.15-1.86 (m, 8 H) |
| 1.7 | Rₜ = 1.37; m/z | ¹H-NMR (400 MHz, CDCl₃) δ [ppm]: 8.81 (s, 1 H), 8.16 (d, |
| | (ES+) = 500.2 | J = 5.7 Hz, 1 H), 7.38 (d, J = 5.9 Hz, 1 H), 7.30 (d, J = 8.6 Hz, 2 |
| | [M + H]⁺/ | H), 7.22 (m, 1H), 7.16 (m, 2 H), 7.04 (dd, J = 8.8, 2.2 Hz, 1 H), |
| | method B | 6.94 (td, J = 7.4, 1 Hz, 1 H), 6.87 (d, J = 8.2 Hz, 1 H), 5.38 (m, |
| | | 1 H), 4.55 (m, 2 H), 4.36 (m, 1 H), 4.20 (m, 1 H), 3.18 (t, J = |
| | | 8.2 Hz, 2 H), 3.11 (s, 6 H), 2.40 (m, 1 H), 2.21 (m, 1 H) |
| 1.8 | Rₜ = 1.36; m/z | ¹H-NMR (400 MHz, CDCl₃) δ [ppm]: 8.82 (s, 1 H), 8.45 (d, |
| | (ES+) = 498.2 | J = 5.9 Hz, 1 H), 8.04 (d, J = 3.5 Hz, 1 H), 7.92 (d, J = 8.8 Hz, 1 |
| | [M + H]⁺/ | H), 7.78 (d, J = 5.9 Hz, 1 H), 7.61 (d, J = 2.2 Hz, 1 H), 7.28 (d, |
| | method B | J = 7.8 Hz, 1 H), 7.20 (m, 2 H), 6.93 (td, J = 7.4, 0.8 Hz, 1 H), |
| | | 6.86 (d, J = 8.2 Hz, 1 H), 6.78 (d, J = 7.6 Hz, 1 H), 6.64 (d, J = |
| | | 3.3 Hz, 1 H), 5.37 (m, 1 H), 4.34 (m, 1 H), 4.18 (m, 1 H), 3.17 |
| | | (s, 6 H), 2.39 (m, 1 H), 2.19 (m, 1 H) |
| 1.9 | Rₜ = 1.35; m/z | ¹H-NMR (400 MHz, CDCl₃) δ [ppm]: 8.95 (s, 1 H), 8.70 (d, |
| | (ES+) = 535.2 | J = 5.7 Hz, 1 H), 7.99 (d, J = 1.8 Hz, 2 H), 7.94 (d, J = 5.9 Hz, 1 |
| | [M + H]⁺/ | H), 7.47 (t, J = 2 Hz, 1 H), 7.31 (m, 1 H), 7.22 (m, 1 H), 6.96 |
| | method B | (m, 1 H), 6.94 (d, J = 8.2 Hz, 1 H), 6.73 (m, 1 H), 5.40 (m, 1 |
| | | H), 4.38 (m, 1 H), 4.21 (m, 1 H), 3.94 (m, 4 H), 3.44 (m, 4 H), |
| | | 2.43 (m, 1 H), 2.25 (m, 1 H) |
| 1.10 | Rₜ = 1.20; m/z | ¹H-NMR (400 MHz, DMSO-d₆) δ [ppm]: 9.25 (d, J = 8 Hz, 1 |
| | (ES+) = 499.0 | H), 8.74 (s, 1 H), 8.20 (d, J = 5.9 Hz, 1 H), 7.60 (m, 3 H), 7.48 |
| | [M + H]⁺/ | (m, 3 H), 7.40 (d, J = 7.6 Hz, 1 H), 7.19 (td, J = 6.8, 1.6 Hz, 1 |
| | method B | H), 6.96 (td, J = 7.6, 1 Hz, 1 H), 6.82 (d, J = 8.2 Hz, 1 H), 5.27 |
| | | (m, 1 H), 4.27 (m, 2 H), 3.84 (m, 4 H), 3.28 (m, 4 H), 2.24 (m, |
| | | 1 H), 2.08 (m, 1 H) |
| 1.11 | Rₜ = 1.02; m/z | ¹H-NMR (400 MHz, DMSO-d₆) δ [ppm]: 9.27 (d, J = 8 Hz, 1 |
| | (ES+) = 531.0 | H), 8.81 (s, 1 H), 8.60 (d, J = 5.7 Hz, 1 H), 8.26 (d, J = 5.7 Hz, |
| | [M + H]⁺/ | 1 H), 8.04 (m, 2 H), 7.72 (m, 1 H), 7.63 (m, 2 H), 7.39 (d, J = |
| | method B | 7.2 Hz, 1 H), 7.19 (m, 1 H), 6.95 (m, 1 H), 6.81 (d, J = 8.2 Hz, |
| | | 1 H), 5.24 (m, 1 H), 4.25 (m, 2 H), 3.85 (m, 4 H), 3.28 (m, 4 |
| | | H), 2.22 (m, 1 H), 2.06 (m, 1 H) |
| 1.12 | Rₜ = 1.36; m/z | ¹H-NMR (400 MHz, DMSO-d₆) δ [ppm]: 9.00 (d, J = 8 Hz, 1 |
| | (ES+) = 465.0 | H), 8.98 (s, 1 H), 8.64 (d, J = 5.7 Hz, 1 H), 8.60 (br s, 2 H), |
| | [M + H]⁺/ | 8.32 (d, J = 5.7 Hz, 1 H), 8.18 (d, J = 2.1 Hz, 2 H), 7.70 (t, J = 2 |
| | method B | Hz, 1 H), 7.25 (d, J = 7.4 Hz, 1 H), 7.17 (m, 1 H), 6.89 (m, 1 |
| | | H), 6.81 (d, J = 8.2 Hz, 1 H), 5.32 (m, 1 H), 4.29 (m, 2 H), 2.13 |
| | | (m, 2 H) |
| 1.13 | Rₜ = 1.54; m/z | ¹H-NMR (400 MHz, DMSO-d₆) δ [ppm]: 9.31 (d, J = 8 Hz, 1 |
| | (ES+) = | H), 8.98 (s, 1 H), 8.76 (d, J = 5.6 Hz, 1 H), 8.14 (m, 3 H), 7.76 |
| | 508.00 | (t, J = 2 Hz, 1 H), 7.37 (d, J = 8 Hz, 1 H), 7.19 (td, J = 1.6, 8.4 |
| | [M + H]⁺/ | Hz, 1 H), 6.94 (td, J = 1.2, 7.6 Hz, 1 H), 6.81 (dd, J = 1.2, 7.6 |
| | method B | Hz, 1 H), 5.27 (m, 1 H), 4.94 (m, 1 H), 4.27 (m, 2 H), 2.04- |
| | | 2.24 (m, 2 H), 1.32-1.36 (m, 6 H) |
| 1.14 | Rₜ = 1.33; m/z | ¹H-NMR (400 MHz, CDCl₃) δ [ppm]: 9.08 (s, 1 H), 8.65 (d, |
| | (ES+) = 548.0 | J = 5.6 Hz, 1 H), 7.98 (d, J = 2 Hz, 2 H), 7.95 (d, J = 6 Hz, 1 H), |
| | [M + H]⁺/ | 7.53 (br s , 1 H), 7.45 (t, J = 1.6 Hz, 1 H), 7.30 (d, J = 7.6 Hz, 1 |
| | method B | H), 7.22 (t, J = 8.4 Hz , 2 H), 6.95 t, J- 7.6 Hz, 1 H), 6.88 (d, |
| | | J = 8.4 Hz, 1 H), 5.6 (d, J = 6.8 Hz, 1 H), 4.36 (m, 1 H), 4.21 |
| | | (m, 1 H), 3.44 (t, J = 438Hz, 4 H), 2.22-2.6 (m, 6 H), 1.6 (s, 2 |
| | | H) |
| 1.15 | Rₜ = 1.08; m/z | ¹H-NMR (400 MHz, DMSO-d₆) δ [ppm]: 9.15 (d, J = 8.2 Hz, 1 |
| | (ES+) = 461.0 | H), 8.67 (d, J = 6.2 Hz, 1 H), 8.60 (s, 1 H), 8.13 (d, J = 5.9 Hz, |
| | [M + H]⁺/ | 1 H), 7.58 (m, 1 H), 7.35 (d, J = 7.6 Hz, 1 H), 7.25-7.14 (m, 3 |
| | method A | H), 6.91 (t, J = 8 Hz, 1 H), 6.79 (d, J = 8 Hz, 1H), 5.25-5.21 (m, |
| | | 1 H), 4.30-4.19 (m, 2 H), 3.13 (s, 6 H), 2.24-2.16 (m, 1 H), |
| | | 2.07-1.99 (m, 1 H) |
| 1.16 | Rₜ = 1.37; m/z | ¹H-NMR (400 MHz, CDCl₃) δ [ppm]: 8.79 (s, 1 H), 8.51 (d, |
| | (ES+) = 519.2 | J = 5.9 Hz, 1 H), 7.95 (d, J = 2 Hz, 2 H), 7.91 (d, J = 5.9 Hz, 1 |
| | [M + H]⁺/ | H), 7.43 (t, J = 2 Hz, 1 H), 7.29-7.29 (m, 1 H), 7.24-7.20 (m, 1 |
| | method B | H), 6.94 (td, J = 7.5, 1 Hz, 1 H), 6.89-6.85 (m, 2 H), 5.37-5.33 |
| | | (m, 1 H), 4.36 (m, 1 H), 4.23-4.17 (m, 1 H), 3.80-3.73 (m, 4 |
| | | H), 2.42-2.37 (m, 1 H), 2.23-2.17 (m, 1 H), 2.05-2.01 (m, 4 H) |
| 1.17 | Rₜ = 1.40; m/z | ¹H-NMR (400 MHz, DMSO-d₆) δ [ppm]: 9.16 (d, J = 8 Hz, 1 |
| | (ES+) = 511 | H), 8.74 (s, 1 H), 8.65 (d, J = 6 Hz, 1 H), 8.09 (d, J = 2 Hz, 1 |
| | [M + H]⁺/ | H), 8.07 (d, J = 5.6 Hz, 1 H), 7.73 (t, J = 2 Hz, 1 H), 7.04 (t, J = |
| | method B | 6.8 Hz, 1 H), 6.78 (td, J = 2.8, 8.4 Hz, 1 H), 6.67 (dd, J = 2.4, |
| | | 10.4 Hz, 1 H), 5.21 (m, 1H), 4.28 (m, 2 H), 3.1 (s, 6 H), 2.1 |
| | | (m, 2 H) |
| 1.18 | Rₜ = 1.01; m/z | ¹H-NMR (400 MHz, CDCl₃) δ [ppm]: 8.90 (s, 1H), 8.70 (d, J = |
| | (ES+) = 464.0 | 5.9 Hz, 1H), 8.25 (s, 1H), 7.88 (d, J = 5.9 Hz, 1H), 7.53-7.47 |
| | [M + H]⁺/ | (m, 2H), 7.35-7.26 (m, 2H), 7.22-7.18 (m, 2H), 7.00-6.90 (m, |
| | method B | 3H), 6.41 (s, 1H), 5.39-5.35 (m, 1H), 4.36-4.31 (m, 1H), 4.21- |
| | | 4.15 (m, 1H), 3.18 (s, 6H), 2.42-2.35 (m, 1H), 2.23-2.14 (m, |
| | | 1H) |
| 1.19 | Rₜ = 1.49; m/z | ¹H-NMR (400 MHz, CDCl₃) δ [ppm]: 9.35 (s, 1 H), 8.66 (d, |
| | (ES+) = 526.0 | J = 5.9 Hz, 1 H), 8.07 (d, J = 1.8 Hz, 2 H), 7.58-7.55 (m, 3 H), |
| | [M + H]⁺/ | 7.50 (t, J = 1.9 Hz, 1 H), 7.45-7.41 (m, 3 H), 7.15-7.11 (m, 1 |
| | method B | H), 6.80-6.71 (m, 3 H), 5.63-5.59 (m, 1 H), 5.11 (dt, J = 7.6, |
| | | 5.0 Hz, 1 H), 4.07 (m, 1 H), 3.59-3.53 (m, 1 H), 2.12-2.05 (m, |
| | | 1 H), 1.76-1.71 (m, 1 H) |
| 1.20 | Rₜ = 1.42; m/z | ¹H-NMR (400 MHz, CDCl₃) δ [ppm]: 9.00 (s, 1 H), 8.76 (d, |
| | (ES+) = 490.0 | J = 5.9 Hz, 1 H), 8.25 (d, J = 5.7 Hz, 1 H), 7.99 (d, J = 1.8 Hz, 2 |
| | [M + H]⁺/ | H), 7.47 (t, J = 1.9 Hz, 1 H), 7.31 (d, J = 7.6 Hz, 1 H), 7.23- |
| | method B | 7.19 (m, 1 H), 6.96-6.92 (m, 1 H), 6.86 (d, J = 8.2 Hz, 1 H), |
| | | 6.29 (d, J = 7.4 Hz, 1 H), 5.44-5.39 (m, 1 H), 4.40-4.35 (m, 1 |
| | | H), 4.24-4.18 (m, 1 H), 2.47-2.40 (m, 1 H), 2.33-2.26 (m, 2 |
| | | H), 1.34-1.25 (m, 2 H), 0.88-0.80 (m, 2 H) |
| 1.21 | Rₜ = 1.45; m/z | ¹H NMR (400 MHz, CDCl₃) δ [ppm]: 8.89 (s, 1H), 8.63 (d, J = |
| | (ES+) = 477.0 | 5.9 Hz, 1H), 7.98 (d, J = 2 Hz, 2H), 7.91 (d, J = 5.9 Hz, 1H), |
| | [M + H]⁺/ | 7.45 (t, J = 2 Hz, 1H), 7.39 (d, J = 6.6 Hz, 1H), 7.30-7.22 (m, |
| | method B | 3H), 6.52 (d, J = 8.4 Hz, 1H), 5.73 (q, J = 7.5 Hz, 1H), 3.19 (s, |
| | | 6H), 3.10-3.02 (m, 2H), 2.81-2.74 (m, 1H), 2.04-1.95 (m, 1H) |
| 1.22 | Rₜ = 1.35; m/z | ¹H NMR (400 MHz, CDCl₃) δ [ppm]: 8.93 (s, 1H), 8.65 (d, J = |
| | (ES+) = 518.0 | 5.9 Hz, 1H), 7.98 (d, J = 2 Hz, 2H), 7.91 (d, J = 5.7 Hz, 1H), |
| | [M + H]⁺/ | 7.65 (d, J = 1.8 Hz, 1H), 7.50-7.45 (m, 2H), 7.04-7.00 (m, 1H), |
| | method B | 6.95 (d, J = 8.6 Hz, 1H), 5.44-5.39 (m, 1H), 4.48-4.33 (m, 2H), |
| | | 3.18 (s, 6H), 2.45-2.38 (m, 2H) |
| 1.23 | Rₜ = 1.48; m/z | ¹H NMR (400 MHz, CDCl₃) δ [ppm]: 8.89 (s, 1H), 8.63 (d, J = |
| | (ES+) = 527.0 | 5.9 Hz, 1H), 7.97 (d, J = 2 Hz, 2H), 7.89 (d, J = 5.9 Hz, 1H), |
| | [M + H]⁺/ | 7.45 (t, J = 1.9 Hz, 1H), 7.23 (d, J = 8.2 Hz, 1H), 6.94-6.85 (m, |
| | method B | 3H), 5.38-5.33 (m, 1H), 4.36 (ddd, J = 11.2, 6.1, 3.5 Hz, 1H), |
| | | 4.23-4.17 (m, 1H), 3.16 (s, 6H), 2.42-2.35 (m, 1H), 2.23-2.17 |
| | | (m, 1H) |
| 1.24 | Rₜ = 1.11; m/z | ¹H NMR (400 MHz, DMSO-d₆) δ [ppm]: 9.15 (d, J = 8.2 Hz, |
| | (ES+) = 491.2 | 1H), 8.66 (d, J = 5.9 Hz, 1H), 8.60 (s, 1H), 8.12 (d, J = 5.9 Hz, |
| | [M + H]⁺/ | 1H), 7.35-7.27 (m, 2H), 7.18-7.12 (m, 2H), 6.93-6.89 (m, 1H), |
| | method B | 6.79 (d, J = 8.2 Hz, 1H), 5.25-5.21 (m, 1H), 4.30-4.24 (m, 2H), |
| | | 3.89 (d, J = 3.7 Hz, 3H), 3.13 (s, 6H), 2.24-2.16 (m, 1H), 2.07- |
| | | 2.00 (m, 1H) |
| 1.25a | Rₜ = 1.38; m/z | ¹H NMR (400 MHz, CDCl₃) δ [ppm]: 8.88 (s, 1 H), 8.63 (d, |
| | (ES+) = 523.2 | J = 5.9 Hz, 1 H), 7.97 (d, J = 1.8 Hz, 2 H), 7.90 (d, J = 5.9 Hz, 1 |
| | [M + H]⁺/ | H), 7.45 (t, J = 1.9 Hz, 1 H), 7.19 (d, J = 8.6 Hz, 1 H), 6.69 (d, |
| | method B | J = 7.2 Hz, 1 H), 6.54 (dd, J = 2.5, 8.6 Hz, 1 H), 6.40 (d, J = 2.5 |
| | | Hz, 1 H), 5.32-5.28 (m, 1 H), 4.38-4.32 (m, 1 H), 4.20-4.14 |
| | | (m, 1 H), 3.77 (s, 3 H), 3.17 (s, 6 H), 2.41-2.34 (m, 1 H), 2.24- |
| | | 2.19 (m, 1 H) |
| 1.25b | Rₜ = 1.38; m/z | ¹H NMR (400 MHz, CDCl₃) δ [ppm]: 8.88 (s, 1 H), 8.63 (d, |
| | (ES+) = 523.2 | J = 5.9 Hz, 1 H), 7.97 (d, J = 1.8 Hz, 2 H), 7.90 (d, J = 5.9 Hz, 1 |
| | [M + H]⁺/ | H), 7.45 (t, J = 1.9 Hz, 1 H), 7.19 (d, J = 8.6 Hz, 1 H), 6.69 (d, |
| | method B | J = 7.2 Hz, 1 H), 6.54 (dd, J = 2.5, 8.6 Hz, 1 H), 6.40 (d, J = 2.5 |
| | | Hz, 1 H), 5.32-5.28 (m, 1 H), 4.38-4.32 (m, 1 H), 4.20-4.14 |
| | | (m, 1 H), 3.77 (s, 3 H), 3.17 (s, 6 H), 2.41-2.34 (m, 1 H), 2.24- |
| | | 2.19 (m, 1 H) |
| 1.26a | Rt= 1.43; m/z | ¹H NMR (400 MHz, CDCl₃) δ [ppm]: 8.91 (s, 1H), 8.63 (d, J = |
| | (ES+)= 507.0 | 5.9 Hz, 1H), 7.97 (d, J = 2 Hz, 2H), 7.88 (d, J = 5.7 Hz, 1H), |
| | [M+H]⁺/ | 7.45-7.41 (m, 2H), 7.24-7.19 (m, 1H), 6.98-6.94 (m, 1H), 6.88 |
| | method B | (dd, J = 0.9, 8.3 Hz, 1H), 6.75 (s, 1H), 4.31-4.28 (m, 2H), 3.13 |
| | | (s, 6H), 3.08-3.03 (m, 1H), 2.30-2.23 (m, 1H), 2.01-1.99 (m, |
| | | 3H) |
| 1.26b | Rₜ = 1.43; m/z | ¹H NMR (400 MHz, CDCl₃) δ [ppm]: 8.91 (s, 1H), 8.63 (d, J = |
| | (ES+) = 507.0 | 5.9 Hz, 1H), 7.97 (d, J = 2 Hz, 2H), 7.88 (d, J = 5.7 Hz, 1H), |
| | [M + H]⁺/ | 7.45-7.41 (m, 2H), 7.24-7.19 (m, 1H), 6.98-6.94 (m, 1H), 6.88 |
| | method B | (dd, J = 0.9, 8.3 Hz, 1H), 6.75 (s, 1H), 4.31-4.28 (m, 2H), 3.13 |
| | | (s, 6H), 3.08-3.03 (m, 1H), 2.30-2.23 (m, 1H), 2.01-1.99 (m, |
| | | 3H) |
| 1.27a | Rₜ = 1.25; m/z | ¹H NMR (400 MHz, CDCl₃) δ [ppm]: 8.90 (s, 1H), 8.65 (d, J = |
| | (ES+) = 492.0 | 5.7 Hz, 1H), 8.49 (dd, J = 1.4, 4.7 Hz, 1H), 7.98 (d, J = 2 Hz, |
| | [M + H]⁺/ | 2H), 7.91 (d, J = 5.7 Hz, 1H), 7.75 (d, J = 7.8 Hz, 1H), 7.47- |
| | method B | 7.44 (m, 1H), 7.18 (dd, J = 4.7, 7.8 Hz, 1H), 6.69-6.65 (m, |
| | | 1H), 5.53-5.48 (m, 1H), 3.18 (s, 6H), 3.07-2.96 (m, 2H), 2.28- |
| | | 2.22 (m, 1H), 2.07-1.97 (m, 3H) |
| 1.27b | Rₜ = 1.25; m/z | ¹H NMR (400 MHz, CDCl₃) δ [ppm]: 8.90 (s, 1H), 8.65 (d, J = |
| | (ES+) = 492.0 | 5.7 Hz, 1H), 8.49 (dd, J = 1.4, 4.7 Hz, 1H), 7.98 (d, J = 2 Hz, |
| | [M + H]⁺/ | 2H), 7.91 (d, J = 5.7 Hz, 1H), 7.75 (d, J = 7.8 Hz, 1H), 7.47- |
| | method B | 7.44 (m, 1H), 7.18 (dd, J = 4.7, 7.8 Hz, 1H), 6.69-6.65 (m, |
| | | 1H), 5.53-5.48 (m, 1H), 3.18 (s, 6H), 3.07-2.96 (m, 2H), 2.28- |
| | | 2.22 (m, 1H), 2.07-1.97 (m, 3H) |
| 1.28 | Rₜ = 1.14; m/z | ¹H NMR (400 MHz, DMSO-d₆) δ [ppm]: 9.16 (d, J = 8 Hz, |
| | (ES+) = 480.2 | 1H), 8.61-8.58 (m, 2H), 7.98 (d, J = 5.9 Hz, 1H), 7.35 (d, J = |
| | [M + H]⁺/ | 7.4 Hz, 1H), 7.20-7.09 (m, 2H), 6.92 (t, J = 7.3 Hz, 1H), 6.81- |
| | method B | 6.78 (m, 2H), 6.59 (d, J = 7.8 Hz, 1H), 5.27-5.22 (m, 1H), |
| | | 4.30-4.20 (m, 2H), 3.18 (t, J = 8.1 Hz, 2H), 3.10 (s, 6H), 2.74 |
| | | (s, 3H), 2.66 (t, J = 8 Hz, 2H), 2.23-2.18 (m, 1H), 2.10-2.03 |
| | | (m, 1H) |
| 1.29 | Rₜ = 1.07; m/z | ¹H NMR (400 MHz, CDCl₃) δ [ppm]: 8.79 (s, 1H), 8.68 (d, J = |
| | (ES+) = 494.0 | 5.9 Hz, 1H), 8.51 (s, 1H), 8.00 (d, J = 5.9 Hz, 1H), 7.86 (d, J = |
| | [M + H]⁺/ | 2.1 Hz, 1H), 7.26 (m, 2H), 6.95-6.91 (m, 1H), 6.86 (d, J = 8.2 |
| | method A | Hz, 1H), 6.81 (s, 1H), 5.40-5.35 (m, 1H), 4.37-4.32 (m, 1H), |
| | | 4.22-4.16 (m, 1H), 3.19 (s, 6H), 2.43-2.36 (m, 1H), 2.22-2.17 |
| | | (m, 1H) |
| 1.30 | Rₜ = 1.43; m/z | ¹H NMR (400 MHz, CDCl₃) δ [ppm]: 9.17 (s, 1 H), 8.85 (d, |
| | (ES+)-484.0 | J = 5.7 Hz, 1 H), 8.10 (d, J = 5.7 Hz, 1 H), 8.03 (d, J = 1.8 Hz, 2 |
| | [M + H]⁺/ | H), 7.50 (t, J = 1.9 Hz, 1 H), 7.34 (d, J = 7.8 Hz, 1 H), 7.25- |
| | method B | 7.21 (m, 1 H), 6.96 (td, J = 7.5, 0.9 Hz, 1 H), 6.89-6.87 (m, 1 |
| | | H), 6.55 (d, J = 7.4 Hz, 1 H), 5.46-5.42 (m, 1 H), 4.40-4.35 (m, |
| | | 1 H), 4.27-4.21 (m, 1 H), 2.46-2.40 (m, 1 H), 2.33-2.28 (m, 1 |
| | | H) |
| 1.31 | Rₜ = 1.38; m/z | ¹H NMR (400 MHz, CDCl₃) δ [ppm]: 9.15 (s, 1H), 8.70 (d, J = |
| | (ES+) = 509.0 | 5.9 Hz, 1H), 8.09 (d, J = 5.7 Hz, 1H), 8.00 (d, J = 1.8 Hz, 2H), |
| | [M + H]⁺/ | 7.47 (t, J = 2 Hz, 1H), 7.36-7.34 (m, 1H), 7.24-7.19 (m, 2H), |
| | method B | 6.95 (td, J = 7.5, 0.9 Hz, 1H), 6.87 (d, J = 8.4 Hz, 1H), 5.40- |
| | | 5.36 (m, 1H), 4.37 (ddd, J = 11, 5.8, 3.5 Hz, 1H), 4.25-4.19 (m, |
| | | 1H), 3.59 (s, 3H), 3.33 (s, 3H), 2.43-2.36 (m, 1H), 2.28-2.23 |
| | | (m, 1H) |
| 1.32 | Rₜ = 1.48; m/z | ¹H NMR (400 MHz, CDCl₃) δ [ppm]: 8.86 (s, 1H), 8.63 (d, J = |
| | (ES+) = 491.0 | 5.9 Hz, 1H), 7.97 (d, J = 2 Hz, 2H), 7.90 (d, J = 5.9 Hz, 1H), |
| | [M + H]⁺/ | 7.44 (t, J = 2 Hz, 1H), 7.40-7.36 (m, 1H), 7.24-7.20 (m, 2H), |
| | method B | 7.16-7.13 (m, 1H), 6.51 (d, J = 8.2 Hz, 1H), 5.42 (dt, J = 8, 5.6 |
| | | Hz, 1H), 3.19 (s, 6H), 2.89-2.82 (m, 2H), 2.24-2.18 (m, 1H), |
| | | 2.07-2.00 (m, 3H) |
| 1.33 | Rₜ = 1.43; m/z | ¹H NMR (400 MHz, DMSO-d₆) δ [ppm]: 9.37 (d, J = 8 Hz, |
| | (ES+) = 480.0 | 1H), 8.94 (s, 1H), 8.76 (d, J = 5.7 Hz, 1H), 8.15-8.14 (m, 3H), |
| | [M + H]⁺/ | 7.76 (t, J = 2 Hz, 1H), 7.39 (d, J = 7 Hz, 1H), 7.21-7.17 (m, |
| | method B | 1H), 6.96-6.92 (m, 1H), 6.81 (d, J = 8.2 Hz, 1H), 5.31-5.26 (m, |
| | | 1H), 4.22 (s, 5H), 2.26-2.12 (m, 2H) |
| 1.34 | Rₜ = 1.44; m/z | ¹H NMR (400 MHz, CDCl₃) δ [ppm]: 8.87 (s, 1H), 8.62 (d, J = |
| | (ES+) = 477.0 | 5.9 Hz, 1H), 7.98 (d, J = 1.8 Hz, 2H), 7.90 (d, J = 5.9 Hz, 1H), |
| | [M + H]⁺/ | 7.44 (t, J = 2 Hz, 1H), 7.39 (d, J = 6.6 Hz, 1H), 7.29-7.26 (m, |
| | method B | 3H), 6.55 (d, J = 8.2 Hz, 1H), 5.72 (q, J = 7.5 Hz, 1H), 3.18 (s, |
| | | 6H), 3.10-3.00 (m, 2H), 2.80-2.73 (m, 1H), 2.04-1.92 (m, 1H) |
| 1.35 | Rₜ = 1.48; m/z | ¹H NMR (400 MHz, CDCl₃) δ [ppm]: 8.85 (s, 1H), 8.62 (d, J = |
| | (ES+) = 491.0 | 5.9 Hz, 1H), 7.97 (d, J = 2 Hz, 2H), 7.90 (d, J = 5.9 Hz, 1H), |
| | [M + H]⁺/ | 7.44 (t, J = 2 Hz, 1H), 7.40-7.36 (m, 1H), 7.24-7.20 (m, 2H), |
| | method B | 7.16-7.12 (m, 1H), 6.52 (d, J = 8.2 Hz, 1H), 5.42 (dt, J = 8.2, |
| | | 5.5 Hz, 1H), 2.89-2.81 (m, 2H), 2.24-2.18 (m, 1H), 2.07-1.99 |
| | | (m, 3H) |
| 1.36a | Rₜ = 1.20; m/z | ¹H NMR (400 MHz, CDCl₃) δ [ppm]: 8.92 (s, 1H), 8.65 (d, J = |
| | (ES+) = 478.0 | 5.9 Hz, 1H), 8.51 (d, J = 4.7 Hz, 1H), 7.99 (d, J = 1.8 Hz, 2H), |
| | [M + H]⁺/ | 7.91 (d, J = 5.9 Hz, 1H), 7.77-7.74 (m, 1H), 7.45 (t, J = 2 Hz, |
| | method B | 1H), 7.22-7.17 (m, 1H), 6.71-6.66 (m, 1H), 5.78 (q, J = 7.5 Hz, |
| | | 1H), 3.19 (m, 8H), 2.87-2.80 (m, 1H), 2.07-1.99 (m, 1H) |
| 1.36b | Rₜ = 1.20; m/z | ¹H NMR (400 MHz, CDCl₃) δ [ppm]: 8.92 (s, 1H), 8.65 (d, J = |
| | (ES+) = 478.0 | 5.9 Hz, 1H), 8.51 (d, J = 4.7 Hz, 1H), 7.99 (d, J = 1.8 Hz, 2H), |
| | [M + H]⁺/ | 7.91 (d, J = 5.9 Hz, 1H), 7.77-7.74 (m, 1H), 7.45 (t, J = 2 Hz, |
| | method B | 1H), 7.22-7.17 (m, 1H), 6.71-6.66 (m, 1H), 5.78 (q, J = 7.5 Hz, |
| | | 1H), 3.19 (m, 8H), 2.87-2.80 (m, 1H), 2.07-1.99 (m, 1H) |
| 1.37 | Rₜ = 1.13; m/z | ¹H NMR (400 MHz, CDCl₃) δ [ppm]: 9.05 (s, 1 H), 8.71 (d, |
| | (ES+) = 534 | J = 5.9 Hz, 1 H), 7.99 (d, J = 2 Hz, 2 H), 7.85 (d, J = 5.9 Hz, 1 |
| | [M + H]⁺/ | H), 7.47 (t, J = 2 Hz, 1 H), 7.30 (d, J = 7.8 Hz, 1 H), 7.24-7.21 |
| | method B | (m, 1 H), 7.00-6.94 (m, 2 H), 6.89 (d, J = 8.4 Hz, 1 H), 6.26 (s, |
| | | 1 H), 5.42-5.37 (m, 1 H), 5.06 (s, 2 H), 4.40-4.34 (m, 1 H), |
| | | 4.24-4.18 (m, 1 H), 4.12-4.11 (m, 2 H), 2.47-2.40 (m, 1 H), |
| | | 2.25-2.19 (m, 1 H) |
| 1.38 | Rₜ = 1.36; m/z | ¹H NMR (400 MHz, DMSO d6-) δ [ppm]: 9.51 (d, J = 7.8 Hz, |
| | (ES+) = 489.0 | 1 H), 9.19 (s, 1 H), 8.91 (d, J = 5.9 Hz, 1 H), 8.32 (d, J = 5.9 |
| | [M + H]⁺/ | Hz, 1 H), 8.09 (d, J = 2 Hz, 2 H), 7.79 (t, J = 2 Hz, 1 H), 7.42 |
| | method B | (d, J = 7 Hz, 1 H), 7.21-7.17 (m, 1 H), 6.94-6.91 (m, 1 H), 6.82 |
| | | (d, J = 8 Hz, 1 H), 5.33-5.28 (m, 1 H), 4.71 (s, 2 H), 4.33-4.23 |
| | | (m, 2 H), 2.28-2.21 (m, 2 H) |
| 1.39 | Rₜ = 1.44; m/z | ¹H NMR (400 MHz, CDCl₃) δ [ppm]: 8.92-8.91 (m, 1H), |
| | (ES+) = 507 | 8.65-8.63 (m, 1 H), 7.97-7.96 (m, 2 H), 7.92-7.90 (m, 1 H), |
| | [M + H]⁺/ | 7.45 (t, J = 1.9 Hz, 1 H), 7.29 (d, J = 7.8 Hz, 1 H), 7.24-7.20 |
| | method A | (m, 1 H), 7.00-6.93 (m, 1 H), 6.87 (d, J = 8.2 Hz, 1 H), 6.70- |
| | | 6.61 (m, 1 H), 5.52 (dd, J = 4.8, 9.1 Hz, 0.4 H), 5.09 (dd, J = |
| | | 6.5, 7.5 Hz, 0.6 H), 4.26-4.19 (m, 1 H), 4.08-4.03 (m, 0.6 H), |
| | | 3.86 (dd, J = 9.6, 11.1 Hz, 0.4 H), 3.19-3.18 (m, 6 H), 2.57- |
| | | 2.53 (m, 0.4 H), 2.35-2.29 (m, 0.6 H), 1.20-1.15 (m, 3 H) |
| 1.40 | Rₜ = 1.55; m/z | ¹H NMR (400 MHz, CDCl₃) δ [ppm]: 9.51 (s, 1 H), 8.89-8.84 |
| | (ES⁺) = 475.0 | (m, 2 H), 8.43 (s, 1 H), 8.05 (d, J = 1.8 Hz, 2 H), 7.51 (t, J = 2 |
| | [M + H]⁺/ | Hz, 1 H), 7.26 (d, J = 15.8 Hz, 1 H), 7.03 (d, J = 7.6 Hz, 1 H), |
| | method B | 6.98 (d, J = 8.4 Hz, 1 H), 6.91 (t, J- 7.5 Hz, 1 H), 5.91 (dd, J = |
| | | 7, 10.3 Hz, 1 H), 4.49 (dt, J = 11.5, 3.5 Hz, 1 H), 4.33 (td, J = |
| | | 11.5, 2 Hz, 1 H), 2.56-2.46 (m, 1 H), 2.37-2.29 (m, 1 H) |
| 1.41 | Rₜ = 1.16; m/z | ¹H NMR (400 MHz, CDCl₃) δ [ppm]: 8.97 (s, 1H), 7.96 (d, J = |
| | (ES+) = 477.0 | 5.9 Hz, 1H), 7.57 (d, J = 6 Hz, 1H), 7.31 (d, J = 7.6 Hz, 1H), |
| | [M + H]⁺/ | 7.24-7.19 (m, 2H), 7.07-6.97 (m, 5H), 5.41-5.37 (m, 1H), |
| | method B | 4.39-4.23 (m, 2H), 3.14 (s, 6H), 2.46-2.39 (m, 2H) |
| 1.42 | Rₜ = 1.45; | ¹H NMR (400 MHz, DMSO-d6) δ [ppm]: 9.24 (d, J = 8.2 Hz, |
| | m/z (ES+) = | 1H), 8.94 (s, 1H), 8.75 (d, J = 5.9 Hz, 1H), 8.31 (d, J = 5.9 Hz, |
| | 492.2 | 1H), 8.04 (d, J = 2 Hz, 2H), 7.76 (t, J = 2 Hz, 1H), 7.38 (d, J = 7 |
| | [M + H]⁺/ | Hz, 1H), 7.18 (td, J = 8.4, 1.2 Hz, 1H), 6.94 (td, J = 7.5, 1 Hz, |
| | method B | 1H), 6.80 (dd, J = 0.8, 8.2 Hz, 1H), 5.31-5.26 (m, 1H), 4.32- |
| | | 4.25 (m, 2H), 3.86-3.79 (m, 1H), 2.26-2.19 (m, 2H), 1.49-1.58 |
| | | (m, 6H) |
| 1.43 | Rₜ = 1.06; | ¹H NMR (400 MHz, CDCl₃) δ [ppm]: 8.89 (s, 1H), 8.71 (d, J = |
| | m/z (ES+) = | 5.9 Hz, 1H), 7.98 (d, J = 5.9 Hz, 1H), 7.30-7.18 (m, 2H), 7.07- |
| | 504.2 | 7.05 (m, 1H), 6.96-6.91 (m, 2H), 6.86 (d, J = 8.2 Hz, 1H), |
| | [M + H]⁺/ | 6.77-6.73 (m, 1H), 5.39-5.35 (m, 1H), 4.37-4.32 (m, 1H), |
| | method A | 4.21-4.15 (m, 1H), 3.19 (s, 6H), 2.85-2.83 (m, 6H), 2.43-2.36 |
| | | (m, 1H), 2.24-2.19 (m, 1H) |
| 1.44 | Rₜ = 1.40; | ¹H NMR (400 MHz, CDCl₃) δ [ppm]: 9.10 (s, 1H), 8.68 (d, J = |
| | m/z (ES+) = | 5.9 Hz, 1H), 8.22 (s, 1H), 8.01 (d, J = 1.8 Hz, 2H), 7.94 (d, J = |
| | 493.0 | 5.9 Hz, 1H), 7.56-7.52 (m, 1H), 7.46 (t, J = 1.9 Hz, 1H), 7.19 |
| | [M + H]⁺/ | (t, J = 8.2 Hz, 1H), 6.76 (d, J = 8.2 Hz, 1H), 4.20-4.18 (m, 2H), |
| | method B | 3.24 (s, 6H), 2.72 (t, J = 6.5 Hz, 2H), 2.11-2.05 (m, 2H) |
| 1.45 | Rₜ = 1.44; | ¹H NMR (400 MHz, DMSO-d6) δ [ppm]: 9.13 (d, J = 8.2 Hz, |
| | m/z (ES+) = | 1H), 8.72 (s, 1H), 8.65 (d, J = 5.9 Hz, 1H), 8.09 (d, J = 2 Hz, |
| | 507.0 | 2H), 8.06 (d, J = 5.7 Hz, 1H), 7.73 (t, J = 2 Hz, 1H), 7.25 (d, J = |
| | [M + H]⁺/ | 7.8 Hz, 1H), 6.75 (dd, J = 1, 7.8 Hz, 1H), 6.62 (s, 1H), 5.22- |
| | method B | 5.17 (m, 1H), 4.28-4.19 (m, 2H), 3.10 (s, 6H), 2.00-2.27 (m, |
| | | 5H) |
| 1.46 | Rₜ = 1.37; | ¹H NMR (400 MHz, CDCl₃) δ [ppm]: 8.89 (s, 1 H), 8.64 (d, |
| | m/z (ES+) = | J = 6 Hz, 1 H), 7.98 (d, J = 1.6 Hz, 1 H), 7.91 (d, J = 5.6 Hz, 1 |
| | 499.0 | H), 7.45 (t, J = 2 Hz, 1 H), 7.16 (d, J = 5.2 Hz, 1 H), 6.77 (d, J = |
| | [M + H]⁺/ | 7.2 Hz, 1 H), 6.65 (d, J = 5.6 Hz, 1 H), 5.4 (m, 1 H), 4.37 (m, 1 |
| | Method B | H), 4.17 (m, 1 H), 3.2 (s, 6 H), 2.45 (m, 1H), 2.22 (m, 1 H) |
| 1.47 | Rₜ = 1.37; | ¹H NMR (400 MHz, CDCl₃) δ [ppm]: 8.89 (s, 1 H), 8.64 (d, |
| | m/z (ES+) = | J = 6 Hz, 1 H), 7.98 (d, J = 1.6 Hz, 1 H), 7.91 (d, J = 5.6 Hz, 1 |
| | 499.0 | H), 7.45 (t, J = 2 Hz, 1 H), 7.16 (d, J = 5.2 Hz, 1 H), 6.77 (d, J = |
| | [M + H]⁺/ | 7.2 Hz, 1 H), 6.65 (d, J = 5.6 Hz, 1 H), 5.4 (m, 1 H), 4.37 (m, 1 |
| | Method B | H), 4.17 (m, 1 H), 3.2 (s, 6 H), 2.45 (m, 1H), 2.22 (m, 1 H) |
| 1.48 | Rₜ = 1.20; | ¹H NMR (400 MHz, DMSO-d6) δ [ppm]: 9.19 (d, J = 8.2 Hz, 1 |
| | m/z (ES+) = | H), 8.73 (s, 1 H), 8.65 (d, J = 5.7 Hz, 1 H), 8.06 (d, J = 5.9 Hz, 1 |
| | 461.2 | H), 7.84-7.76 (m, 2 H), 7.41-7.33 (m, 2 H), 7.21-7.15 (m, 1 |
| | [M + H]⁺/ | H), 6.96-6.90 (m, 1 H), 6.80 (dd, J = 8.2, 1 Hz, 1H), 5.30-5.22 |
| | Method B | (m, 1 H), 4.33-4.18 (m, 2 H), 1.24 (s, 6 H), 2.27-2.16 (m, 1 |
| | | H), 2.11-2.02 (m, 1 H) |
| 1.49 | Rₜ = 1.07; | ¹H NMR (400 MHz, CDCl₃) δ [ppm]: 8.90 (s, 1 H), 8.67 (d, |
| | m/z (ES+) = | J = 5.9 Hz, 1 H), 7.93 (d, J = 5.9 Hz, 1 H), 7.64-7.57 (m, 1 H), |
| | 443.2 | 7.50-7.43 (m, 1 H), 7.32-7.25 (m, 2 H), 7.24-7.16 (m, 2 H), |
| | [M + H]⁺/ | 6.96-6.90 (m, 1 H), 6.86 (d, J = 8.4 Hz, 1 H), 6.8 (d, J = 7.6 Hz, |
| | Method B | 1 H), 5.37 (q, J = 7.2 Hz, 1 H), 4.38-4.30 (m, 1 H), 4.22-4.14 |
| | | (m, 1H), 3.18 (s, 6 H), 2.43-2.34 (m, 1H), 2.24-2.15 (m, 1 H) |
| 1.50 | Rₜ = 1.19; | ¹H NMR (400 MHz, CDCl₃) δ [ppm]: 8.86 (s, 1H), 8.67 (d, J = |
| | m/z (ES+) = | 6 Hz, 1 H), 7.98 (d, J = 6 Hz, 1 H), 7.26-7.29 (m, 2 H), 7.21 |
| | 479.0 | (td, J = 1.2, 8.4 Hz, 1 H), 7.01-7.15 (m, 1 H), 6.93 (td, J = 1.2, |
| | [M + H]⁺/ | 8.4 Hz, 1 H), 6.87 (dd, J = 0.4, 8 Hz, 1 H) 6.66 (d, J = 7.6 Hz, |
| | Method B | 1 H), 5.37 (q, J = 5.2 Hz, 1 H), 4.32-4.38 (m , 1 H), 4.16-4.22 |
| | | (m, 1 H), 3.2 (s, 6 H), 2.38-2.46 (m, 1 H), 2.19-2.26 (m, 1 H) |
| 1.51 | Rₜ = 1.30; | ¹H NMR (400 MHz, CDCl₃) δ [ppm]: 8.90 (s, 1 H), 8.63 (d, |
| | m/z (ES+) = | J = 5.6 Hz, 1 H), 7.89 (t, J = 6 Hz, 2 H), 7.86 (d, J = 6.8 Hz, 1 |
| | 479.0 | H), 7.30 (d, J = 7.6 Hz, 1 H), 7.23 (t, J = 8.4 Hz, 1 H), 6.95 (td, |
| | [M + H]⁺/ | J = 1.2, 7.6 Hz, 1 H), 6.88 (d, J = 8.4 Hz, 1 H), 6.73 (d, J = 7.2 |
| | Method B | Hz, 1 H), 5.39 (q, J = 7.2 Hz, 1 H), 4.18-4.39 (m, 2 H), 3.18 (s, |
| | | 6 H), 2.19-2.45 (m, 2 H) |
| 1.52 | Rₜ = 1.43; | ¹H NMR (400 MHz, DMSO-d6) δ [ppm]: 9.21 (d, J = 8 Hz, 1 |
| | m/z (ES−) = | H), 8.76 (s, 2 H), 8.72 (s, 1 H), 8.71 (d, J = 6 Hz, 1 H), 8.27 (s, |
| | 559.2 [M − H]⁻/ | 1 H), 8.12 (d, J = 5.6 Hz, 1 H), 7.38 (d, J = 7.6 Hz, 1 H), 7.18 |
| | Method B | (td, J = 1.6, 8.4 Hz, 1 H), 6.93 (td, J = 1.2, 8.4 Hz, 1 H), 6.80 |
| | | (dd, J = 0.8, 8 Hz, 1 H), 5.24 (q, J = 7.6 Hz, 1 H), 4.20-4.32 (m, |
| | | 2 H), 3.12 (s, 6 H), 2.03-2.25 (m, 2 H) |
| 1.53 | Rₜ = 1.16; | ¹H NMR (400 MHz, DMSO-d6) δ [ppm]: 9.23 (d, J = 8 Hz, |
| | m/z (ES−) = | 1H), 8.77 (s, 1 H), 8.65 (d, J = 5.6 Hz, 1 H), 8.49 (s, 1 H), 8.25 |
| | 494.0 [M − H]⁻/ | (d, J = 5.6 Hz, 1 H), 8.25 (d, J = 5.6 Hz, 1 H), 8.09 (d, J = 2 Hz, |
| | Method B | 1 H), 8.07 (d, J = 5.6 Hz, 1 H), 7. 73 (t, J = 2 Hz, 1 H), 6.83 (d, |
| | | J = 5.6 Hz, 1 H), 5.28 (q, J = 7.2 Hz, 1 H), 4.29-4.43 (m, 2 H), |
| | | 3.10 (s, 6 H), 2.09-2.27 (m, 2 H) |
| 1.54 | Rₜ = 2.93; | ¹H NMR (400 MHz, DMSO-d6) δ [ppm]: 9.15 (d, J = 8.4 Hz, |
| | m/z (ES−) = | 1 H), 8.62 (d, J = 6 Hz, 1 H), 8.58 (s, 1 H), 8.09 (d, J = 5.6 Hz, |
| | 493.0 | 1 H), 7.73 (d, J = 2 Hz, 1 H), 7.53-7.55 (m, 2 H), 7.34 (d, J = |
| | [M + H]⁺/ | 6.8 Hz, 1 H), 7.16 (td, J = 1.6, 8.8 Hz, 1 H), 6.91 (td, J = 1.2, |
| | Method C | 7.6 Hz, 1 H), 3.81 (d, J = 7 Hz, 1 H), 5.22 (q, J = 5.6 Hz, 1 H), |
| | | 4.19-4.30 (m, 2 H), 3.11 (s, 6 H), 2.16-2.23 (m, 1 H), 2.01- |
| | | 2.07 (m, 1 H) |
| 1.55 | Rₜ = 2.89; | ¹H NMR (400 MHz, DMSO-d6) δ [ppm]: 9.15 (d, J = 8.4 Hz, |
| | m/z (ES−) = | 1 H), 8.62 (d, J = 6 Hz, 1 H), 8.59 (s, 1 H), 8.10 (d, J = 6 Hz, 1 |
| | 493.0 | H), 7.73 (dd, J = 1.6, 8 Hz, 1 H), 7.48 (t, J = 7.6 Hz, 1 H), 7.39- |
| | [M + H]⁺/ | 7.42 (m 1 H), 7.34 (d, J = 6.8 Hz, 1 H), 7.16 (td, J = 1.6, 8.4 Hz, |
| | Method C | 1 H), 6.90 (td, J = 0.8, 8.4 Hz, 1 H), 6.79 (d, J = 8 Hz, 1 H), |
| | | 5.22 (q, J = 5.6 Hz, 1 H), 4.21-4.27 (m, 2 H), 3.12 (s, 6 H), |
| | | 2.15-2.22 (m, 1 H), 2.00-2.06 (m, 1 H) |

The compounds of formula (I) of the present invention are useful for the treatment and/or control, in particular helminths, in which the endoparasitic nematodes and trematodes may be the cause of serious diseases of mammals and poultry. Typical nematodes of this indication are: Filariidae, Setariidae, Haemonchus, Trichostrongylus, Ostertagia, Nematodirus, Cooperia, Ascaris, Bunostonum, Oesophagostonum, Charbertia, Trichuris, Strongylus, Trichonema, Dictyocaulus, Capillaria, Heterakis, Toxocara, Ascaridia, Oxyuris, Ancylostoma, Uncinaria, Toxascaris and Parascaris. The trematodes include, in particular, the family of Fasciolideae, especially Fasciola hepatica.

Certain parasites of the species Nematodirus, Cooperia and Oesophagostonum infest

the intestinal tract of the host animal, while others of the species Haemonchus and

Ostertagia are parasitic in the stomach and those of the species Dictyocaulus are parasitic
in the lung tissue. Parasites of the families and may be found in the internal cell tissue and
in the organs, e.g. the heart, the blood vessels, the lymph vessels and the subcutaneous
tissue. A particularly notable parasite is the heartworm of the dog, Dirofilaria iminitis.

The parasites which may be treated and/or controlled by the compounds of formula (I)
also include those from the class of Cestoda (tapeworms), e.g. the families Mesocestoidae, especially of the genus Mesocestoides, in particular M. lineatus;
Dipylidiidae, especially Dipylidium caninum, Joyeuxiella spp., in particular Joyeuxiella
pasquali, and Diplopylidium spp., and Taeniidae, especially Taenia pisformis, Taenia
cervi, Taenia ovis, Taeneia hydatigena, Taenia multiceps, Taenia taeniaeformis, Taenia
serialis, and Echinococcus spp., most particularly Taneia hydatigena, Taenia ovis, Taenia
multiceps, Taenia serialis; Echinococcus granulosus and Echinococcus multilocularis.

Furthermore, the compounds of formula (I) are suitable for the treatment and/or control of human pathogenic parasites. Of these, typical representatives that appear in the digestive tract are those of the genus Ancylostoma, Necator, Ascaris, Strongyloides, Trichinella, Capillaria, Trichuris and Enterobius. The compounds of the present invention are also against parasites of the genus Wuchereria, Brugia, Onchocerca and Loa from the family
of Dracunculus and parasites of the genus Strongyloides and Trichinella, which infect
the gastrointestinal tract in particular.

A particular parasite to be treated and/or and controlled by the compounds of the invention is the heartworm (Dirofilaria immitis). Particular subjects for such treatment are dogs and cats.

The compounds of the invention can be administered alone or in the form of a composition. In practice, the compounds of the invention are usually administered in the form of compositions, that is, in admixture with at least one acceptable excipient. The proportion and nature of any acceptable excipient(s) are determined by the properties of the selected compound of the invention, the chosen route of administration, and standard practice as in the veterinary and pharmaceutical fields.

In one embodiment, the present invention provides compositions comprising: a compound of invention and at least one acceptable excipient.

In effecting such treatment and/or control, a compound of the invention can be administered in any form and route which makes the compound bioavailable. The compounds of the invention can be administered by a variety of routes, including orally, in particularly by tablets and capsules. The compounds of the invention can be administered parenteral routes, more particularly by inhalation, subcutaneously, intramuscularly, intravenously, intraarterially, transdermally, intranasally, rectally, vaginally, ocularly, topically, sublingually, and buccally, intraperitoneally, intraadiposally, intrathecally and via local delivery for example by catheter or stent.

One skilled in the art can readily select the proper form and route of administration depending upon the particular characteristics of the compound selected, the disorder or condition to be treated, the stage of the disorder or condition, and other relevant circumstances. The pharmaceutical compositions of the invention may be administered to the subject, for example, in the form of tablets, capsules, cachets, papers, lozenges, wafers, elixirs, ointments, transdermal patches, aerosols, inhalants, suppositories, drenches, solutions, and suspensions.

The term “acceptable excipient” refers to refers to those typically used in preparing veterinary and pharmaceutical compositions and should be pure and non-toxic in the amounts used. They generally are a solid, semi-solid, or liquid material which in the aggregate can serve as a vehicle or medium for the active ingredient. Some examples of acceptable excipients are found in Remington's Pharmaceutical Sciences and the Handbook of Pharmaceutical Excipients and include diluents, vehicles, carriers, ointment bases, binders, disintegrates, lubricants, glidants, sweetening agents, flavoring agents, gel bases, sustained release matrices, stabilizing agents, preservatives, solvents, suspending agents, buffers, emulsifiers, dyes, propellants, coating agents, and others.

In one embodiment, the composition is adapted for oral administration, such as a tablet or a capsule or a liquid formulation, for example, a solution or suspension, adapted for oral administration. In one embodiment, the composition is adapted for oral administration, such as chewable formulation, adapted for oral administration. In still another embodiment, the composition is a liquid or semi-solid formulation, for example, a solution or suspension or a paste, adapted for parenteral administration.

Particular compositions for usage on subjects in the treatment and/or control of nematodes/helminths comprise solutions; emulsions including classical emulsions, microemulsions and self-emulsifying compositions, that are waterless organic, preferably oily, compositions which form emulsions, together with body fluids, upon addition to the subject's body; suspensions (drenches); pour-on formulations; food additives; powders; tablets including effervescent tablets; boli; capsules including micro-capsules; and chewable treats. Particularly composition forms are tablets, capsules, food additives or chewable treats.

The compositions of the present invention are prepared in a manner well known in the veterinary and pharmaceutical art and include at least one of the compounds of the invention as the active ingredient. The amount of a compound of the present invention may be varied depending upon its particular form and may conveniently be between 1% to about 50% of the weight of the unit dose form. The present pharmaceutical compositions are preferably formulated in a unit dose form, each dose typically containing from about 0.5 mg to about 100 mg of a compounds of the invention. One or more unit dose form(s) may be taken to affect the treatment dosage.

In one embodiment, the present invention also provides a method for treating parasites, comprising: administering to a subject in need thereof an effective amount of a compound of formula (I) or a salt thereof, the method optionally further comprising an effective amount of at least one additional active compound.

In one embodiment, the present invention also provides a method for controlling parasites, comprising: administering to a subject in need thereof an effective amount of a compound of formula (I) or a salt thereof, the method optionally further comprising an effective amount of at least one additional active compound.

In one embodiment, the present invention also provides a method for treating or controlling parasites, comprising: contacting a subject's environment with an effective amount of a compound of formula (I) or a salt thereof, the method optionally further comprising an effective amount of at least one additional active compound.

Thus, the invention provides for the use of the compounds of the invention as a medicament, including for the manufacture of a medicament. In one embodiment, the invention provides the manufacture of a medicament comprising a compound of formula (I) or a salt thereof for treating parasites. In one embodiment, the invention provides the manufacture of a medicament comprising a compound of the invention or a salt thereof for controlling parasites.

The terms “treating”, “to treat”, “treated”, or “treatment”, include without limitation restraining, slowing, stopping, reducing, ameliorating, reversing the progression or severity of an existing symptom, or preventing a disorder, condition, or disease. For example, an adult heartworm infection would be treated by administering a compound of the invention. A treatment may be applied or administered therapeutically.

The terms “control”, “controlling” or “controlled” refers to include without limitation decreasing, reducing, or ameliorating the risk of a symptom, disorder, condition, or disease, and protecting an animal from a symptom, disorder, condition, or disease. Controlling may refer to therapeutic, prophylactic, or preventative administration. It is well understood that a larvae or immature heartworm infection may be asymptomatic and infection by mature parasites is symptomatic and/or debilitating, Therefore, for example, a heartworm infection would be controlled by acting on the larvae or immature parasite preventing the infection from progressing to an infection by mature parasites.

Thus, the use of the compounds of the invention in the treatment and/or control of parasites, in particular helminths, in which the endoparasitic nematodes and trematodes refers to the use of the compounds of the invention to act on the various forms of the parasites throughout its life cycle, independent of whether a subject is manifesting a symptom, including morbidity or mortality, and independently of the phase(s) of the parasitic challenge.

As used herein, “administering to a subject” includes but is not limited to cutaneous, subcutaneous, intramuscular, mucosal, submucosal, transdermal, oral or intranasal administration. Administration could include injection or topical administration.

The terms “subject” and “patient” refers includes humans and non-human mammalian animals, such as dogs, cats, mice, rats, guinea pigs, rabbits, ferrets, cows, horses, sheep, goats, and pigs. It is understood that a more particular subject is a human. Also, a more particular subject are mammalian pets or companion animals, such as dogs and cats and also mice, guinea pigs, ferrets, and rabbits.

The term “effective amount” refers to an amount which gives the desired benefit to the subject and includes administration for both treatment and control. The amount will vary from one individual subject to another and will depend upon a number of factors, including the overall physical condition of the subject and the severity of the underlying cause of the condition to be treated, concomitant treatments, and the amount of compound of the invention used to maintain desired response at a beneficial level.

An effective amount can be readily determined by the attending diagnostician, as one skilled in the art, by the use of known techniques and by observing results obtained under analogous circumstances. In determining the effective amount, the dose, a number of factors are considered by the attending diagnostician, including, but not limited to: the species of patient; its size, age, and general health; the specific condition, disorder, infection, or disease involved; the degree of or involvement or the severity of the condition, disorder, or disease, the response of the individual patient; the particular compound administered; the mode of administration; the bioavailability characteristics of the preparation administered; the dose regimen selected; the use of concomitant medication; and other relevant circumstances. An effective amount of the present invention, the treatment dosage, is expected to range from 0.5 mg to 100 mg. Specific amounts can be determined by the skilled person. Although these dosages are based on a subject having a mass of about 1 kg to about 20 kg, the diagnostician will be able to determine the appropriate dose for a subject whose mass falls outside of this weight range. An effective amount of the present invention, the treatment dosage, is expected to range from 0.1 mg to 10 mg/kg of the subject. The dosing regimen is expected to be daily, weekly, or monthly administration.

The compounds of the invention may be combined with one or more other active compounds or therapies for the treatment of one or more disorders, diseases or conditions, including the treatment of parasites, for which it is indicated. The compounds of the invention may be administered simultaneously, sequentially or separately in combination with one or more compounds or therapies for treating parasites and other disorders.

For example, when used to treat parasites, including heartworm, a compound of the invention may be combined with a macrocyclic lactone such as ivermectin, moxidectin, or milbemycin oxime, or with imidacloprid. Particular combinations for treating parasites include a compound of the invention and ivermectin. Another particular combination for treating parasites include a compound of the invention and milbemycin oxime.

Thus, it is understood that the compositions and methods of the present invention optionally include comprising an effective amount of at least one additional active compound.

The activity of compounds as parasiticides may be determined by a variety of methods, including in vitro and in vivo methods.

### Example A

### Dog Heart Worm Microfilariae

D. immitis microfilariae are isolated by filtration from beagle blood of an infected donor and allowed to incubate in appropriate media. Test compounds are diluted in DMSO and added to a 96-well plate containing parasites. Plates are incubated for the desired time and motility is assessed using an LCD camera imaging system. Effect of serum is tested by addition of up to 20% fetal bovine serum in the assay. Percent motility inhibition values are generated relative to the average of the DMSO-only wells.

In this test for example, the following compounds from the preparation examples showed EC50<0.1 μg/mL: 1.1, 1.8, 1.9, 1.12, 1.15, 1.17, 1.20, 1.21, 1.24, 1.30, 1.31, 1.32, 1.33, 1.37, 1.38, 1.39, 1.42, 1.45, 1.47, 1.48, 1.49, 1.50, 1.51, 1.52, 1.54, 1.55, 2.1, 4.1, 5.1, 6.1, and 7.1.

### Example B

Ruminant Gastrointestinal (H. contortus (H.c.)):

H.c. eggs isolated from lamb fecal matter are allowed to hatch overnight. Test compounds are diluted in DMSO and added to a 96-well plate containing appropriate media. H.c. larvae are added to each well and plates are incubated for the desired time(s). Motility is assessed using an LCD camera imaging system. Percent motility inhibition values are generated relative to the average of the DMSO-only wells.

In this test for example, the following compounds from the preparation examples showed EC50<1 μg/mL: 1.1, 1.7, 1.8, 1.9, 1.12, 1.17, 1.20, 1.21, 1.24, 1.25, 1.31, 1.32, 1.33, 1.37, 1.38, 1.39, 1.42, 1.45, 1.47, 1.48, 1.50, 1.51, 1.54, 1.55, 2.1, 3.1, 4.1, 5.1, 6.1, and 7.1.

### Example C

### Gastro Intestinal Nematodes

Jirds (Meriones unguiculatus), are artificially infected by gavage with third instar larvae each of T. colubriformis and H. contortus. Then treated orally with the test compound formulated in eg DMSO/PEG 2/1, on Day 6 after infection at a dose in a range between 1×3 mg/kg up to 1×32 mg/kg. Three days after treatment, gerbils are euthanized and dissected to recover H. contortus from stomach and T. colubriformis from the small intestine. Efficacy is expressed as a % reduction in worm numbers in comparison with a placebo treated group, using the Abbot's formula. Compound Nos. 1.1, 1.9, 1.20, 1.21, and 1.31 showed an efficacy >90% in this model. Compound No. 1.50 showed an efficacy >90% against He in this model.

### Example D

### Filarial Nematodes

Av model: Gerbils, injected subcutaneously with infective A. viteae larvae, were subsequently treated with the test article formulated in eg DMSO/PEG 2/1, by oral gavage at a dose in a range between 1×3 mg/kg up to 5×32 mg/kg (one dose per day for 5 consecutive days). At necropsy 12 weeks after infection, efficacy is expressed as a % reduction in worm numbers in comparison with the placebo treated group, using the Abbot's formula. Compound Nos. 1.1, 1.9, 1.20, 1.31, 1.32, 1.42 and 5.1 showed efficacy of >80% in this model.

### Example E

L.s. Model

Mice, injected subcutaneously with infective L. sigmodontis larvae, were subsequently treated with the test article formulated in eg DMSO/PEG 2/1, by oral gavage at a dose in a range between 1×3 mg/kg up to 5×32 mg/kg (one dose per day for 5 consecutive days). At necropsy 5 weeks after infection, efficacy is calculated by counting developed larvae vs. untreated animals using Abbot's formula. Compound 1.8 showed an efficacy of >70% in this model.

We claim:

1. A compound of formula (I): 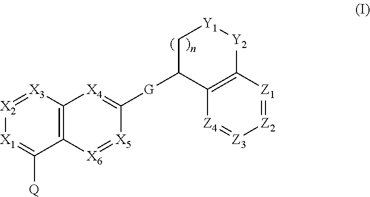
wherein
n is 0 or 1;
X₁ is selected from the group consisting of N and CR₁;
X₂ is selected from the group consisting of N and CR₂;
X₃ is selected from the group consisting of N and CR₃;
X₄ is selected from the group consisting of N and CR₄;
X₅ is selected from the group consisting of N and CR₅;
X₆ is selected from the group consisting of N and CR₆;
wherein at least one of X₁, X₂, X₃, and X₅ is N or wherein none of X₁, X₂, X₃, X₄, X₅, and X₆ are N;
G is the group 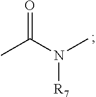
Y₁ is selected from the group consisting of CR₈R₉, O, S, and NR₁₀;
Y₂ is selected from the group consisting of CR₈R₉, O, S, and NR₁₀;
wherein at least one of the groups Y₁ or Y₂ is CR₈R₉;
Z₁ is selected from the group consisting of N, O, S, and CR₁₁;
Z₂ is selected from the group consisting of nil, N, and CR₁₁;
Z₃ is selected from the group consisting of nil, N and CR₁₁;
Z₄ is selected from the group consisting of N, O, S, and CR₁₁;
wherein no more than 2 of Z₁, Z₂, Z₃, and Z₄ are N and wherein only one of Z₁ and Z₄ is O or S, Z₂ is nil only when Z₁ is O or S, and Z₃ is nil only when Z₄ is O or S;
R₁ is selected from the group consisting of hydrogen, halogen, hydroxyl, —SH, —SC₁-C₄ alkyl, —S(O)(C₁-C₄ alkyl, —S(O)₂(C₁-C₄ alkyl, cyano, C₁-C₄ alkyl, C₁-C₄ halogenoalkyl, C₁-C₄-alkoxy, —B(OR₁₂)(OR₁₃) wherein R₁₂ is, each time taken, selected from the group consisting or hydrogen, C₁-C₄ alkyl, and C₃-C₆ cycloalkyl, R₁₃ is, each time taken, selected from the group consisting or hydrogen, C₁-C₄ alkyl, and C₃-C₆ cycloalkyl, or R₁₂ and R₁₃ together with the oxygen atoms to which they are attached form a 5- to 7-membered ring which is optionally substituted with 1 to 4 C₁-C₄ alkyl; —NH₂, —NH(C₁-C₄ alkyl), and —N(C₁-C₄ alkyl)₂;
R₂ is selected from the group consisting of hydrogen, halogen, hydroxyl, —SH, —SC₁-C₄ alkyl, —S(O)(C₁-C₄ alkyl, —S(O)₂(C₁-C₄ alkyl, cyano, C₁-C₄ alkyl, C₁-C₄ halogenoalkyl, C₁-C₄-alkoxy, —B(OR₁₂)(OR₁₃) wherein R₁₂ is, each time taken, selected from the group consisting or hydrogen, C₁-C₄ alkyl, and C₃-C₆ cycloalkyl, R₁₃ is, each time taken, selected from the group consisting or hydrogen, C₁-C₄ alkyl, and C₃-C₆ cycloalkyl, or R₁₂ and R₁₃ together with the oxygen atoms to which they are attached form a 5- to 7-membered ring which is optionally substituted with 1 to 4 C₁-C₄ alkyl; —NH₂, —NH(C₁-C₄ alkyl), and —N(C₁-C₄ alkyl)₂;
R₃ is selected from the group consisting of hydrogen, halogen, hydroxyl, —SH, —SC₁-C₄ alkyl, —S(O)(C₁-C₄ alkyl, —S(O)₂(C₁-C₄ alkyl, cyano, C₁-C₄ alkyl, C₁-C₄ halogenoalkyl, C₁-C₄-alkoxy, —B(OR₁₂)(OR₁₃) wherein R₁₂ is, each time taken, selected from the group consisting or hydrogen, C₁-C₄ alkyl, and C₃-C₆ cycloalkyl, R₁₃ is, each time taken, selected from the group consisting or hydrogen, C₁-C₄ alkyl, and C₃-C₆ cycloalkyl, or R₁₂ and R₁₃ together with the oxygen atoms to which they are attached form a 5- to 7-membered ring which is optionally substituted with 1 to 4 C₁-C₄ alkyl; —NH₂, —NH(C₁-C₄ alkyl), and —N(C₁-C₄alkyl)₂;
R₄ is selected from the group consisting of halogen, cyano, —CHO, hydroxyl, C₁-C₄alkyl, C₂-C₄ alkenyl, C₂-C₄ alkynyl, C₃-C₆ cycloalkyl, C₁-C₄ halogenoalkyl, C₁-C₄-alkoxy substituted-C₁-C₄ alkyl, benzyl optionally substituted with 1 to 5 halogen atoms, C₁-C₄ alkoxy, —NH₂, —NH(C₁-C₄ alkyl), —N(C₁-C₄ alkyl)₂, —NH(C₃-C₆cycloalkyl), —N(C₁-C₄ alkyl)(C₃-C₆-cycloalkyl), —NH(4- to 7-membered heterocycloalkyl), —N(C₁-C₄ alkyl)(4- to 7-membered heterocycloalkyl), —N(C₁-C₄ alkyl)(C₃-C₆-cycloalkyl), —N(C₁-C₄ alkyl)(C₁-C₄ alkoxy), —C(O)NH(C₁-C₄ alkyl), —C(O)N(C₁-C₄ alkyl)₂, —NHSO₂(C₁-C₄ alkyl), —SC₁-C₄ alkyl, —S(O)C₁-C₄ alkyl, —SO₂C₁-C₄ alkyl, —B(OR₁₂)(OR₁₃) wherein R₁₂ is, each time taken, selected from the group consisting or hydrogen, C₁-C₄ alkyl, and C₃-C₆ cycloalkyl, R₁₃ is, each time taken, selected from the group consisting or hydrogen, C₁-C₄ alkyl, and C₃-C₆cycloalkyl, or R₁₂ and R₁₃ together with the oxygen atoms to which they are attached form a 5- to 7-membered ring which is optionally substituted with 1 to 4 C₁-C₄ alkyl; 6- or 10 membered aryl; and a monocyclic heterocycle selected from the group of 4- to 7-membered heterocycloalkyl, 5-membered heteroaryl having at least one nitrogen atom via which the 5-membered heteroaryl ring is connected to the rest of the molecule; 6-membered heteroaryl having at least one nitrogen atom; each of the aryl, heterocycloalkyl, and heteroaryl ring in R₄ is optionally substituted with 1, 2 or 3 substituents independently selected from the group consisting of halogen, cyano, nitro, hydroxyl, oxo, C₁-C₄ alkyl, C₃-C₆ cycloalkyl, C₁-C₄ halogenoalkyl, C₁-C₄ alkoxy, —NH₂, —NH(C₁-C₄ alkyl), —N(C₁-C₄ alkyl)₂, —NH(C₃-C₆ cycloalkyl), —N(C₁-C₄ alkyl)(C₃-C₆-cycloalkyl), —NHSO₂(C₁-C₄ alkyl), —SC₁-C₄ alkyl, —S(O)C₁-C₄ alkyl, —SO₂C₁-C₄ alkyl, —S(O)C₁-C₄-halogenoalkyl and —SO₂C₁-C₄ halogenoalkyl; and wherein each C₁-C₄ alkyl, C₃-C₆ cycloalkyl and C₁-C₄ alkoxy in R₄ may be optionally substituted with 1, 2 or 3 substituents independently selected from the group consisting of halogen, hydroxyl, —NH₂, —NH(C₁-C₄ alkyl), —N(C₁-C₄ alkyl)₂, cyano, carboxyl, carbamoyl, C₁-C₄ alkoxycarbonyl, —C(O)NH(C₁-C₄ alkyl), —C(O)N(C₁-C₄ alkyl)₂, and C₁-C₄ alkoxy;
R₅ is selected from the group consisting of hydrogen, halogen, hydroxyl, —SH, —SC₁-C₄ alkyl, —S(O)(C₁-C₄ alkyl, —S(O)₂(C₁-C₄ alkyl, cyano, C₁-C₄ alkyl, C₁-C₄ halogenoalkyl, C₁-C₄-alkoxy, —B(OR₁₂)(OR₁₃) wherein R₁₂ is, each time taken, selected from the group consisting or hydrogen, C₁-C₄ alkyl, and C₃-C₆ cycloalkyl, R₁₃ is, each time taken, selected from the group consisting or hydrogen, C₁-C₄ alkyl, and C₃-C₆ cycloalkyl, or R₁₂ and R₁₃ together with the oxygen atoms to which they are attached form a 5- to 7-membered ring which is optionally substituted with 1 to 4 C₁-C₄ alkyl; —NH₂, —NH(C₁-C₄ alkyl), and —N(C₁-C₄ alkyl)₂;
R₆ is selected from the group consisting of hydrogen, halogen, hydroxyl, —SH, —SC₁-C₄ alkyl, —S(O)(C₁-C₄ alkyl, —S(O)₂(C₁-C₄ alkyl, cyano, C₁-C₄ alkyl, C₁-C₄ halogenoalkyl, C₁-C₄-alkoxy, —B(OR₁₂)(OR₁₃) wherein R₁₂ is, each time taken, selected from the group consisting or hydrogen, C₁-C₄ alkyl, and C₃-C₆ cycloalkyl, R₁₃ is, each time taken, selected from the group consisting or hydrogen, C₁-C₄ alkyl, and C₃-C₆ cycloalkyl, or R₁₂ and R₁₃ together with the oxygen atoms to which they are attached form a 5- to 7-membered ring which is optionally substituted with 1 to 4 C₁-C₄ alkyl; —NH₂, —NH(C₁-C₄ alkyl), and —N(C₁-C₄ alkyl)₂;
R₇ is selected from the group consisting of hydrogen, C₁-C₄ alkyl, and C₃-C₆ cycloalkyl optionally substituted with 1 to 5 halogen atoms, —C(H)O, C₂-C₄ alkenyl, C₂-C₄ alkynyl, C₁-C₄ halogenoalkyl, and C₁-C₄-alkoxy;
R₈ is, each time selected, independently selected from the group consisting of hydrogen, fluoro, and C₁-C₄ alkyl;
R₉ is, each time selected, independently selected from the group consisting of hydrogen, fluoro, and C₁-C₄ alkyl;
R₁₀ is selected from the group consisting of hydrogen and C₁-C₄ alkyl;
R₁₁ is, each time selected, independently selected from the group consisting of hydrogen, halogen, hydroxyl, cyano, C₁-C₄ alkyl, C₁-C₄ halogenoalkyl, C₁-C₄-alkoxy, C₃-C₆ cycloalkyl, —NH₂, —NH(C₁-C₄ alkyl), and —N(C₁-C₄ alkyl)₂; and
Q is selected from the group consisting of 6- or 10 membered aryl optionally substituted with 1, 2 or 3 substituents independently selected from the group consisting of halogen, cyano, nitro, hydroxyl, C₁-C₄ alkyl, C₁-C₄ halogenoalkyl, C₁-C₄ alkoxy, C₃-C₆ cycloalkyl,
—NH₂, —NH(C₁-C₄ alkyl), —N(C₁-C₄ alkyl)₂, —NH(C₃-C₆ cycloalkyl), —N(C₁-C₄ alkyl)(C₃-C₆-cycloalkyl), —NHSO₂(C₁-C₄ alkyl), —SC₁-C₄ alkyl, —S(O)C₁-C₄ alkyl, —SO₂C₁-C₄ alkyl,
—S(O)C₁-C₄-halogenoalkyl and —SO₂C₁-C₄ halogenoalkyl, wherein the 6- or 10 membered aryl is optionally fused with a 4- to 7-membered heterocycloalkyl having 1 or 2 heteroatoms selected from the group O, S, and N and wherein the carbons of the heterocycloalkyl are optionally substituted with 1, 2 or 3 substituents independently selected from the group halogen, cyano, nitro, hydroxyl, oxo, C₁-C₄ alkyl, C₃-C₆ cycloalkyl, C₁-C₄ halogenoalkyl, C₁-C₄ alkoxy, —NH₂, —NH(C₁-C₄ alkyl), and —N(C₁-C₄ alkyl)₂ and any N in the heterocycloalkyl is, valency permitting, substituted with a substituent selected from the group consisting of hydrogen, C₁-C₄ alkyl, and C₃-C₆ cycloalkyl; 5- to 10-membered heteroaryl having 1 or 2 heteroatoms selected from the group O, S, and N and wherein the carbons of the 5- to 10-membered heteroaryl are optionally substituted with 1, 2 or 3 substituents independently selected from the group consisting of halogen, cyano, nitro, hydroxyl, C₁-C₄ alkyl, C₃-C₆ cycloalkyl, C₁-C₄ halogenoalkyl, C₁-C₄ alkoxy, —NH₂, —NH(C₁-C₄ alkyl), and —N(C₁-C₄ alkyl)₂ and any N in the heteroaryl, valency permitting, is optionally substituted with a substituent selected from the group consisting of hydrogen, C₁-C₄ alkyl, and C₃-C₆ cycloalkyl; a 4- to 7-membered heterocycloalkyl having 1 or 2 heteroatoms selected from the group O, S, N, wherein the heterocycloalkyl is optionally benzo-fused, wherein the carbons of the 4- to 7-membered heterocycloalkyl or optionally benzo-fused 4- to 7-membered heterocycloalkyl are optionally substituted with 1, 2, 3, or 4 substituents independently selected from the group consisting of halogen, cyano, nitro, hydroxyl, oxo, C₁-C₄ alkyl, C₃-C₆ cycloalkyl, C₁-C₄ halogenoalkyl, C₁-C₄ alkoxy, —NH₂, —NH(C₁-C₄ alkyl), and —N(C₁-C₄ alkyl)₂ and any N in the heterocycloalkyl is optionally substituted with a substituent selected from the group consisting of hydrogen, C₁-C₄ alkyl, and C₃-C₆ cycloalkyl; 6- or 10 membered aryloxy optionally substituted with 1, 2 or 3 substituents independently selected from the group consisting of halogen, cyano, nitro, hydroxyl, C₁-C₄ alkyl, C₃-C₆ cycloalkyl, C₁-C₄ halogenoalkyl, C₁-C₄ alkoxy, —NH₂, —NH(C₁-C₄ alkyl), —N(C₁-C₄ alkyl)₂, —NH(C₃-C₆ cycloalkyl), —N(C₁-C₄ alkyl)(C₃-C₆-cycloalkyl), —NHSO₂(C₁-C₄ alkyl), —SC₁-C₄ alkyl, —S(O)C₁-C₄ alkyl, —SO₂C₁-C₄ alkyl, —S(O)C₁-C₄-halogenoalkyl and —SO₂C₁-C₄ halogenoalkyl; 6- or 10 membered arylthio-oxy optionally substituted with 1, 2 or 3 substituents independently selected from the group consisting of halogen, cyano, nitro, hydroxyl, C₁-C₄ alkyl, C₃-C₆ cycloalkyl, C₁-C₄ halogenoalkyl, C₁-C₄ alkoxy, —NH₂, —NH(C₁-C₄ alkyl), —N(C₁-C₄ alkyl)₂, —NH(C₃-C₆ cycloalkyl), —N(C₁-C₄ alkyl)(C₃-C₆-cycloalkyl), —NHSO₂(C₁-C₄ alkyl), —SC₁-C₄ alkyl, —S(O)C₁-C₄ alkyl, —SO₂C₁-C₄ alkyl, —S(O)C₁-C₄-halogenoalkyl and —SO₂C₁-C₄ halogenoalkyl; and 5- to 10-membered heteroaryloxy optionally substituted with 1, 2 or 3 substituents independently selected from the group consisting of halogen, cyano, nitro, hydroxyl, oxo, C₁-C₄ alkyl, C₃-C₆ cycloalkyl, C₁-C₄ halogenoalkyl, C₁-C₄ alkoxy, —NH₂, —NH(C₁-C₄ alkyl), —N(C₁-C₄ alkyl)₂, —NH(C₃-C₆ cycloalkyl), —N(C₁-C₄ alkyl)(C₃-C₆-cycloalkyl), —NHSO₂(C₁-C₄ alkyl), —SC₁-C₄ alkyl, —S(O)C₁-C₄ alkyl, —SO₂C₁-C₄ alkyl, —S(O)C₁-C₄-halogenoalkyl and
—SO₂C₁-C₄ halogenoalkyl;
excluding the compounds:
N-[(4S)-chroman-4-yl]-8-(3,5-dichlorophenyl)-4-(dimethylamino)-1,7-naphthyridine-3-carboxamide;
N-[(4S)-chroman-4-yl]-8-(2,3-dichlorophenyl)-4-(dimethylamino)-1,7-naphthyridine-3-carboxamide;
N-[(4S)-chroman-4-yl]-8-(3,5-dichlorophenyl)-4-(morpholin-4-yl)-1,7-naphthyridine-3-carboxamide;
N-[(4S)-chroman-4-yl]-8-(2,3-dichlorophenyl)-4-(morpholin-4-yl)-1,7-naphthyridine-3-carboxamide;
4-chloro-N-[(4S)-chroman-4-yl]-8-(2,3-dichlorophenyl)-1,6-naphthyridine-3-carboxamide;
8-bromo-N-[(4S)-chroman-4-yl]-4-oxo-1,4-dihydro-1,6-naphthyridine-3-carboxamide;
N-[(4S)-chroman-4-yl]-8-(3,5-dichlorophenyl)-4-oxo-1,4-dihydro-1,6-naphthyridine-3-carboxamide;
N-[(4S)-chroman-4-yl]-8-(2,3-dichlorophenyl)-4-oxo-1,4-dihydro-1,6-naphthyridine-3-carboxamide;
4-chloro-N-[(4S)-chroman-4-yl]-8-(3,5-dichlorophenyl)-1,6-naphthyridine-3-carboxamide;
4-chloro-N-[(4S)-chroman-4-yl]-8-(2,3-dichlorophenyl)-1,6-naphthyridine-3-carboxamide;
N-[(4S)-chroman-4-yl]-8-(3,5-dichlorophenyl)-4-(dimethylamino)-1,6-naphthyridine-3-carboxamide;
N-[(4S)-chroman-4-yl]-8-(2,3-dichlorophenyl)-4-(dimethylamino)-1,6-naphthyridine-3-carboxamide;
N-[(4S)-chroman-4-yl]-8-(2,3-dichlorophenyl)-4-(morpholin-4-yl)-1,6-naphthyridine-3-carboxamide;
N-[(4S)-chroman-4-yl]-8-(3,5-dichlorophenyl)-4-(morpholin-4-yl)-1,6-naphthyridine-3-carboxamide;
4-chloro-N-[(4S)-chroman-4-yl]-8-(3,5-dichlorophenyl)-1,5-naphthyridine-3-carboxamide;
4-chloro-N-[(4S)-chroman-4-yl]-8-(2,3-dichlorophenyl)-1,5-naphthyridine-3-carboxamide;
N-[(4S)-chroman-4-yl]-8-(3,5-dichlorophenyl)-4-(dimethylamino)-1,5-naphthyridine-3-carboxamide;
N-[(4S)-chroman-4-yl]-8-(3,5-dichlorophenyl)-4-(morpholin-4-yl)-1,5-naphthyridine-3-carboxamide;
N-[(4S)-chroman-4-yl]-8-(2,3-dichlorophenyl)-4-(dimethylamino)-1,5-naphthyridine-3-carboxamide;
N-[(4S)-chroman-4-yl]-8-(2,3-dichlorophenyl)-4-(morpholin-4-yl)-1,5-naphthyridine-3-carboxamide;
8-chloro-4-(3,5-dichlorophenyl)-N-[(4S)-chroman-4-yl]pyrido[3,2-d]pyrimidine-7-carboxamide;
4-(3,5-dichlorophenyl)-N-[(4S)-chroman-4-yl]-8-(dimethylamino)-pyrido[3,2-d]pyrimidine-7-carboxamide;
4-(3,5-dichlorophenyl)-N-[(4S)-chroman-4-yl]-8-(morpholin-4-yl)-pyrido[3,2-d]pyrimidine-7-carboxamide;
8-bromo-4-chloro-4-N-[(4S)-chroman-4-yl]cinnoline-3-carboxamide;
8-bromo-4-N-[(4S)-chroman-4-yl]-4-(dimethylamino)cinnoline-3-carboxamide;
8-bromo-4-N-[(4S)-chroman-4-yl]-4-methoxycinnoline-3-carboxamide;
8-(3,5-dichlorophenyl)-4-N-[(4S)-chroman-4-yl]-4-(dimethylamino)cinnoline-3-carboxamide;
8-(3-chlorophenyl)-4-N-[(4S)-chroman-4-yl]-4-(dimethylamino)cinnoline-3-carboxamide;
8-(2,3-dichlorophenyl)-4-N-[(4S)-chroman-4-yl]-4-(dimethylamino)cinnoline-3-carboxamide;
8-(3,4-difluorophenyl)-4-N-[(4S)-chroman-4-yl]-4-(dimethylamino)cinnoline-3-carboxamide;
8-(3,5-dichlorophenyl)-4-N-[(4S)-chroman-4-yl]-4-methoxycinnoline-3-carboxamide;
8-(3-chlorophenyl)-4-N-[(4S)-chroman-4-yl]-4-methoxycinnoline-3-carboxamide;
8-(2,3-dichlorophenyl)-4-N-[(4S)-chroman-4-yl]-4-methoxycinnoline-3-carboxamide;
8-(3,4-fluorophenyl)-4-N-[(4S)-chroman-4-yl]-4-methoxycinnoline-3-carboxamide;
8-chloro-4-(2,3-dichlorophenyl)-N-[(4S)-chroman-4-yl]pyrido[3,2-d]pyridazine-7-carboxamide;
4-(2,3-dichlorophenyl)-N-[(4S)-chroman-4-yl]-8-(dimethylamino)-pyrido[3,2-d]pyridazine-7-carboxamide;
4-(2,3-dichlorophenyl)-N-[(4S)-chroman-4-yl]-8-(morpholin-4-yl)-pyrido[3,2-d]pyridazine-7-carboxamide;
4-(3,5-dichlorophenyl)-N-[(4S)-chroman-4-yl]-8-(dimethylamino)-pyrido[3,2-d]pyridazine-7-carboxamide;
4-(3,5-dichlorophenyl)-N-[(4S)-chroman-4-yl]-8-(morpholin-4-yl)-pyrido[3,2-d]pyridazine-7-carboxamide;
N-[(4S)-chroman-4-yl]-8-(2,3,5-trifluorophenyl)-4-(morpholin-4-yl)-1,7-naphthyridine-3-carboxamide;
N-[(4S)-chroman-4-yl]-8-(2,3,5-trichlorophenyl)-4-(morpholin-4-yl)-1,7-naphthyridine-3-carboxamide;
N-[(4S)-chroman-4-yl]-8-(2,3-dichlorophenyl)-5-fluoro-4-(morpholin-4-yl)-1,7-naphthyridine-3-carboxamide;
N-[(4S)-chroman-4-yl]-8-(2,3,5-trifluorophenyl)-5-fluoro-4-(morpholin-4-yl)-1,7-naphthyridine-3-carboxamide;
N-[(4S)-chroman-4-yl]-8-(2,3,5-trifluorophenyl)-5-methoxy-4-(morpholin-4-yl)-1,7-naphthyridine-3-carboxamide;
N-[(4S)-chroman-4-yl]-8-(2,3-dichlorophenyl)-5-fluoro-4-(dimethylamino)-1,7-naphthyridine-3-carboxamide;
N-[(4S)-chroman-4-yl]-8-(2,3-dichlorophenyl)-5-methoxy-4-(dimethylamino)-1,7-naphthyridine-3-carboxamide;
N-[(4S)-chroman-4-yl]-8-(2,3,5-trifluorophenyl)-5-fluoro-4-(dimethylamino)-1,7-naphthyridine-3-carboxamide;
N-[(4S)-chroman-4-yl]-8-(2,3,5-trifluorophenyl)-5-methoxy-4-(dimethylamino)-1,7-naphthyridine-3-carboxamide;
N-[(4S)-chroman-4-yl]-8-(2,3-dichlorophenyl)-5-methoxy-4-(morpholin-4-yl)-1,7-naphthyridine-3-carboxamide;
N-[(4S)-chroman-4-yl]-8-(3,5-dichlorophenyl)-5-methoxy-4-(dimethylamino)-1,6-naphthyridine-3-carboxamide;
N-[(4S)-chroman-4-yl]-8-(2,3-dichlorophenyl)-7-methoxy-4-(morpholin-4-yl)-1,6-naphthyridine-3-carboxamide;
N-[(4S)-chroman-4-yl]-8-(2,3-dichlorophenyl)-7-methoxy-4-(dimethylamino)-1,6-naphthyridine-3-carboxamide;
N-[(4S)-chroman-4-yl]-8-(3,5-dichlorophenyl)-7-methoxy-4-(dimethylamino)-1,6-naphthyridine-3-carboxamide;
N-[(4S)-chroman-4-yl]-8-(3,5-dichlorophenyl)-7-methoxy-4-(morpholin-4-yl)-1,6-naphthyridine-3-carboxamide;
N-[(4S)-chroman-4-yl]-8-(3,5-dichlorophenyl)-5-methoxy-4-(morpholin-4-yl)-1,6-naphthyridine-3-carboxamide;
N-[(4S)-chroman-4-yl]-8-(2,3-dichlorophenyl)-5-methoxy-4-(morpholin-4-yl)-1,6-naphthyridine-3-carboxamide;
N-[(4S)-chroman-4-yl]-8-(2,3-dichlorophenyl)-5-methoxy-4-(dimethylamino)-1,6-naphthyridine-3-carboxamide;
7-chloro-N-[(4S)-chroman-4-yl]-8-(2,3,5-trifluorophenyl)-4-(dimethylamino)-1,5-naphthyridine-3-carboxamide;
7-chloro-N-[(4S)-chroman-4-yl]-8-(2,3,5-trifluorophenyl)-4-(morpholin-4-yl)-1,5-naphthyridine-3-carboxamide;
7-chloro-N-[(4S)-chroman-4-yl]-8-(3,5-dichlorophenyl)-4-(dimethylamino)-1,5-naphthyridine-3-carboxamide;
7-chloro-N-[(4S)-chroman-4-yl]-8-(2,3-dichlorophenyl)-4-(dimethylamino)-1,5-naphthyridine-3-carboxamide;
7-chloro-N-[(4S)-chroman-4-yl]-8-(2,3-dichlorophenyl)-4-(morpholin-4-yl)-1,5-naphthyridine-3-carboxamide;
7-methoxy-N-[(4S)-chroman-4-yl]-8-(3,5-dichlorophenyl)-4-(dimethylamino)-1,5-naphthyridine-3-carboxamide;
7-fluoro-N-[(4S)-chroman-4-yl]-8-(3,5-dichlorophenyl)-4-(dimethylamino)-1,5-naphthyridine-3-carboxamide;
7-methoxy-N-[(4S)-chroman-4-yl]-8-(2,3-dichlorophenyl)-4-(dimethylamino)-1,5-naphthyridine-3-carboxamide;
7-methoxy-N-[(4S)-chroman-4-yl]-8-(2,3-dichlorophenyl)-4-(morpholin-4-yl)-1,5-naphthyridine-3-carboxamide;
7-methoxy-N-[(4S)-chroman-4-yl]-8-(2,3,5-trifluorophenyl)-4-(dimethylamino)-1,5-naphthyridine-3-carboxamide;
7-methoxy-N-[(4S)-chroman-4-yl]-8-(2,3,5-trifluorophenyl)-4-(morpholin-4-yl)-1,5-naphthyridine-3-carboxamide;
7-fluoro-N-[(4S)-chroman-4-yl]-8-(2,3-dichlorophenyl)-4-(dimethylamino)-1,5-naphthyridine-3-carboxamide;
7-fluoro-N-[(4S)-chroman-4-yl]-8-(2,3-dichlorophenyl)-4-(morpholin-4-yl)-1,5-naphthyridine-3-carboxamide;
7-fluoro-N-[(4S)-chroman-4-yl]-8-(2,3,5-trifluorophenyl)-4-(dimethylamino)-1,5-naphthyridine-3-carboxamide; and
7-fluoro-N-[(4S)-chroman-4-yl]-8-(2,3,5-trifluorophenyl)-4-(morpholin-4-yl)-1,5-naphthyridine-3-carboxamide; or a salt thereof.

2. The compound according to claim 1, wherein X₁ is CR₁; X₂ is CR₂; X₃ is CR₃; X₄ is CR₄; X₅ is CR₅; and X₆ is CR₆; or a salt thereof.

3. The compound according to claim 1, wherein X₁ is N; X₂ is CR₂; X₃ is CR₃; X₄ is CR₄; X₅ is CR₅; and X₆ is N; or a salt thereof.

4. The compound according to claim 1, wherein X₁ is CR₁; X₂ is CR₂; X₃ is CR₃; X₄ is CR₄; X₅ is N; and X₆ is N; or a salt thereof.

5. The compound according to claim 1, wherein X₁ is CR₁; X₂ is CR₂; X₃ is CR₃; X₄ is CR₄; X₅ is N; and X₆ is CR₆; or a salt thereof.

6. The compound according to claim 1, wherein
Q is a 6- or 10 membered aryl optionally substituted with 1, 2 or 3 substituents independently selected from the group consisting of halogen, cyano, nitro, hydroxy, C₁-C₄ alkyl, C₁-C₄ halogenoalkyl, C₁-C₄ alkoxy, C₃-C₆ cycloalkyl, —NH₂, —NH(C₁-C₄ alkyl), —N(C₁-C₄ alkyl)₂, —NH(C₃-C₆ cycloalkyl), —N(C₁-C₄ alkyl)(C₃-C₆-cycloalkyl), —NHSO₂(C₁-C₄ alkyl), —SC₁-C₄ alkyl, —S(O)C₁-C₄ alkyl, —SO₂C₁-C₄ alkyl, —S(O)C₁-C₄-halogenoalkyl and —SO₂C₁-C₄ halogenoalkyl;
or a salt thereof.

7. The compound according to claim 1, wherein
Q is 6-membered aryl optionally substituted with 1, 2 or 3 substituents independently selected from the group consisting of halogen, cyano, nitro, hydroxy, C₁-C₄ alkyl, C₁-C₄ halogenoalkyl, C₁-C₄ alkoxy, C₃-C₆ cycloalkyl, —NH₂, —NH(C₁-C₄ alkyl), —N(C₁-C₄ alkyl)₂, —NH(C₃-C₆ cycloalkyl), —N(C₁-C₄ alkyl)(C₃-C₆-cycloalkyl), —NHSO₂(C₁-C₄ alkyl), —SC₁-C₄ alkyl, —S(O)C₁-C₄ alkyl, —SO₂C₁-C₄ alkyl, —S(O)C₁-C₄-halogenoalkyl and —SO₂C₁-C₄ halogenoalkyl, wherein the 6-membered aryl is fused with a 4- to 7-membered heterocycloalkyl having 1 or 2 heteroatoms selected from the group O, S, and N and wherein the carbons of the heterocycloalkyl are optionally substituted with 1, 2 or 3 substituents independently selected from the group consisting of halogen, cyano, nitro, hydroxy, oxo, C₁-C₄ alkyl, C₃-C₆ cycloalkyl, C₁-C₄ halogenoalkyl, C₁-C₄ alkoxy, —NH₂, —NH(C₁-C₄ alkyl), and —N(C₁-C₄ alkyl)₂ and any N in the heterocyclalkyl is substituted with a substituent selected from the group consisting of hydrogen, C₁-C₄ alkyl, and C₃-C₆ cycloalkyl; or a salt thereof.

8. The compound according to claim 1, wherein
Q is a 5- to 10-membered heteroaryl having 1 or 2 heteroatoms selected from the group O, S, and N and wherein the carbons of the heteroaryl are optionally substituted with 1, 2 or 3 substituents independently selected from the group consisting of halogen, cyano, nitro, —OH, C₁-C₄ alkyl, C₃-C₆ cycloalkyl, C₁-C₄ halogenoalkyl, C₁-C₄ alkoxy, —NH₂, —NH(C₁-C₄ alkyl), and —N(C₁-C₄ alkyl)₁ and any N in the heteroaryl is optionally substituted with a substituent selected from the group consisting of hydrogen, C₁-C₄ alkyl, and C₃-C₆ cycloalkyl;
or a salt thereof.

9. The compound according to claim 1, wherein
Q is a 4- to 7-membered heterocycloalkyl having 1 or 2 heteroatoms selected from the group O, S, N, wherein the heterocycloalkyl is optionally benzo-fused, wherein the carbons of the heterocycloalkyl or optionally benzo-fused heterocycloalkyl are optionally substituted with 1, 2, 3, or 4 substituents independently selected from the group consisting of halogen, cyano, nitro, hydroxy, oxo, C₁-C₄ alkyl, C₃-C₆ cycloalkyl, C₁-C₄ halogenoalkyl, C₁-C₄ alkoxy, —NH₂, —NH(C₁-C₄ alkyl), and —N(C₁-C₄ alkyl)₂ and any N in the heterocyclalkyl is optionally substituted with a substituent selected from the group consisting of hydrogen, C₁-C₄ alkyl, and C₃-C₆ cycloalkyl;
or a salt thereof.

10. The compound according to claim 1, wherein n is 1; or a salt thereof.

11. The compound according to claim 1, wherein Y₁ is CR₈R₉ and Y₂ is O; or a salt thereof.

12. The compound according to claim 1, wherein R₄ is selected from the group consisting of C₁-C₄ alkyl, C₃-C₆ cycloalkyl, —N(C₁-C₄ alkyl)₂, and 4- to 7-membered heterocycloalkyl; or a salt thereof.

13. The compound according to claim 1, selected from the group consisting of:
N-[(4S)-chroman-4-yl]-4-(dimethylamino)-8-[(1R)-tetralin-1-yl]-1,7-naphthyridine-3-carboxamide;
N-[(4S)-chroman-4-yl]-4-(dimethylamino)-8-[(1S)-tetralin-1-yl]-1,7-naphthyridine-3-carboxamide;
N-[(4S)-chroman-4-yl]-4-(dimethylamino)-8-[(1R or S)-tetralin-1-yl]-1,7-naphthyridine-3-carboxamide;
N-[(4S)-chroman-4-yl]-8-(1,2-dihydronaphthalen-1-yl)-4-(dimethylamino)-1,7-naphthyridine-3-carboxamide;
trans-8-(2,3,3a,4,5,6,7,7a-octahydroindol-1-yl)-N-[(4S)-chroman-4-yl]-4-(dimethylamino)-1,7-naphthyridine-3-carboxamide;
cis-8-(2,3,3a,4,5,6,7,7a-octahydroindol-1-yl)-N-[(4S)-chroman-4-yl]-4-(dimethylamino)-1,7-naphthyridine-3-carboxamide;
8-(5-chloroindolin-1-yl)-N-[(4S)-chroman-4-yl]-4-(dimethylamino)-1,7-naphthyridine-3-carboxamide;
N-(5-chloroindol-1-yl)-N-[(4S)-chroman-4-yl]-4-(dimethylamino)-1,7-naphthyridine-3-carboxamide;
N-[(4S)-chroman-4-yl]-4-morpholino-8-phenylsulfanyl-1,7-naphthyridine-3-carboxamide;
8-(benzenesulfonyl)-N-[(4S)-chroman-4-yl]-4-morpholino-1,7-naphthyridine-3-carboxamide;
4-amino-N-[(4S)-chroman-4-yl]-8-(3,5-dichlorophenyl)-1,7-naphthyridine-3-carboxamide;
N-[(4S)-chroman-4-yl]-8-(3,5-dichlorophenyl)-4-isopropoxy-1,7-naphthyridine-3-carboxamide;
N-[(4S)-chroman-4-yl]-8-(3,5-dichlorophenyl)-4-(4-methylpiperazin-1-yl)-1,7-naphthyridine-3-carboxamide;
N-[(4S)-chroman-4-yl]-8-(2,6-difluorophenyl)-4-(4-methylpiperazin-1-yl)-1,7-naphthyridine-3-carboxamide;
N-[(4S)-chroman-4-yl]-8-(3,5-dichlorophenyl)-4-pyrrolidin-1-yl-1,7-naphthyridine-3-carboxamide;
8-(3,5-dichlorophenyl)-4-(dimethylamino)-N-[(4S)-7-fluorochroman-4-yl]-1,7-naphthyridine-3-carboxamide;
N-[(4S)-chroman-4-yl]-4-(dimethylamino)-8-(1H-indol-4-yl)-1,7-naphthyridine-3-carboxamide;
N-[(4S)-chroman-4-yl]-8-(3,5-dichlorophenyl)-4-phenyl-1,7-naphthyridine-3-carboxamide;
N-[(4S)-chroman-4-yl]-4-cyclopropyl-8-(3,5-dichlorophenyl)-1,7-naphthyridine-3-carboxamide;
8-(3,5-dichlorophenyl)-4-(dimethylamino)-N-[(1S)-indan-1-yl]-1,7-naphthyridine-3-carboxamide;
N-[(4S)-6-cyanochroman-4-yl]-8-(3,5-dichlorophenyl)-4-(dimethylamino)-1,7-naphthyridine-3-carboxamide;
N-((4S)-7-chlorochroman-4-yl)-8-(3,5-dichlorophenyl)-4-(dimethylamino)-1,7-naphthyridine-3-carboxamide;
N-((4R)-7-chlorochroman-4-yl)-8-(3,5-dichlorophenyl)-4-(dimethylamino)-1,7-naphthyridine-3-carboxamide;
N-[(4S)-chroman-4-yl]-8-(2,6-difluoro-3-methoxy-phenyl)-4-(dimethylamino)-1,7-naphthyridine-3-carboxamide;
8-((4S)-3,5-dichlorophenyl)-4-(dimethylamino)-N-(7-methoxychroman-4-yl)-1,7-naphthyridine-3-carboxamide;
8-((4R)-3,5-dichlorophenyl)-4-(dimethylamino)-N-(7-methoxychroman-4-yl)-1,7-naphthyridine-3-carboxamide;
8-(3,5-dichlorophenyl)-4-(dimethylamino)-N-((8S)-5,6,7,8-tetrahydroquinolin-5-yl)-1,7-naphthyridine-3-carboxamide;
8-(3,5-dichlorophenyl)-4-(dimethylamino)-N-((8R)-5,6,7,8-tetrahydroquinolin-5-yl)-1,7-naphthyridine-3-carboxamide;
N-[(4S)-chroman-4-yl]-8-(3,5-dichloro-2-pyridyl)-4-(dimethylamino)-1,7-naphthyridine-3-carboxamide;
4-chloro-N-[(4S)-chroman-4-yl]-8-(3,5-dichlorophenyl)-1,7-naphthyridine-3-carboxamide;
N-[(4S)-chroman-4-yl]-8-(3,5-dichlorophenyl)-4-[methoxy(methyl)amino]-1,7-naphthyridine-3-carboxamide;
8-(3,5-dichlorophenyl)-4-(dimethylamino)-N-[(1S)-tetralin-1-yl]-1,7-naphthyridine-3-carboxamide;
N-[(4S)-chroman-4-yl]-8-(3,5-dichlorophenyl)-4-methoxy-1,7-naphthyridine-3-carboxamide;
8-(3,5-dichlorophenyl)-4-(dimethylamino)-N-[(1R)-indan-1-yl]-1,7-naphthyridine-3-carboxamide;
8-(3,5-dichlorophenyl)-4-(dimethylamino)-N-[(1R)-tetralin-1-yl]-1,7-naphthyridine-3-carboxamide;
8-(3,5-dichlorophenyl)-N-((8S)-6,7-dihydro-5H-cyclopenta[b]pyridin-5-yl)-4-(dimethylamino)-1,7-naphthyridine-3-carboxamide;
8-(3,5-dichlorophenyl)-N-((8R)-6,7-dihydro-5H-cyclopenta[b]pyridin-5-yl)-4-(dimethylamino)-1,7-naphthyridine-3-carboxamide;
N-[(4S)-chroman-4-yl]-8-(3,5-dichlorophenyl)-4-(4-oxoimidazolidin-1-yl)-1,7-naphthyridine-3-carboxamide;
8-(3,5-dichlorophenyl)-4-(dimethylamino)-N-[rac-(3R,4S)-3-methylchroman-4-yl]-1,7-naphthyridine-3-carboxamide;
N-[(4S)-chroman-4-yl]-4-cyano-8-(3,5-dichlorophenyl)-1,7-naphthyridine-3-carboxamide;
N-[(4S)-chroman-4-yl]-8-(3,5-dichlorophenyl)-4-isopropyl-1,7-naphthyridine-3-carboxamide;
N-[(4S)-chroman-4-yl]-4-(dimethylamino)-8-[3-(dimethylamino)-2,6-difluoro-phenyl]-1,7-naphthyridine-3-carboxamide;
8-(3,5-dichlorophenyl)-4-(dimethylamino)-N-[(4S)-7-methylchroman-4-yl]-1,7-naphthyridine-3-carboxamide;
8-(3,5-dichlorophenyl)-N-[(7S)-6,7-dihydro-5H-thieno[3,2-b]pyran-7-yl]-4-(dimethylamino)-1,7-naphthyridine-3-carboxamide;
8-(3,5-dichlorophenyl)-N-[(7R)-6,7-dihydro-5H-thieno[3,2-b]pyran-7-yl]-4-(dimethylamino)-1,7-naphthyridine-3-carboxamide;
N-[(4S)-chroman-4-yl]-8-(3,5-difluorophenyl)-4-(dimethylamino)-1,7-naphthyridine-3-carboxamide;
N-[(4S)-chroman-4-yl]-8-(2-fluorophenyl)-4-(dimethylamino)-1,7-naphthyridine-3-carboxamide;
N-[(4S)-chroman-4-yl]-4-(dimethylamino)-8-(2,3,5-trifluorophenyl)-1,7-naphthyridine-3-carboxamide;
N-[(4S)-chroman-4-yl]-4-(dimethylamino)-8-(3,4,5-trifluorophenyl)-1,7-naphthyridine-3-carboxamide;
8-[3,5-bis(trifluoromethyl)phenyl]-N-[(4S)-chroman-4-yl]-4-(dimethylamino)-1,7-naphthyridine-3-carboxamide;
8-(3,5-dichlorophenyl)-N-[(4S)-3,4-dihydro-2H-pyrano[3,2-c]pyridin-4-yl]-4-(dimethylamino)-1,7-naphthyridine-3-carboxamide;
N-[(4S)-chroman-4-yl]-8-(2,4-dichlorophenyl)-4-(dimethylamino)-1,7-naphthyridine-3-carboxamide;
N-[(4S)-chroman-4-yl]-8-(2,3-dichlorophenyl)-4-(dimethylamino)-1,7-naphthyridine-3-carboxamide;
N-[(4S)-chroman-4-yl]-8-(3,5-dichlorophenyl)-4-(dimethylamino) isoquinoline-3-carboxamide;
N-[(4S)-chroman-4-yl]-8-(3,5-dichlorophenyl)-4-(dimethylamino)-2,7-naphthyridine-3-carboxamide;
N-[(4S)-chroman-4-yl]-1-(3,5-dichlorophenyl)-5-(dimethylamino)isoquinoline-6-carboxamide;
(4S)-chroman-4-yl]-5-(3,5-dichlorophenyl)-1-(dimethylamino)naphthalene-2-carboxamide;
N-[(4S)-chroman-4-yl]-4-(3,5-dichlorophenyl)-8-(dimethylamino)quinoline-7-carboxamide; and
N-[(4S)-chroman-4-yl]-4-(3,5-dichlorophenyl)-8-(dimethylamino)isoquinoline-7-carboxamide; or a salt of each of the above named compounds.

14. A composition comprising a compound of claim 1, or a salt thereof, and at least one acceptable carrier.

15. A method for treating endoparasites in a subject, comprising administering to the subject a compound of claim 1, or a salt thereof.

16. A method for treating or controlling heartworm in a subject, comprising administering to the subject a compound of claim 1, or a salt thereof.

17. The compound according to claim 2, wherein n is 1; or a salt thereof.

18. The compound according to claim 3, wherein n is 1; or a salt thereof.

19. The compound according to claim 4, wherein n is 1; or a salt thereof.

20. The compound according to claim 5, wherein n is 1; or a salt thereof.
